(19) United States
(12) Patent Application Publication
(10) Pub. No.: US 2025/0002602 A1
(43) Pub. Date: Jan. 2, 2025

(54) IGA FC AND IGG FC TANDEM PROTEIN CONSTRUCTS

(71) Applicants: OSLO UNIVERSITETSSYKEHUS HF, Oslo (NO); UNIVERSITETET I OSLO, Oslo (NO)

(72) Inventors: Jan Terje ANDERSEN, Oslo (NO); Stian FOSS, Oslo (NO); Simone MESTER, Oslo (NO)

(21) Appl. No.: 18/707,069

(22) PCT Filed: Nov. 7, 2022
(86) PCT No.: PCT/EP2022/080998

§ 371 Date: May 2, 2024

(30) Foreign Application Priority Data
Nov. 5, 2021 (GB) 2115957.9

(51) Int. Cl. C07K 16/28; A61P 35/00; C07K 16/32

(52) U.S. Cl. CPC C07K 16/2887; A61P 35/00; C07K 16/32; C07K 2317/31; C07K 2317/522; C07K 2317/53; C07K 2317/72; C07K 2317/734

(57) ABSTRACT

The present invention provides a protein construct comprising a human IgA Fc region and a human IgG Fc region, wherein the C-terminals of the human IgA Fc region are connected to the N-terminals of the human IgG Fc region. Such constructs can further comprise a targeting domain. Compositions comprising said constructs and therapeutic uses of said constructs are also provided.

The present invention relates to generally to the field of protein constructs, in particular protein constructs comprising IgA Fc regions and IgG Fc regions connected in tandem. The invention also relates to such protein constructs further comprising a targeting domain or ligand, for example an antigen binding domain. The present invention also relates to compositions comprising such protein constructs, methods of producing such protein constructs, and therapeutic methods and uses which employ such protein constructs.

Monoclonal antibodies are used to treat an increasing range of diseases, where the approval rate in 2020 was the second highest ever, and dominated by antibodies targeting cancer.

For example, immunoglobulin (Ig) G-based therapeutics, for example IgG antibodies, may eliminate cancer cells via Fc mediated effector mechanisms, including by engaging the classical Fcγ receptors (FcγRs) for subsequent induction of effector functions, such as antibody-dependent cellular cytotoxicity (ADCC) and/or antibody-dependent cellular phagocytosis (ADCP). In addition, target binding of IgG antibodies may recruit the complement system followed by induction of complement-dependent cytotoxicity (CDC).

This has motivated development of Fc-engineering strategies that have given rise to IgG variants with enhanced effector functions. An additional advantage of using IgG antibodies is their long plasma half-life, which secures bioavailability via rescue from intracellular degradation by the neonatal Fc receptor (FcRn) that acts as a homeostatic regulator of IgG catabolismplasma half-life may further be extended by Fc-engineering for improved FcRn binding.

There is however a need for more potent formats, tailored for both optimal effector function properties and favorable pharmacokinetics.

The present invention provides an engineered antibody format that combines structural elements of IgA with that of IgG, allowing for efficient engagement of both FcγRs and FcαRs. Specifically, and surprisingly, an IgA Fc region connected to an IgG Fc region, wherein the C-terminals of the IgA Fc region are connected to the N-terminals of the IgG Fc region shows advantageous effector functions and efficient engagement of several effector molecules. In particular, the constructs of the present invention show improved binding to C1q, an important step in complement activation. In addition, the protein constructs of the present invention demonstrate a favorable plasma half-life by way of effective FcRn binding hence providing efficient rescue from intracellular degradation.

The present invention also surprisingly demonstrates that the protein constructs provided, in which the C-terminals of the IgA Fc region are connected to the N-terminals of the IgG Fc region, show improved properties over constructs engineered in the opposite orientation, i.e. in which the C-terminals of the IgG Fc region are connected to the N-terminals of the IgA Fc region.

Thus, in a first aspect, the present invention provides a protein construct comprising an IgA Fc region, preferably a human IgA Fc region, and an IgG Fc region, preferably a human IgG Fc region, wherein the C-terminals of the human IgA Fc region are connected to the N-terminals of the human IgG Fc region.

The term Fc region (or Fc fragment or fragment crystallizable region) as used herein comprises or corresponds to a part of an antibody that has the ability to interact with Fc receptors or which provides (or confers) an antibody effector function (e.g. antibody dependent cellular cytotoxicity). Naturally occurring Fc regions (or Fc fragments) are made up of two identical chains (are dimers) each chain containing (or comprising or consisting of) amino acid sequences of the CH2 and CH3 domains of an antibody. The two chains of the Fc fragment are generally connected to each other by at least one cysteine bridge (disulphide bond between cysteine residues).

Truncated, mutated or modified Fc regions (or Fc fragments), e.g. fragment or variant Fc regions, in particular fragments or variants of IgG-Fc regions or IgA-Fc regions, may be used and are included provided that the ability to interact with Fc receptors, e.g. the FcRn, and/or the ability to confer effector function is maintained or present, or improved, e.g. compared to the starting, non-mutated or wild-type Fc region. For example, appropriate mutants with improved binding to FcRs, e.g. FcRn, or appropriate mutants with (or which confer) improved or increased half-life or improved or increased effector function are well known and described in the art and any of these may be used.

The protein constructs of the invention thus comprise an Fc region (or Fc fragment) of an IgA antibody. Such an IgA Fc region (or fragment crystallizable region) has the ability to bind to FcαRI receptors and/or can provide (or confer) an appropriate IgA antibody effector function (e.g. antibody-dependent cellular cytotoxicity, ADCC, or antibody-dependent cellular phagocytosis, ADCP). As outlined above such Fc regions typically comprise CH2 and CH3 domains of an IgA antibody.

In some embodiments, the IgA Fc region used in the protein constructs of the present invention has a modified (or mutated or inactivated or truncated) tailpiece, or does not comprise a tailpiece (or the tailpiece has been removed).

Thus, IgA Fc regions (or Fc fragments) include those in which the tailpiece has been modified, mutated, inactivated, truncated or removed. Such IgA Fc regions still however comprise CH2 and CH3 domains of an IgA antibody. Preferred IgA Fc regions further comprise all or part of the IgA hinge region, e.g. at least the parts which are involved in the formation of di-sulphide bonds to link the two polypeptide chains comprising the IgA Fc region and/or the parts which are involved in Fc effector function.

A tailpiece is typically an 18 amino acid region (or extension) at the C-terminal end of an IgA heavy chain constant region or Fc region that contains a cysteine residue that is essential for polymerization (e.g. formation of dimers).

Thus, in preferred embodiments, in protein constructs of the present invention the IgA tailpiece has been modified, mutated, inactivated, truncated or removed. In some such embodiments, the cysteine residue that is essential for polymerization has been modified, mutated, inactivated, truncated or removed. Such removal or modification, etc., of the tailpiece is also convenient to allow connection of the C-terminals of the IgA Fc region to the N-terminals of the IgG Fc region.

The skilled person is familiar with IgA tailpieces (e.g. Janeway et al., 2001, Immunobiology: The Immune System in Health and Disease, 5^{th} Edition, New York: Garland Science). One example of an IgA1 tailpiece can be represented by the sequence PTHVNVSVVMAEVDGTCY (SEQ ID NO: 30).

Typically, there is at least one bond (preferably at least one disulphide bond) between the two polypeptide chains comprising the IgA Fc region. For example, there are typically disulphide bonds between the IgA antibody heavy chains.

The IgA Fc regions used in the constructs of the present invention can be derived from any subtype of IgA antibody, for example IgA1 or IgA2, preferably IgA2.

Thus, the present invention provides protein constructs wherein the IgA Fc region is an IgA1 Fc region or an IgA2 Fc region, preferably an IgA2 Fc region.

In some embodiments, the IgA Fc region may be engineered, or modified, to include enhanced or modified properties, e.g. enhanced or modified effector functions that may include the induction of antibody-dependent cellular cytotoxicity (ADCC) or antibody dependent cellular phagocytosis (ADCP), or increased co-engagement or binding to Fcα receptors, e.g. FcRαI. Appropriate Fc mutants with (or which confer) these features are well known and described in the art, and any of these may be used.

As mentioned above, appropriate Fc regions (IgA Fc regions) for use in the constructs of the present invention comprise the CH2 and CH3 domains. In some embodiments, CH1 domains can be included. However, in other embodiments CH2 and CH3 domains (or fragments or variants thereof which retain or have the ability to interact with FcαRs, e.g. FcαR1, or which provide or confer an IgA antibody effector function, and preferably the ability to dimerise, are the only parts of IgA antibodies included in the constructs. For example, in some embodiments no light chain antibody domains, e.g. no light chain antibody domains of an IgA antibody, in particular no light chain constant domains (CL domains) or light chain variable domains (VL) will be included in the constructs. In other examples, no heavy chain CH1 domains or heavy chain variable domains (VH), e.g. no heavy chain CH1 domains or heavy chain variable domains (VH) of an IgA antibody, will be included in the constructs. In other embodiments, as described elsewhere herein, a hinge region, e.g. an IgA hinge region or an IgG hinge region, will be included in the constructs. Where hinge regions are included, these can conveniently be included in their natural positions, i.e. linked or connected to the N-terminus of the IgA CH2 region, for example in between the IgA CH2 region and the CH1 region, where such CH1 regions are present. Alternatively, such hinge regions can be used to link the IgA CH2 domain of the constructs to a targeting domain as described elsewhere herein.

However, in some embodiments, for example embodiments where the IgA Fc region is part of an IgA antibody, light chain constant domains (CL domains) and/or light chain variable regions (VL), and/or heavy chain constant domains (CH1 domains) and/or heavy chain variable regions (VH) will be included. In other words, in some embodiments an antigen binding domain comprising VL and/or VH domains, optionally together with CL and/or CH1 domains will be present in the constructs. In preferred embodiments such antigen binding domains will be derived from IgA antibodies. In other preferred embodiments such antigen binding domains will be derived from IgG antibodies In preferred embodiments a hinge region, e.g. an IgA hinge region or an IgG hinge region, can be used to link the IgA CH2 region to the antigen binding domain, e.g. to the CH1 domain.

In preferred embodiments the IgA Fc regions are human IgA Fc regions. Sequences encoding constant regions of IgA antibodies and IgA Fc regions and the positions of the CH1, hinge, CH2 and CH3 domains are readily available to the skilled person. For example, the gene encoding human Immunoglobulin heavy constant alpha 1 is known as IGHA1 (see for example Uniprot: P01876) and the gene encoding human Immunoglobulin heavy constant alpha 2 is known as IGHA2 (see for example Uniprot: P01877).

For example, an exemplary IgA1 heavy chain constant region amino acid sequence is set forth herein as SEQ ID NO: 5, and an exemplary IgA2 heavy chain constant region amino acid sequence is set forth herein as SEQ ID NO: 6, from which exemplary sequences of CH1, CH2 and/or CH3 domains, as appropriate, preferably CH2 and CH3 domains, optionally together with the hinge region, can be derived for inclusion in the constructs. Exemplary sequences are provided in Table 1.

In some embodiments of the protein constructs of the present invention that comprise an IgA Fc region, the final (C-terminal-most) amino acid residue of the IgA Fc region is the residue corresponding to position 452 by Bur numbering (e.g. the A at position 452 by Bur numbering), see the various exemplary sequences in Table 1. The Bur numbering scheme is described by Liu et al. (Science. 1976 Sep. 10; 193 (4257): 1017-20) and is the preferred numbering system for the IgA molecules and fragments as described herein.

The protein constructs of the invention further comprise an Fc region (or Fc fragment) of an IgG antibody. Such an IgG Fc region (or fragment crystallizable region) has the ability to bind to IgG Fcγ receptors or FcRn or C1q, and/or can provide (or confer) an appropriate IgG antibody effector function (e.g. ADCC or ADCP, or complement-dependent cytotoxicity CDC). As outlined above such Fc regions typically comprise CH2 and CH3 domains of an IgG antibody.

Typically, there is at least one bond (preferably at least one disulphide bond) between the two polypeptide chains comprising the IgG Fc region. For example, there are typically disulphide bonds between the IgG antibody heavy chains. Preferred IgG Fc regions further comprise all or part of the IgG hinge region, e.g. at least the parts which are involved in the formation of di-sulphide bonds to link the two polypeptide chains comprising the IgG Fc region and/or the parts which are involved in Fc effector function.

The IgG Fc regions used in the constructs of the present invention can be derived from any subtype of IgG antibody, for example IgG1, IgG2, IgG3 or IgG4. In some embodiments, IgG1 or IgG2 Fc regions are used. In some embodiments preferably IgG1 Fc is used. In some embodiments preferably IgG2 Fc is used.

In one particular embodiment, a protein construct is provided, comprising a human IgA2 Fc region and a human IgG2 Fc region, wherein the C-terminals of the human IgA2 Fc region are connected to the N-terminals of the human IgG2 Fc region, and wherein the human IgG2 Fc region comprises:
(i) an arginine (R) residue at position 311 according to EU-numbering;
(ii) a glutamic acid (E) residue at position 428 according to EU-numbering; and
(iii) a tryptophan (W) residue at position 434 according to EU-numbering;
and wherein an IgG hinge is located between the IgA2 Fc region and the IgG2 Fc region.

In one particular embodiment, a protein construct is provided, comprising a human IgA2 Fc region and a human IgG2 Fc region, wherein the C-terminals of the human IgA2 Fc region are connected to the N-terminals of the human IgG2 Fc region, and wherein the human IgG2 Fc region comprises:
(i) an arginine (R) residue at position 311 according to EU-numbering;
(ii) a glutamic acid (E) residue at position 428 according to EU-numbering; and
(iii) a tryptophan (W) residue at position 434 according to EU-numbering;
and wherein a human IgG2 hinge is located between the IgA2 Fc region and the IgG2 Fc region.

In one particular embodiment, a protein construct is provided, comprising a human IgA2 Fc region and a human IgG2 Fc region, wherein the C-terminals of the human IgA2 Fc region are connected to the N-terminals of the human IgG2 Fc region, and wherein the human IgG2 Fc region comprises:
(i) an arginine (R) residue at position 311 according to EU-numbering;
(ii) an aspartic acid (D) residue at position 428 according to EU-numbering; and
(iii) a tryptophan (W) residue at position 434 according to EU-numbering; and wherein an IgG hinge is located between the IgA2 Fc region and the IgG2 Fc region.

In one particular embodiment, a protein construct is provided, comprising a human IgA2 Fc region and a human IgG2 Fc region, wherein the C-terminals of the human IgA2 Fc region are connected to the N-terminals of the human IgG2 Fc region, and wherein the human IgG2 Fc region comprises:
(i) a lysine (K) residue at position 311 according to EU-numbering;
(ii) an aspartic acid (D) residue at position 428 according to EU-numbering; and
(iii) a tryptophan (W) residue at position 434 according to EU-numbering; and wherein an IgG hinge is located between the IgA2 Fc region and the IgG2 Fc region.

In one particular embodiment, a protein construct is provided, comprising a human IgA2 Fc region and a human IgG3 Fc region, wherein the C-terminals of the human IgA2 Fc region are connected to the N-terminals of the human IgG3 Fc region, and wherein the human IgG3 Fc region comprises:
(i) an arginine (R) residue at position 311 according to EU-numbering;
(ii) a glutamic acid (E) residue at position 428 according to EU-numbering;
(iii) a tryptophan (W) residue at position 434 according to EU-numbering, and
(iv) a histidine (H) residue at position 435 according to EU-numbering; and and wherein an IgG hinge is located between the IgA2 Fc region and the IgG3 Fc region.

In all the above specific embodiments an IgG1 Fc region may be used in place of the IgG2 or IgG3 regions with the corresponding mutations.

Thus, the present invention provides protein constructs wherein the IgG Fc region is an IgG1 Fc region or an IgG2 Fc region, preferably an IgG1 Fc region.

In some embodiments, the IgG Fc region may be engineered, or modified, to include enhanced or modified properties, e.g. enhanced or modified effector functions that may include antibody-dependent cellular cytotoxicity (ADCC) or antibody-dependent cellular phagocytosis (ADCP), complement-dependent cytotoxicity (CDC), or an increased half-life or increased co-engagement or binding to Fcγ receptors.

As mentioned above, appropriate Fc regions (IgG Fc regions) for use in the constructs of the present invention comprise the CH2 and CH3 domains. Generally CH1 domains will not be included as part of the IgG Fc region. However, in some embodiments, a CH1 domain, e.g. an IgG CH1 domain, can be included in the constructs of the invention, for example positioned N-terminally of the CH2 and CH3 domains of the IgG Fc region, for example positioned between the IgA Fc region and the IgG Fc region of the constructs. However, in other embodiments, CH2 and CH3 domains (or fragments or variants thereof which retain or have the ability to interact with FcgRs or FcRn or C1q, or which provide or confer an IgG antibody effector function, and preferably the ability to dimerise) are the only parts of IgG antibodies included in the constructs. In other embodiments, as described elsewhere herein, a hinge region, e.g. an IgG hinge region, will be included in the constructs. Where hinge regions are included these can conveniently be included in their natural positions, i.e. linked or connected to the N-terminus of the IgG CH2 region, for example in between the IgG CH2 region and the IgA CH3 region in the tandem constructs of the invention. Thus, preferably, such hinge regions can be used to link the IgG CH2 domain of the constructs to the IgA CH3 domain of the constructs. In other words can be used to link the IgG Fc region to the IgA Fc region.

However, in some embodiments, for example embodiments where the IgA Fc region is connected to an antigen binding domain, light chain constant domains (CL domains) and/or light chain variable regions (VL), and/or heavy chain constant domains (CH1 domains) and/or heavy chain variable regions (VH) can be included. In other words, in some embodiments an antigen binding domain comprising VL and/or VH domains, optionally together with CL and/or CH1 domains will be present in the constructs. In some embodiments such antigen binding domains can be derived from IgG antibodies.

In preferred embodiments the IgG Fc regions are human IgG Fc regions. Sequences encoding constant regions of IgG antibodies and IgG Fc regions and the positions of the CH1, hinge, CH2 and CH3 domains are readily available to the skilled person. For example, the gene encoding human Immunoglobulin heavy constant gamma 1 is known as IGHG1 (see for example Uniprot: P01857), the gene encoding human Immunoglobulin heavy constant gamma 2 is known as IGHG2 (see for example Uniprot: P01859), the gene encoding human Immunoglobulin heavy constant gamma 3 is known as IGHG3 (see for example Uniprot: P01860) and the gene encoding human Immunoglobulin heavy constant gamma 4 is known as IGHG4 (see for example Uniprot: P01861).

For example, an exemplary IgG1 heavy chain constant region amino acid sequence is set forth herein as SEQ ID NO: 1, an exemplary IgG2 heavy chain constant region amino acid sequence is set forth herein as SEQ ID NO: 2, an exemplary IgG3 heavy chain constant region amino acid sequence is set forth herein as SEQ ID NO: 3 and an exemplary IgG4 heavy chain constant region amino acid sequence is set forth herein as SEQ ID NO: 4, from which exemplary sequences of CH1, CH2 and/or CH3 domains, as appropriate, preferably CH2 and CH3 domains, optionally together with the hinge region, can be derived for inclusion in the constructs. Exemplary sequences are shown in Table 1. Engineered IgG heavy chain constant regions and IgG Fc variants with modified glycosylation pattern and/or stability are also well known. The well-known EU numbering scheme is the preferred numbering system for the IgG molecules and fragments as described herein.

In the constructs of the present invention the orientation of the Fc regions is important. Thus, the IgA Fc region is positioned to the N-terminus of the IgG Fc region. Specifically, the C-terminals of the IgA Fc region are connected to the N-terminals of the IgG Fc region, i.e. the C-terminus or the C-terminal end of each of the two polypeptides making up the IgA Fc region are connected to the N-terminus or the N-terminal end of each of the two polypeptides making up the IgG Fc region. The C-terminals of the IgA Fc region can be connected to the N-terminals of the IgG Fc region directly, i.e. without a linker. However, the C-terminals of the IgA Fc region can be connected to the N-terminals of the IgG Fc region indirectly, i.e. with a linker as described below.

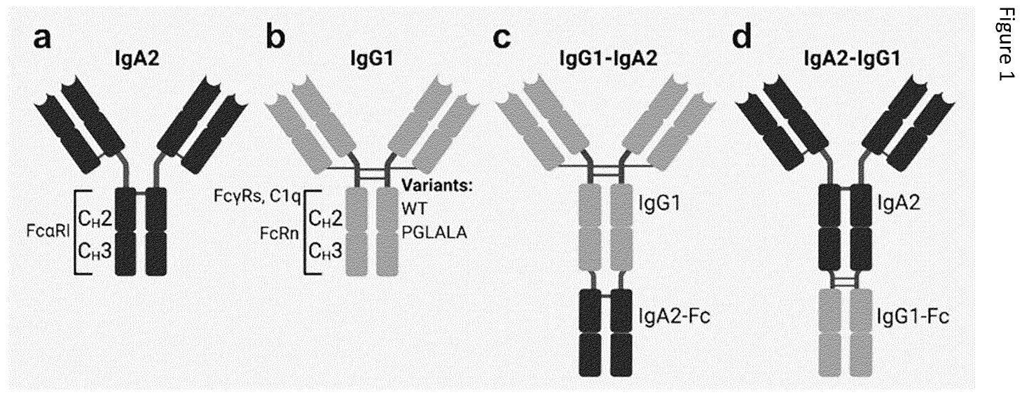

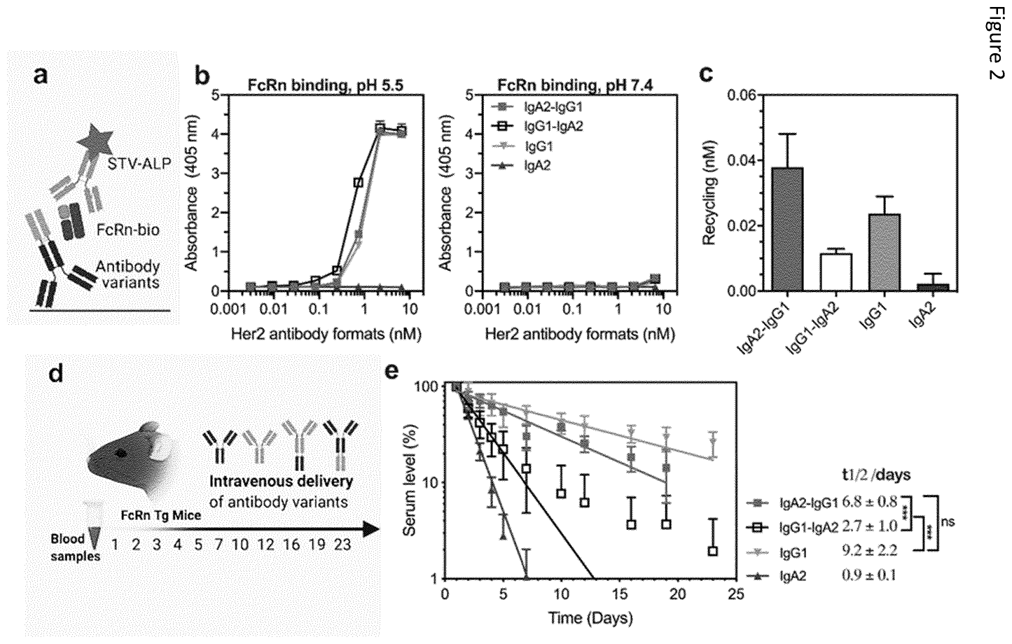

In the constructs of the present invention, both the IgA Fc region and IgG Fc region are positioned such that functionality of each Fc region is at least retained, and preferably improved. Appropriate molecules and constructs for comparison would be well known to a person skilled in the art. For example, retention or improvements can be judged when compared to an IgA Fc region or IgG Fc region alone, e.g. in an appropriate full-length IgG or IgA antibody (see for example FIG. 1a or 1b), or when compared to an equivalent protein construct to the protein construct of the invention but wherein the orientation of the IgA Fc region and IgG Fc region are reversed, i.e. in constructs wherein the C-terminals of the IgG Fc region are connected to the N-terminals of the IgA Fc region. An exemplary such molecule is shown in FIG. 1c.

Thus, constructs of the present invention are capable of binding to appropriate FcRs, e.g. FcαRs or FcγRs, or FcRn, and/or are capable of Fc effector function, for example one or more of the Fc effector functions selected from: the ability to induce ADCC, the ability to induce CDC, and the ability to induce ADCP. As is well known and described in the art, the ability to induce ADCC and the ability to induce ADCP takes place via binding of an appropriate Fc region to an appropriate FcR on various cell types that are capable of such functions, e.g. macrophages in the case of ADCP, PMNs or NK cells for ADCC. The ability to induce CDC generally takes place via binding of an Fc region to a component of the C1 complex, preferably C1q. The ability to bind FcRn is important for plasma half-life.

Thus, constructs of the present invention are capable of binding to FcRs or are capable of Fc effector function. For example, said constructs can bind FcαRs, e.g. Fcα1, FcγRs, e.g. one or more of FcγRI, FcγRIIa, FcγRIIb, FcγRIIc, FcγRIIIa, or FcγRIIIb, and/or FcRn.

Alternatively, or in addition, said constructs can induce CDC, induce ADCC and/or induce ADCP. In order to induce CDC, preferred constructs of the invention can bind C1q.

Preferably, protein constructs in accordance with the present invention, which comprise an IgA Fc region (or Fc fragment), have or retain the ability to bind to FcαR (e.g. human FcαR). FcαR may also be referred to as FcαRI or CD89. In some embodiments, protein constructs of the invention have the ability to bind to a recombinant version of FcαRI (e.g. recombinant human FcαRI). An IgA Fc region in the protein constructs of the PI binding to (or cross-linking with) FcαRI expressed for example on polymorphonuclear leukocytes (PMNs) may result in antibody-dependent cytotoxicity (ADCC). Thus, it is preferable that the inclusion of both an IgA Fc region and an IgG Fc region in the constructs of the present invention does not significantly affect the ability of said construct to bind to FcαRI. The inventors have reported herein (see Example 1) that the inclusion of both an IgA Fc region and an IgG Fc region in the constructs of the present invention does not significantly affect or reduce the ability of said constructs to bind to FcαRI. In other words, the constructs of the present invention can bind (or still bind) to FcαRI.

Thus, in some embodiments, a protein construct of the present invention comprising an IgA Fc region has an ability to bind to FcαRI that is not significantly altered (or not altered) or is substantially equivalent to (or equivalent to or comparable to), or is improved over, the ability of a control protein to bind to FcαRI, for example a control protein that comprises an IgA Fc region, e.g. an IgA antibody, e.g. a full-length IgA antibody (e.g. a wild-type full-length IgA antibody) of the appropriate sub-type such as an IgA1 or IgA2 antibody (see FIG. 1a), selected for example depending on the subtype of the IgA Fc region included in the construct of the invention, or another appropriate construct that contains a single IgA Fc region, e.g. contains a single IgA Fc region which is the only Fc region in the construct. Preferably, a control protein is substantially identical to (or identical to) or equivalent to the protein construct of the invention with which the comparison is being made, with the exception (or difference) being that the control protein construct does not comprise an IgG Fc region in accordance with the invention. In some embodiments, the protein construct of the invention comprises an IgA antibody and an exemplary control protein comprises the same IgA antibody with the sole difference (as compared to the protein of the invention) being that the control protein does not comprise an IgG Fc region in accordance with the invention.

Appropriate control proteins will preferably contain the same or equivalent targeting domain or antigen binding domain as the protein construct of the invention.

A level of binding to FcαRI so as to be functionally effective, e.g. at a level to allow Fc effector function, e.g. ADCC, is desired. The ability of a protein construct of the invention to bind to FcαRI may be assessed by any appropriate method or assay and the skilled person is familiar with appropriate assays. The discussion above may be in relation to FcαRI binding as determined by (or as assessed by) any appropriate assay. Typically, and preferably, an ELISA assay is used. A particularly preferred assay for determining FcαRI binding of protein constructs of the invention is described in Example 1 herein.

Preferably, protein constructs in accordance with the present invention, which comprise an IgG Fc region (or Fc fragment), have or retain the ability to bind to FcγRs (e.g. human FcγRs). There are three classes of receptors for human IgG found on leukocytes; FcγRI (CD64), FcγRIIa, FcγRIIb, FcγRIIc (CD32), and FcγRIIIa, FcγRIIIb (CD16), and the protein constructs of the invention have the ability to bind to one or more, or preferably all of these Fcγ receptors. In some embodiments, protein constructs of the invention have the ability to bind to a recombinant version of FcγRs (e.g. recombinant human FcγRs). An IgG Fc region in the protein constructs of the present invention binding to (or cross-linking with) FcγRs expressed for example on effector cells such as natural killer cells, macrophages, monocytes or eosinophils, may result in antibody-dependent cytotoxicity (ADCC). Thus, it is preferable that the inclusion of both an IgA Fc region and an IgG Fc region in the constructs of the present invention does not significantly affect the ability of said construct to bind to FcγRs.

The inventors have reported herein (see Example 1 and Example 2) that the inclusion of both an IgA Fc region and an IgG Fc region in the constructs of the present invention does not significantly reduce the ability of said constructs to bind to FcγRs, e.g. compared to a full-length IgG antibody of the appropriate subtype (selected for example depending on the subtype of the IgG Fc region included in the construct of the invention). Binding to each of the three classes of FcγRs is tested and the constructs of the present invention show good binding to one or more of these. Binding to FcγRI, FcγRIIa (both R131 and H131 variants), FcγRIIb, FcγRIIIa (both F158 and V158 variants), and FcγRIIIb have been specifically tested. In all cases the binding to FcγRs is at least equivalent to the binding of an appropriate full-length IgG antibody. Surprisingly, in some cases, specifically the binding to FcγRI, FcγRIIa (both R131 and H131 variants), FcγRIIb, FcγRIIIa-V, and FcγRIIIb, the binding of the construct of the present invention is improved (increased) or superior to the binding of the full-length IgG antibody. Also in some cases, the binding of the construct of the present invention is improved (increased) or superior to the binding of a construct with reverse orientation to that of the present invention, i.e. a construct in which the C-terminals of the IgG Fc region are connected to the N-terminals of the IgA Fc region.

NK cells express FcγRIIIa and are thought to be the main cell population to mediate ADCC upon cross-linking of IgG antibodies. Thus, the improved ability of constructs of the invention to bind to FcγRIIIa is believed to be particularly advantageous in terms of enabling an improved ability to bind to NK cells and thereby efficiently mediate ADCC.

Thus, in some embodiments, a protein construct of the present invention comprising an IgG Fc region has an ability to bind to FcγRs that is not significantly altered (or not altered) or is substantially equivalent to (or equivalent to or comparable to), or is improved (or increased) over, the ability of a control protein to bind to the equivalent FcγR, for example a control protein that comprises an IgG Fc region, e.g. an IgG antibody, e.g. a full-length IgG antibody (e.g. a wild-type full-length IgG antibody) of the appropriate sub-type such as an IgG1, IgG2, IgG3 or IgG4 antibody, or another appropriate construct that contains a single IgG Fc region, e.g. contains a single IgG Fc region which is the only Fc region in the construct. Preferably, a control protein is substantially identical to (or identical to) or equivalent to the protein construct of the invention with which the comparison is being made, with the exception (or difference) being that the control protein construct does not comprise an IgA Fc region in accordance with the invention.

Appropriate control proteins will preferably contain the same or equivalent targeting domain or antigen binding domain as the protein construct of the invention.

A level of binding to FcγRs, e.g. any of the FcγRs mentioned above, so as to be functionally effective, e.g. at a level to allow Fc effector function, e.g. ADCC, is desired. The ability of a protein construct of the invention to bind to FcγRs may be assessed by any appropriate method or assay and the skilled person is familiar with appropriate assays. The discussion above may be in relation to FcγR binding as determined by (or as assessed by) any appropriate assay. Typically, and preferably, an ELISA assay is used. A particularly preferred assay for determining FcγR binding of protein constructs of the invention is described in Example 1 herein.

FcRn is a type 1 membrane glycoprotein which is largely expressed within acidic intracellular compartments such as endosomes. One of the known roles for FcRn is in recycling of certain molecules such as IgG or albumin back to the serum following endocytosis. For example, FcRn interacts with the Fc region of IgG at the CH2-CH3 domain interface with 2:1 stoichiometry (i.e. one molecule of IgG-Fc binds to two molecules of FcRn). Recycling is facilitated by pH-dependent binding of IgG-Fc to FcRn. In this regard, IgG-Fc binds FcRn with high affinity at pH 6.0/6.5, but not at pH 7.4. In this way, FcRn binds to IgG in the acidified endosomes (via the IgG-Fc region), but IgG then dissociates from FcRn at physiological/neutral pH, e.g. when the recycling endosomes containing FcRn-IgG complexes fuse with the cell membrane thereby releasing IgG back into the serum. By this mechanism IgG may avoid lysosomal degradation and FcRn mediated recycling is believed to be responsible for the long half-life of IgG in the circulation (a serum half life of around 21 days for IgG1, IgG2 and IgG4 and a serum half-life of around 7 days for IgG3 which has less efficient binding to FcRn). Fc regions of IgA antibodies do not show good ability to bind to FcRn which is believed to contribute to their relatively poor serum half-life (of around 1 day).

Preferably, protein constructs in accordance with the present invention, which comprise an IgG Fc region (or Fc fragment), have or retain the ability to bind to FcRn (e.g. human FcRn), preferably in a pH dependent manner. In some embodiments, protein constructs of the invention have the ability to bind to a recombinant version of FcRn (e.g. recombinant human FcRn). As discussed elsewhere herein, IgG Fc regions bind FcRn meaning that the IgG antibody is rescued from degradation by a pH dependent mechanism and have a relatively long serum half-life. Thus, it is preferable that the inclusion of both an IgA Fc region and an IgG Fc region in the constructs of the present invention does not significantly affect the ability of said construct to bind to FcRn in a pH dependent manner. The inventors have reported herein (see Example 1) that the inclusion of both an IgA Fc region and an IgG Fc region in the constructs of the present invention does not significantly affect or reduce the ability of said constructs to bind to FcRn in a pH dependent manner, e.g. compared to a full-length IgG antibody of the appropriate subtype (selected for example depending on the subtype of the IgG Fc region included in the construct of the invention).

Thus, in some embodiments, a protein construct of the present invention has the ability to bind to FcRn, preferably in a pH-dependent manner. More specifically, preferably a protein construct of the present invention has an ability to bind to FcRn at acidic pH (e.g. pH 5.5) but has no significant ability (or no ability) to bind to (or shows no or no significant binding to or significantly reduced binding to or a weak or low affinity binding to) FcRn at neutral pH (e.g. pH 7.4). Surprisingly, the inventors have shown (see Example 1) that the ability of constructs of the present invention to bind to FcRn is improved (or increased) or superior as compared to the binding of a full-length IgG antibody. Also, the binding of the constructs of the present invention to FcRn have been shown to be improved (or increased) or superior to the binding of a construct with reverse orientation to that of the present invention, i.e. a construct in which the C-terminals of the IgG Fc region are connected to the N-terminals of the IgA Fc region. In some embodiments, the inclusion of REW mutations as described elsewhere herein can improve the FcRn binding even further.

Thus, this ability for constructs of the invention to bind to FcRn, preferably in a pH-dependent manner, is not significantly altered (or not altered) or is substantially equivalent to (or equivalent to or comparable to), or is improved (increased) over, the ability of a control protein to bind to FcRn, for example a control protein that comprises an IgG Fc region, e.g. an IgG antibody, e.g. a full-length IgG antibody (e.g. a wild-type full-length IgG antibody) of the appropriate sub-type such as an IgG1, IgG2, IgG3 or IgG4 antibody, or another appropriate construct that contains a single IgG Fc region, e.g. contains a single IgG Fc region which is the only Fc region in the construct. Preferably, a control protein is substantially identical to (or identical to) or equivalent to the protein construct of the invention with which the comparison is being made, with the exception (or difference) being that the control protein construct does not comprise an IgA Fc region in accordance with the invention. Appropriate control proteins will preferably contain the same or equivalent targeting domain or antigen binding domain as the protein construct of the invention.

A level of binding to FcRn so as to be functionally effective, e.g. at a level to allow recycling of the construct and/or an equivalent or improved serum half-life is desired. The ability of a protein construct of the invention to bind to FcRn may be assessed by any appropriate method or assay and the skilled person is familiar with appropriate assays. The discussion above may be in relation to FcRn binding as determined by (or as assessed by) any appropriate assay. Typically, and preferably, an ELISA assay is used. Typically, and preferably, parallel ELISA assays may be performed, one at neutral pH (e.g. pH 7.4) and one at an acidic pH (e.g. pH 5.5) in order to assess the pH-dependency of FcRn binding. A particularly preferred assay for determining FcRn binding of protein constructs of the invention is described in Example 1 herein.

Preferably, protein constructs in accordance with the present invention may be recycled and rescued from intracellular degradation (e.g. from lysosomal degradation) in an FcRn-dependent (or FcRn-mediated) manner. As discussed elsewhere herein, IgA antibodies typically suffer from having a short in vivo half-life (e.g. a half-life of around 1 day). The inventors have demonstrated herein (see Example 1) that the inclusion of both an IgA Fc region and an IgG Fc region in the constructs of the present invention can confer upon IgA antibodies (or on other constructs where an IgA Fc region is present or is the only Fc region) the ability (or enhance the ability or increase the ability) to be recycled and rescued via an FcRn-dependent mechanism.

In addition, the inventors have demonstrated herein (see Example 1) that the inclusion of both an IgA Fc region and an IgG Fc region in the constructs of the present invention does not reduce the ability of said constructs, which comprise an IgG Fc region (or Fc fragment), to be recycled and rescued via an FcRn-dependent mechanism. Indeed, the inventors have shown that the recycling and rescue via FcRn for the constructs of the invention is at least equivalent to the recycling and rescue of an appropriate full-length IgG antibody, e.g. an IgG1 antibody. Surprisingly, the recycling and rescue via FcRn of a construct of the present invention is shown to be improved (increased) or superior to the recycling and rescue of a full-length IgG antibody. Also, the recycling and rescue via FcRn of a construct of the present invention is shown to be improved (increased) or superior to the recycling and rescue of a construct with reverse orientation to that of the present invention, i.e. a construct in which the C-terminals of the IgG Fc region are connected to the N-terminals of the IgA Fc region. Without wishing to be bound by theory, it is believed that this ability to bind to FcRn and to be recycled and rescued is responsible for the increased in vivo half-life observed for protein constructs in accordance with the invention (as discussed elsewhere herein).

In preferred embodiments, the ability of a protein construct of the invention to be recycled and rescued via an FcRn-dependent mechanism is maintained or not significantly altered (e.g. as compared to an IgG Fc containing control protein), or increased (preferably significantly increased) as compared to the ability of a control protein to be recycled and rescued via an FcRn-dependent mechanism.

In some embodiments, the ability of a protein construct of the invention to be recycled and rescued via an FcRn-dependent mechanism is increased, for example, by at least 1.2-fold, at least 1.3-fold, at least 1.4-fold, at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold, as compared to the ability of a control protein to be recycled and rescued via a FcRn-dependent mechanism (e.g. there is an up to 1.5-fold, up to 2-fold, up to 3-fold, up to 4-fold, up to 5-fold, up to 6-fold, up to 7-fold, up to 8-fold, up to 9-fold, up to 10-fold, up to 11-fold, up to 12-fold, up to 13-fold, up to 14-fold, up to 15-fold, up to 20-fold, up to 50-fold or up to 100-fold increase, such as a 1.2 or 1.5-fold to 100-fold increase, 1.2 or 1.5-fold to 50-fold, 1.2 or 1.5-fold to 20-fold increase, 1.2 or 1.5-fold to 10-fold increase, 3-fold to 100-fold increase, 3-fold to 50-fold increase, 3-fold to 20-fold increase, 3-fold to 10-fold increase, 5-fold to 100-fold increase, 5-fold to 50-fold, 5-fold to 20-fold or a 5-fold to 10-fold increase, 10-fold to 100-fold increase, 10-fold to 50-fold, 10-fold to 20-fold or a 10-fold to 15-fold increase).

Where an increase in recycling and rescue is achieved an appropriate control protein comprises an IgA Fc region, e.g. is an IgA antibody, e.g. a full-length IgA antibody (e.g. a wild-type full-length IgA antibody) of the appropriate sub-type such as an IgA1 or IgA2 antibody selected for example depending on the subtype of the IgA Fc region included in the construct of the invention, or another appropriate construct that contains a single IgA Fc region, e.g. contains a single IgA Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgG Fc region in accordance with the invention. In some embodiments, the protein construct of the invention comprises an IgA antibody and an exemplary control protein comprises the same IgA antibody with the sole difference (as compared to the protein of the invention) being that the control protein does not comprise an IgG Fc region in accordance with the invention.

Alternatively, where an increase in recycling and rescue is achieved, or wherein a maintenance of recycling and rescue is observed, e.g, wherein the recycling and rescue is not significantly altered (or not altered) or is substantially equivalent to (or equivalent to or comparable to), the recycling and rescue ability of a control protein, an appropriate control protein may comprise an IgG Fc region, e.g. an IgG antibody, e.g. a full-length IgG antibody (e.g. a wild-type full-length IgG antibody) of the appropriate sub-type such as an IgG1, IgG2, IgG3 or IgG4 antibody, or another appropriate construct that contains a single IgG Fc region, e.g. contains a single IgG Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgA Fc region in accordance with the invention.

Appropriate control proteins will preferably contain the same or equivalent targeting domain or antigen binding domain as the protein construct of the invention.

The ability of a protein construct of the invention to be recycled and rescued via an FcRn-dependent mechanism may be as determined by any suitable assay or method and a skilled person will be familiar with suitable assays or methods. The discussion above may be in relation to recycling and rescuing via an FcRn-dependent mechanism as determined by (or as assessed by) any appropriate assay.

In some embodiments, the ability of a protein of the invention to be recycled and rescued via an FcRn-dependent mechanism is as determined in (or as assessed in) a human endothelial cell-based recycling assay (HERA). Such HERAs can also be used to assess the ability of protein constructs of the invention to bind to FcRn in a pH-dependent manner. Suitable HERAs are known in the art (e.g. Grevys et al., 2018, Nat. Commun., 9 (1): 621). An exemplary HERA for determining the ability of a protein construct of the invention to be recycled and rescued via an FcRn-dependent mechanism is described in Example 1 herein.

Typically, and preferably, protein constructs in accordance with the present invention have a therapeutically useful in vivo half-life (e.g. a therapeutically useful half-life in the mammalian, preferably human, circulation, or a therapeutically useful serum half-life or a therapeutically useful plasma half-life). IgA antibodies typically suffer from having a short in vivo half-life (e.g. a half-life of around 1 day). The inventors have demonstrated herein (see Example 1) that the inclusion of both an IgA Fc region and an IgG Fc region in the constructs of the present invention does not significantly reduce the in vivo half-life of said construct. Indeed, the constructs of the present invention show significant extension (or increase) of in vivo half-life of IgA based proteins, e.g. IgA antibodies (e.g. full-length, e.g. wild-type, IgA1 or IgA2 antibodies) or IgA based antibodies (e.g. IgA1 or IgA2 based antibodies).

This is an advantageous property which means that IgA antibodies or protein constructs which comprise an IgA Fc region, and in particular comprise an IgA Fc region as the only Fc region, which otherwise may be of no (or sub-optimal) in vivo (e.g. therapeutic) use due to their short half-life can be modified by preparing or incorporating them into a protein construct in accordance with the present invention to extend their half-life.

In addition, the inventors have demonstrated herein (see Example 1) that the inclusion of both an IgA Fc region and an IgG Fc region in the constructs of the present invention does not significantly reduce the in vivo half-life of said constructs. Indeed, the inventors have shown that the in vivo half-life of the constructs of the invention is not significantly different to or equivalent to (or only slightly shorter than) the in vivo half-life of an appropriate full-length IgG antibody, e.g. an IgG1 antibody. Also, the in vivo half-life of a construct of the present invention is shown to be significantly improved (increased) or superior to the in vivo half-life of a construct with reverse orientation to that of the present invention, i.e. a construct in which the C-terminals of the IgG Fc region are connected to the N-terminals of the IgA Fc region. In some embodiments, the inclusion of REW mutations as described elsewhere herein can improve the in vivo half-life even further.

In preferred embodiments, the in vivo half-life of a protein construct of the invention is maintained or not significantly altered (e.g. as compared to an IgG Fc containing control protein), or increased (preferably significantly increased) as compared to the in vivo half-life of a control protein.

In some embodiments, a protein construct of the present invention has an in vivo half-life of at least 3 days, or at least 4 days, or at least 5 days or at least 6 days or at least 7 days (e.g. up to 4 days, up to 5 days, up to 6 days, up to 7 days, up to 8 days, up to 10 days, up to 15 days, up to 20 days, up to 22 days, or up to 25 days, for example 3-5 days, 3-7 days, 4-7 days or 5-7 days or 6-7 days or 4-22 days or 5-22 days or 6-22 days). In preferred embodiments, the in vivo half-life is the in vivo half-life in a mammal, e.g. as assessed in an experimental animal such as a mouse, or in a human. The in vivo half-life may be the serum half-life or plasma half-life (or half-life in the circulation or in the bloodstream) in an experimental animal such as a mouse, in particular mice which express human FcRn, but not mouse FcRn, or in a human. In some embodiments, the in vivo half-life is the β-phase half-life.

In some preferred embodiments, protein constructs of the present invention, for example proteins comprising IgA antibodies (e.g. IgA1 or IgA2 antibodies), have an in vivo half-life that is increased (preferably significantly increased) in comparison to the in vivo half-life of a control protein.

In some embodiments, the in vivo half-life of a protein construct in accordance with the present invention (e.g. proteins comprising IgA antibodies (e.g. IgA1 or IgA2 antibodies), is increased by at least 3-fold, at least 4-fold, at least 5-fold, at least 6-fold, at least 7-fold, at least 8-fold, at least 9-fold or at least 10-fold as compared to the in vivo half-life of a control protein (e.g. there is an up to 3-fold, up to 4-fold, up to 5-fold, up to 6-fold, up to 7-fold, up to 8-fold, up to 9-fold, up to 10-fold, up to 15-fold, or up to 20-fold increase, such as a 3-fold to 5-fold increase, a 3-fold to 8-fold increase, a 3-fold to 10-fold increase, a 3-fold to 20-fold increase, a 5-fold to 8-fold increase, a 5-fold to 10-fold increase or a 5-fold to 20-fold increase), for example as compared to the in vivo half-life of a control protein.

Where an increase in in vivo half-life is achieved an appropriate control protein comprises an IgA Fc region, e.g. is an IgA antibody, e.g. a full-length IgA antibody (e.g. a wild-type full-length IgA antibody) of the appropriate sub-type such as an IgA1 or IgA2 antibody selected for example depending on the subtype of the IgA Fc region included in the construct of the invention, or another appropriate construct that contains a single IgA Fc region, e.g. contains a single IgA Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgG Fc region in accordance with the invention. In some embodiments, the protein construct of the invention comprises an IgA antibody and an exemplary control protein comprises the same IgA antibody with the sole difference (as compared to the protein of the invention) being that the control protein does not comprise an IgG Fc region in accordance with the invention.

Alternatively, where a maintenance or at least a maintenance of in vivo half-life is observed, e.g, wherein the in vivo half-life is not significantly altered (or not altered) or is substantially equivalent to (or equivalent to or comparable to), the in vivo half-life of a control protein, an appropriate control protein may comprise an IgG Fc region, e.g. an IgG antibody, e.g. a full-length IgG antibody (e.g. a wild-type full-length IgG antibody) of the appropriate sub-type such as an IgG1, IgG2, IgG3 or IgG4 antibody, or another appropriate construct that contains a single IgG Fc region, e.g. contains a single IgG Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgA Fc region in accordance with the invention.

Appropriate control proteins will preferably contain the same or equivalent targeting domain or antigen binding domain as the protein construct of the invention.

In vivo half-life (or plasma half-life or serum half-life) may be as determined by any suitable method and a skilled person will be familiar with suitable methods. In some embodiments, in vivo half-life is as determined in an experimental animal (e.g. an experimental mouse) and in some embodiments the discussion and values above in relation to in vivo half-life relate to in vivo half-life as determined in a mouse model (e.g. as described elsewhere herein). In other embodiments the discussion and values above in relation to in vivo half-life relate to in vivo half-life as observed in humans.

In a preferred such method for determining in vivo half-life, mice are used to determine the half-life of protein constructs in accordance with the invention, e.g. mice which express human FcRn, but not mouse FcRn (such as homozygote Tg32 mice (B6.Cg-Fcgrttm1Dcr Tg (FCGRT)32Dcr/DcrJ; The Jackson Laboratory)). Preferred mice are described in the Example 1. An exemplary method for determining in vivo half-life is described in Example 1 herein.

Advantageously, the inventors have demonstrated herein (see Example 1), that constructs of the present invention also perform well in a mouse model which has been adapted to more closely represent a true in vivo situation or a physiologically relevant situation. In this mouse model, e.g. a modification of the mouse model described above, the mice are pre-loaded with a pooled collection of human IgG molecules, e.g. in the form of an IVIg preparation. The presence of an excess of such human IgG molecules is thought to more accurately represent the situation in vivo, where such molecules are present for example in the form of endogenous antibodies, and such high levels of IgG will compete for binding and recycling of FcRn. Competition for the IgG binding site on FcRn may thus be present. Advantageously, the inventors have demonstrated that the constructs of the invention still show a favourable half-life in the presence of said competing IgG molecules. Thus, in such models the in vivo half-life of the constructs of the invention is not significantly altered (or not altered) or substantially equivalent to (or equivalent to or comparable to), or only slightly shorter than, the in vivo half-life of an appropriate control protein as described elsewhere herein, for example a wild-type full-length IgG antibody such as IgG1. This effect is demonstrated in human FcRn transgenic mice e.g. as described above and in Example 1. In some embodiments, the inclusion of REW mutations as described elsewhere herein can improve the in vivo half-life further in such models.

Another important effector function that some antibodies have is the ability to activate the classical complement pathway (known as complement dependent cytotoxicity or CDC). The ability of an antibody or other appropriate protein construct to induce CDC is generally mediated by binding of an Fc region of the antibody or construct to a component of the C1 complex, preferably C1q. This interaction can then induce lysis of the target cell to which the antibodies (or constructs) are bound, through CDC. In general, the ability to induce CDC varies with the Fc region concerned and whether or not (or how well) the Fc region can bind C1q. For example, wild-type human IgG1 Fc and IgG3 Fc regions generally have good ability to bind to C1q and induce CDC. IgG2 Fc regions or IgG4 Fc regions generally only have weak or low ability to do this. Human IgA Fc regions do not generally have the ability or have only a weak ability to bind to C1q and induce CDC.

Preferably, protein constructs in accordance with the present invention have or retain the ability to bind to C1q (e.g. human C1q), or show an increased or improved ability to bind C1q (e.g. human C1q).

Thus, in turn, protein constructs in accordance with the present invention preferably have or retain the ability to induce CDC, or show an increased or improved ability to induce CDC.

In some embodiments, protein constructs of the invention have the ability to bind to a recombinant version of C1q (e.g. recombinant human C1q). As discussed elsewhere herein, IgG Fc regions, in particular IgG1 and IgG3 Fc regions bind well to C1q meaning that antibodies of these serotypes generally show good CDC activity. Thus, in some embodiments, protein constructs containing an IgG1 Fc region or an IgG3 Fc region, in particular an IgG1 Fc region are preferred. However, IgA Fc regions, e.g. IgA1 and IgA2 Fc regions generally have poor or no CDC activity. Thus, it is preferable that the inclusion of both an IgA Fc region and an IgG Fc region in the constructs of the present invention does not significantly affect the ability of said construct to bind to C1q, e.g. via the IgG Fc region. In addition, it is preferable that the inclusion of both an IgA Fc region and an IgG Fc region in the constructs of the present invention can confer upon IgA antibodies (or on other constructs where an IgA Fc region is present or is the only Fc region) the ability (or enhance the ability or increase the ability) to bind to C1q.

The inventors have demonstrated herein (see Example 1) that the inclusion of both an IgA Fc region and an IgG Fc region in the constructs of the present invention can confer upon IgA antibodies (and therefore on other constructs where an IgA Fc region is present or is the only Fc region) the ability (or enhance the ability or increase the ability) to bind to C1q, e.g. compared to a full-length IgA antibody (e.g. a wild-type full-length IgA antibody) of the appropriate subtype selected for example depending on the subtype of the IgA Fc region included in the construct of the invention.

In addition, the inventors have demonstrated herein (see Example 1) that the inclusion of both an IgA Fc region and an IgG Fc region in the constructs of the present invention does not reduce the ability of said constructs, which comprise an IgG Fc region (or Fc fragment), to bind to C1q e.g. compared to a full-length IgG antibody (e.g. a wild-type full-length IgG antibody) of the appropriate subtype. Indeed, the inventors have shown that the ability to bind to C1q for the constructs of the invention is at least equivalent to the ability to bind to C1q of an appropriate full-length IgG antibody (e.g. a wild-type full-length IgG antibody), e.g. an IgG1 antibody, or an IgG2 antibody. Surprisingly and advantageously, the ability to bind to C1q of a construct of the present invention which comprises an IgG1 Fc region is shown to be improved or superior to the ability of a full-length IgG1 antibody to bind to C1q. This improvement, observed for the constructs of the invention with an IgG1 Fc region and an IgA Fc region as compared to a single IgG1 Fc region (e.g. in a full-length IgG1 antibody) could not have been predicted, and it is believed that this improved ability to bind to C1q should translate to an improved ability to induce CDC for protein constructs in accordance with the invention. Indeed, this is shown for protein constructs of the invention (see Example 1 and Example 2) where constructs show comparable or improved or increased ability to induce CDC for example as compared to an appropriate full-length IgG antibody (e.g. a wild-type full-length IgG antibody), e.g. an IgG1 antibody, or an IgG2 antibody. The inclusion of REW mutations as described elsewhere herein can improve the CDC induction even further. In addition, the extended constructs of the invention as described elsewhere herein and in Example 2, with or without REW mutations, also show such improved ability to induce CDC.

Also, the ability to bind to C1q of a construct of the present invention is shown to be improved or superior to the ability to bind to C1q of a construct with reverse orientation to that of the present invention, i.e. a construct in which the C-terminals of the IgG1 Fc region are connected to the N-terminals of the IgA Fc region. The ability of a construct of the present invention to induce CDC is shown to be improved or superior to the ability to induce CDC of a construct with reverse orientation to that of the present invention, i.e. a construct in which the C-terminals of the IgG1 Fc region are connected to the N-terminals of the IgA Fc region.

Thus, in preferred embodiments, the ability of a protein construct of the invention to bind C1q is maintained or not significantly altered (e.g. as compared to an IgG Fc containing control protein), or increased (preferably significantly increased) as compared to the ability of a control protein to bind C1q.

Thus, in preferred embodiments, the ability of a protein construct of the invention to induce CDC is maintained or not significantly altered (e.g. as compared to an IgG Fc containing control protein), or increased (preferably significantly increased) as compared to the ability of a control protein to induce CDC.

In some embodiments, the protein constructs of the invention are capable of inducing CDC of a range of cells, e.g. cancer cells depending on the targeting domain which is used, for example CD20 or HER2 expressing cells or cancer cells. Purely by way of example, and not meant to provide an exhaustive list, the protein constructs of the invention have been shown to be capable of inducing CDC of CD20 expressing cancer cells, e.g. cancerous B cell lines, including Raji cells, WSU-NHL cells or SU-DHI4 cells.

The induction of CDC can conveniently be measured by determining % lysis of appropriate target cells. The % lysis values will naturally vary depending on the cell type concerned. However, the exemplified constructs of the invention show good % lysis levels of cancer cells, for example levels of at least or up to 40% lysis are seen for different types of cancer cells, with levels of at least or up to 45%, 50%, 55%, 60%, 65%, 70%, 75%, 80% or 85% being observed. Purely by way of example, protein constructs of the invention which have a CD20 targeting domain can induce at least, or up to, 40%, 45%, 50%, 55%, 60%, 65%, 70%, 75%, 80% or 85% lysis of Raji cells (a CD20 expressing cancerous cell line).

In some embodiments, the ability of a protein construct of the invention to bind to C1q (or induce CDC) is increased, for example, by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold or at least 10-fold, as compared to the ability of a control protein to bind to C1q (e.g. there is an up to 1.1-fold, up to 1.2-fold, up to 1.3-fold, up to 1.4-fold, up to 1.5-fold, up to 1.6-fold, up to 1.7-fold, up to 1.8-fold, up to 1.9-fold, up to 2-fold, up to 3-fold, up to 4-fold, up to 5-fold, up to 6-fold, up to 7-fold, up to 8-fold, up to 9-fold, up to 10-fold, up to 15-fold or up to 20-fold increase, such as a 1.2-fold to 20-fold increase, 1.2-fold to 10-fold, 1.2-fold to 8-fold, 1.3-fold to 20-fold increase, 1.3-fold to 10-fold increase, 1.3-fold to 8-fold increase).

Where an increase in ability to bind C1q (or induce CDC) is achieved an appropriate control protein comprises an IgA Fc region, e.g. is an IgA antibody, e.g. a full-length IgA antibody (e.g. a wild-type full-length IgA antibody) of the appropriate sub-type such as an IgA1 or IgA2 antibody selected for example depending on the subtype of the IgA Fc region included in the construct of the invention, or another appropriate construct that contains a single IgA Fc region, e.g. contains a single IgA Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgG Fc region in accordance with the invention. In some embodiments, the protein construct of the invention comprises an IgA antibody and an exemplary control protein comprises the same IgA antibody with the sole difference (as compared to the protein of the invention) being that the control protein does not comprise an IgG Fc region in accordance with the invention.

Alternatively, where an increase in ability to bind C1q (or induce CDC) is achieved, or wherein a maintenance of ability to bind C1q (or induce CDC) is observed, e.g, wherein the ability to bind C1q (or induce CDC) is not significantly altered (or not altered) or is substantially equivalent to (or equivalent to or comparable to), the ability of a control protein to bind C1q (or induce CDC), an appropriate control protein may comprise an IgG Fc region, e.g. an IgG antibody, e.g. a full-length IgG antibody (e.g. a wild-type full-length IgG antibody) of the appropriate sub-type such as an IgG1, IgG2, IgG3 or IgG4 antibody selected for example depending on the subtype of the IgG Fc region included in the construct of the invention, or another appropriate construct that contains a single IgG Fc region, e.g. contains a single IgG Fc region which is the only Fc region in the construct. Preferably, such a control protein is substantially identical to (or identical to) or equivalent to the protein construct of the invention with which the comparison is being made, with the exception (or difference) being that the control protein construct does not comprise an IgA Fc region in accordance with the invention.

Appropriate control proteins will preferably contain the same or equivalent targeting domain or antigen binding domain as the protein construct of the invention.

A level of binding to C1q so as to be functionally effective, e.g. at a level to allow the induction of CDC activity is desired. The ability of a protein construct of the invention to bind to C1q may be assessed by any appropriate method or assay and the skilled person is familiar with appropriate assays. The discussion above may be in relation to C1q binding as determined by (or as assessed by) any appropriate assay. Typically, and preferably, an ELISA assay is used. A particularly preferred assay for determining C1q binding of protein constructs of the invention is described in Example 1 herein. Similarly, the ability of a protein construct of the invention to induce CDC may be assessed by any appropriate method or assay and the skilled person is familiar with appropriate assays. The discussion above may be in relation to the ability to induce CDC as determined by (or as assessed by) any appropriate assay. Typically, and preferably, an assay in which CDC induced lysis of target cells (i.e. cells expressing target antigen that is recognized by the constructs of the invention is used) can be measured is used, e.g. a chromium release assay or a fluorescence release assay such as a Calcein AM-based CDC assay. Examples of appropriate cells, e.g. cancer cells, e.g. cancerous B cells, are as described in the Examples herein and include Raji cells, WSU-NHL cells or SU-DHI4 cells. A particularly preferred assay for determining CDC activity of protein constructs of the invention is described in Example 1 herein.

Another important effector function that some antibodies have is the ability to induce ADCC. The ability of an antibody or other appropriate protein construct to induce ADCC is generally mediated by binding of an Fc region of the antibody or construct to an appropriate FcR on an effector cell. This interaction can then trigger a signalling cascade in the effector cells which results in the secretion of various factors and eventually the destruction of the target cell to which the antibodies (or constructs) are bound. In general, the ability and the potency to induce ADCC varies with the Fc region concerned and whether or not (or how well) the Fc region can bind to FcRs on effector cells. The induction of ADCC can also depend on the ratio between activating and inhibitory receptors. For example, IgG antibodies can induce ADCC via binding to the FcγRs on effector cells such as natural killer (NK) cells, macrophages, monocytes and eosinophils). Wild-type human IgG1 Fc and IgG3 Fc regions generally have good (high) ability to induce ADCC, and IgG2 Fc regions or IgG4 Fc regions generally have a weaker or lower ability to do this. On the other hand, IgA antibodies (e.g. IgA1 and IgA2 antibodies) generally have good (high) ability to induce ADCC via binding to FcαRs (FcαRI) on polymorphonuclear leukocytes (PMNs), mainly neutrophils.

Thus, in preferred embodiments, protein constructs in accordance with the present invention have or retain the ability to induce antibody dependent cellular cytotoxicity (ADCC). Thus, in preferred embodiments, the ability of a protein construct of the invention to induce ADCC is maintained or not significantly altered or not significantly reduced (e.g. as compared to an appropriate Fc containing control protein, e.g. a full-length IgA or IgG (e.g. a wild-type full-length IgA or IgG antibody) of the appropriate sub-type, e.g. selected depending on the sub-type of the IgA and/or IgG Fc regions included in the constructs of the invention), or the ability to induce ADCC is increased (preferably significantly increased), e.g. as compared to the ability of an appropriate Fc containing control protein (e.g. a full-length IgA or IgG, e.g. a wild-type full-length IgA or IgG antibody, of the appropriate sub-type, e.g. selected depending on the sub-type of the IgA and/or IgG Fc regions included in the constructs of the invention) to induce ADCC.

For constructs of the present invention that comprise an IgA Fc region, preferably said constructs can induce ADCC through engagement of neutrophils. For constructs of the present invention that comprise an IgG Fc region, preferably said constructs can induce ADCC through engagement of NK cells. Thus, preferred constructs of the present invention that comprise both an IgG Fc region and an IgA Fc region, preferably can induce ADCC through engagement of both neutrophils and NK cells.

Significant levels of ADCC activity are maintained or retained or present in the constructs of the present invention. For example, a level of ADCC so as to be functionally effective, e.g. at a level to allow the killing of appropriate target cells is desired. The ability of a protein construct of the invention to induce ADCC may be assessed by any appropriate method or assay and the skilled person is familiar with appropriate assays. The discussion above may be in relation to ADCC as determined by (or as assessed by) any appropriate assay. Typically, and preferably, an assay in which ADCC induced lysis of target cells (i.e. cells expressing target antigen that is recognized by the constructs of the invention is used) can be measured is used, e.g. a chromium release assay. Typically, and preferably, ADCC activity may be expressed in terms of the amount of specific lysis or specific cell lysis (e.g. % specific lysis or % specific cell lysis).

Another important effector function that some antibodies have is the ability to induce ADCP. The ability of an antibody or other appropriate protein construct to induce ADCP is generally mediated by binding of an Fc region of the antibody or construct to an appropriate FcR on an effector cell, where the effector cell is in turn capable of phagocytosis and therefore the destruction of the target cell to which the antibodies (or constructs) are bound. In general, the ability and the potency to induce ADCP varies with the Fc region concerned and whether or not (or how well) the Fc region can bind to FcRs on effector cells that are capable of phagocytosis. For example, IgG antibodies can induce ADCP via binding to the FcγRs on effector cells such as macrophages. Wild-type human IgG1 Fc and IgG2 regions generally have good (high) ability to induce ADCP. On the other hand, IgA antibodies (e.g. IgA1 and IgA2 antibodies) only show an intermediate ability to induce ADCP via binding to FcαRs (FcαRI) on appropriate effector cells such as macrophages.

Thus, in preferred embodiments, protein constructs in accordance with the present invention have or retain the ability to induce antibody-dependent cellular phagocytosis (ADCP). Thus, in preferred embodiments, the ability of a protein construct of the invention to induce ADCP is maintained or not significantly altered or not significantly reduced (e.g. as compared to an appropriate Fc containing control protein, e.g. a full-length IgA or IgG (e.g. a wild-type full-length IgA or IgG antibody) of the appropriate sub-type, e.g. selected depending on the sub-type of the IgA and/or IgG Fc regions included in the constructs of the invention), or the ability to induce ADCP is increased (preferably significantly increased), e.g. as compared to the ability of an appropriate Fc containing control protein (e.g. a full-length IgA or IgG, e.g. a wild-type full-length IgA or IgG antibody, of the appropriate sub-type, e.g. selected depending on the sub-type of the IgA and/or IgG Fc regions included in the constructs of the invention) to induce ADCP.

Significant levels of ADCP activity are maintained or retained or present in the constructs of the present invention. For example, a level of ADCP so as to be functionally effective, e.g. at a level to allow the phagocytosis of appropriate target cells is desired. The ability of a protein construct of the invention to induce ADCP may be assessed by any appropriate method or assay and the skilled person is familiar with appropriate assays. The discussion above may be in relation to ADCP as determined by (or as assessed by) any appropriate assay. Typically, and preferably, an assay in which ADCP induced phagocytosis of target cells (i.e. cells expressing target antigen that is recognized by the constructs of the invention is used) can be measured is used, e.g. assays in which macrophages are used to induce phagocytosis of target cells.

Preferably, the protein constructs of the invention comprising an IgA Fc region connected to an IgG Fc region have one or more, and preferably all of the functional properties described herein.

Preferably, the above described abilities and properties are observed at a measurable or significant level and more preferably at a statistically significant level, when compared to appropriate controls (e.g. control proteins). In any statistical analysis referred to herein, preferably the statistically significant difference over a relevant control or other comparative entity or measurement has a probability value of <0.1, preferably <0.05 (or ≤0.05). Appropriate methods of determining statistical significance are well known and documented in the art and any of these may be used.

In the protein constructs of the present invention the C-terminals of the IgA Fc region are connected to the N-terminals of the IgG Fc region. Such connection can be by any convenient means, for example can be direct, e.g. with no intermediate entity, or indirect, e.g. via an intermediate entity such as a linker, or a protein or polypeptide sequence, unit or domain, e.g. a structural sequence, unit or domain, e.g. a CH1 domain as discussed elsewhere herein. Thus, the IgA Fc region and the IgG Fc region can be attached to or linked to each other in any appropriate way such that each Fc region can still carry out their function. In some embodiments of the invention the protein constructs will contain linkers (physical linkers or linker molecules) between different parts of the construct, e.g. to connect the IgA Fc region and the IgG Fc region, and/or to connect the IgA Fc region to other parts of the construct such as a targeting domain or antigen binding domain as described elsewhere herein.

Thus, in preferred constructs of the invention the C-terminals of the IgA Fc region are connected to the N-terminals of the IgG Fc region by linkers. Any appropriate linker molecules can be used which would be well known to a person skilled in the art. For example, peptide (or polypeptide) linkers or chemical linkers or other covalent linkers can be used as appropriate.

Peptide or protein (polypeptide) linkers are generally preferred. Such peptide linkers, which may comprise non-natural or natural amino acids, or may comprise native or non-native (e.g. synthetic) sequences, are well known in the art, and appropriate linkers with an appropriate sequence, length, and/or flexibility/rigidity, can thus readily be selected by a skilled person in order to allow the various components of the protein constructs of the invention to be connected together in a stable way but with the correct spatial orientation or spatial optimisation so that the above required functional properties (i.e. the functional properties of each component, e.g. the IgA Fc region and the IgG Fc region, or the function of the targeting domain or antigen binding domain as described elsewhere herein) are retained once the individual components are attached or connected to each other.

Thus, the linker or spacer can aid the folding of the connected proteins, and the spacer or linker length and/or flexibility/rigidity, can be adjusted as appropriate to enable the best or satisfactory functional folding of each component. Appropriate lengths could readily be determined by a person skilled in the art and could be any appropriate number of amino acids. However, exemplary lengths might be at least 5, 10, 15, 20, 25, 30, 35, 40, or 45 amino acids long (e.g. at least 6, 7, 8, or 9 amino acids long, or at least 11, 12, 13, or 14 amino acids long), or be between 5 or 10 and 50 or 60 or 70 amino acids, e.g. 5 or 10 to 15, 20, 25, 30, 35, 40, 45, 50, 60 or 70 amino acids, or 15 to 20, 25, 30, 35, 40, 45, 50, 60 or 70 amino acids, or 20 to 25, 30, 35, 40, 45, 50, 60 or 70 amino acids, or 25 to 30, 35, 40, 45, 50, 60 or 70 amino acids, or 30 to 35, 40, 45, 50, 60 or 70 amino acids. Preferred linkers can be 15 to 30 amino acids long, e.g. can be or be up to 15, 20, 25 or 30 amino acids long (or up to 40 or 50 or 60 or 70 amino acids long).

Some constructs of the invention are described herein as extended constructs as the tandem IgA Fc regions and IgG Fc regions in these constructs are further separated from each other than in the non-extended constructs of the invention. As described elsewhere herein, in one specific embodiment this is achieved by the inclusion of a CH1 domain (or an alternative structural protein sequence, unit or domain) and optionally one or more further linkers, e.g. as described herein, such as antibody hinge regions. However, in other embodiments this can be achieved by the inclusion of longer (extended) linkers or spacers to those described above. Thus, in such embodiments, these linkers between (or which connect) the tandem IgA Fc regions and the IgG Fc regions in the constructs of the invention can be, or can be at least, or can be up to 80, 90, 100, 105, 110, 115, 120, 125, 128, 129, 130, 135, 140, 145, or 150 amino acids long, e.g. 90, 100 or 120, to 150 amino acids long.

As outlined above, appropriate linkers may comprise (or consist of) native or non-native (e.g. synthetic) sequences. Exemplary non-native or synthetic linkers are described in the art and can include linkers which comprise (or consist of) glycine and/or serine residues, e.g. GS linkers, which may contain one or more repeats of GS sequences such as one or more repeats of the G4S linker (GGGGS (SEQ ID NO: 44)).

Exemplary native or naturally occurring peptide or polypeptide sequences that can be used as linkers would also be well known to a person skilled in the art. For example, conveniently and preferably the peptide (or polypeptide) linkers used in the protein constructs of the invention to connect the C-terminals of the IgA Fc region to the N-terminals of the human IgG Fc region comprise an antibody hinge region. The sequences of such hinge regions are well known and documented in the art and exemplary sequences are shown in Table 1. For example, appropriate hinge regions comprise an IgA antibody hinge region or an IgG antibody hinge region. As also explained elsewhere herein, in the constructs of the invention it is particularly convenient to use an IgG hinge region, for example an IgG hinge region from the IgG subtype (IgG1, IgG2, IgG3 or IgG4) being used for the IgG Fc region of the construct, to connect the C-terminals of the IgA Fc region to the N-terminals of the IgG Fc. All or part (e.g. a fragment) of such antibody hinge regions may be used and are included providing that the functional properties of the construct components are retained and preferably the functional properties of the hinge region are retained.

Selection of the nature of the linker and other properties such as appropriate linker lengths, for example to achieve the same (or similar) effects to those observed for the linkers, e.g. hinge regions, used in the exemplified constructs, would be a standard and routine procedure for a person skilled in the art. Preferred and exemplary lengths are provided elsewhere herein.

In other preferred embodiments of the invention, in particular in the extended constructs of the invention, the C-terminals of the IgA Fc region can be connected to the N-terminals of the IgG Fc region by a CH1 domain. In other words, a CH1 domain can be used as an intermediate entity to connect the IgA Fc region and the IgG Fc region. In such embodiments any CH1 domain can be used, and some examples are given in Table 1, e.g. IgG or IgA CH1 domains. In preferred embodiments the CH1 domains are IgG CH1 domains, for example IgG1 CH1 domains. Such preferred intermediate entities may comprise a CH1 domain and may also comprise other linker or spacer elements, e.g. as described above and elsewhere herein. For example, one or more linkers, e.g. peptide linkers, may be present, e.g. one or more antibody hinge regions. In such embodiments, the CH1 domain is located in between the IgA Fc region and the IgG Fc region in the tandem part of the construct and conveniently a linker can be used to connect the N-terminals of the CH1 domain to the C-terminals of the IgA Fc region and/or the C-terminals of the CH1 domain to the N-terminals of the IgG Fc region. Such linkers can be antibody hinge regions as described elsewhere herein. As in other embodiments, it is particularly convenient to use a hinge region which matches the nature of the CH1 domain. Thus, where the CH1 domain is an IgG CH1 domain then in some embodiments the linkers will be an IgG hinge region from the IgG subtype (IgG1, IgG2, IgG3 or IgG4) being used for the IgG CH1 domain. In preferred embodiments the CH1 domain is an IgG1 CH1 domain. In such and other embodiments a preferred linker is an IgG1 hinge region. A preferred such extended construct is shown in Example 2 and for example comprises, in the N-terminal to C-terminal direction, an IgA Fc region (e.g. IgA2), an IgG hinge region (e.g. an IgG1 hinge region), a CH1 domain (e.g. an IgG1 CH1 domain), a further IgG hinge region (e.g. an IgG1 hinge region), and an IgG Fc region (e.g. an IgG1 Fc region).

In such and other embodiments relating to extended constructs of the invention, a preferred overall length of the region of the construct which connects (is between) the C-terminals of the IgA Fc region to the N-terminals of the IgG Fc region is as described elsewhere herein for the linkers or spacers used in the constructs of the invention, e.g. at least or up to 150 amino acids long.

An advantage with using peptide (or polypeptide) linkers is that it enables production of the polypeptide chains of the constructs as single polypeptides, e.g. as fusion proteins or fusion polypeptides.

The term “fusion protein”, “fusion polypeptide”, etc., is used herein to describe the functional joining of two or more protein components in the same polypeptide sequence or in the same open reading frame (ORF). An example of such fusion proteins can also be described as genetic fusions as they are encoded by the same nucleic acid sequence (sometimes called a “fusion gene” or “fusion nucleotide sequence”). Although two (or more) protein components (or encoding nucleic acid sequences) can be directly adjacent to each other in such a fusion protein, equally and preferably the components can be joined by appropriate peptide or polypeptide spacers or linkers, e.g. as described above.

As the constructs of the present invention comprise two Fc dimers in tandem or adjacent to each other, in such embodiments, the protein construct conveniently comprises two fusion polypeptides (or fusion polypeptide chains). In such embodiments, each fusion polypeptide comprises (i) a polypeptide making up one chain of an IgA Fc region (i.e. one chain of the IgA Fc dimer) and (ii) a polypeptide making up one chain of an IgG Fc region (i.e. one chain of the IgG Fc dimer), wherein the polypeptide chain of the IgG Fc region is positioned (or located)C-terminally relative to the polypeptide chain of the IgA Fc region.

Thus, in such embodiments, the polypeptide chains (fusion polypeptides) typically comprise, from the N-terminal end to the C-terminal end, a polypeptide comprising one half of the IgA Fc dimer and a polypeptide comprising one half of the IgG Fc dimer. As will be described elsewhere herein the N-terminal ends of the polypeptides making up the IgA Fc region (or dimer) may also be connected (or fused) to other entities such as an appropriate targeting domain or antigen binding domain. Thus, the protein constructs of the invention (or the IgA Fc region and IgG Fc region components of the constructs) can be used to create Fc-fusions or Fc-fusion proteins with other appropriate entities. In other words, the connected IgA Fc region and IgG Fc region components of the constructs can provide a polypeptide unit which can for example be fused to any polypeptide or protein of interest.

The skilled person is familiar with methods of generating fusion polypeptides, e.g. by expressing a nucleic acid molecule encoding a fusion polypeptide (e.g. in a host cell). Such a nucleic acid molecule typically comprises a contiguous nucleotide sequence encoding, in frame, the various components of the fusion polypeptide. In the present case, such a nucleic acid molecule may comprise a contiguous nucleotide sequence comprising a nucleotide sequence encoding a polypeptide making up one chain of an IgA Fc region and a nucleotide sequence encoding a polypeptide making up one chain of an IgG Fc region, wherein the nucleotide sequence encoding the chain of the IgG Fc region is located at the 3′-end of the nucleic acid molecule encoding the chain of the IgA Fc region. Then, when expressed, dimerization can take place between the two polypeptide chains to form the complete IgA and IgG Fc regions (dimers).

Although the above discussion focuses on the presence of a linker or spacer between the IgA Fc and IgG Fc regions of the constructs, linker sequences may be included elsewhere in the constructs of the invention as appropriate, e.g. between other components of the constructs which may be present. Thus, in some constructs of the present invention linkers may also be included between the N-terminus or N-terminal end of the IgA Fc region and any other desired entity which is present in that position, such as a targeting domain or an antigen binding domain as described elsewhere herein. Such linkers can take the forms as described above, e.g. can be native or non-native (synthetic) peptide linkers. A convenient and preferred peptide (polypeptide) linker for this linkage can take the form of an antibody hinge region, conveniently an IgA antibody hinge region, for example an IgA hinge region from the IgA subtype (IgA1, IgA2) being used for the IgA Fc region of the construct. However, as described elsewhere herein, in other embodiments an IgG hinge region, e.g. an IgG1 hinge region, can be used. All or part (e.g. a fragment) of such antibody hinge regions may be used providing that the functional properties of the construct components are retained and preferably the functional properties of the hinge region are retained.

Although peptide (polypeptide) linkers are convenient and preferred for some aspects, any other appropriate means of attachment or connection might also be used, for example any other form of linker, including chemical linkers, e.g. chemical cross-linkers, providing that the functional properties (as discussed elsewhere herein) of the various components which are linked together are retained once the components are attached or connected to each other. Suitable cross-linking agents and methods for attaching (or connecting or joining or linking or conjugating) different proteins or polypeptides together are known in the art.

Thus, in some embodiments, the IgA Fc and IgG Fc regions of the constructs of the invention may be produced separately and then subsequently connected together (or attached together or joined together or linked together or conjugated together).

In some embodiments, no linker is present. Thus, for example, in some embodiments the polypeptides making up the IgA Fc region can be directly attached to the polypeptides making up the IgG Fc region in accordance with the invention. With such a direct attachment, in the context of a fusion polypeptide, the first (i.e. N-terminal) amino acid of the IgG Fc region may be fused directly to (via a peptide bond) the final (i.e. C-terminal) amino acid of the IgA Fc region (i.e. with no linker in between).

Any IgG Fc or IgA Fc regions can be used in the protein constructs of the present invention. Thus, in some embodiments these regions will comprise or correspond to wild-type or native sequences. However, in other embodiments, the IgG Fc region and/or the IgA Fc region can comprise mutations or modifications, in particular mutations or modifications which increase or enhance effector function of the Fc region or increase or enhance plasma half-life. Appropriate mutant or modified Fc regions are well known and described in the art and any of these may be used.

Preferred mutations or modifications are those which increase or enhance binding to Fc receptors, preferably increase or enhance binding to FcRn, or increase or enhance C1q binding.

In some embodiments, a preferred Fc region (IgG Fc region, e.g. IgG1, IgG2, IgG3 or IgG4 Fc region) for use in the constructs of the present invention is a modified IgG Fc region characterized by comprising one or more of the following modifications:
(i) an arginine (R) residue, or a similar residue such as a lysine (K) residue, at position 311 (or a position corresponding thereto);
(ii) a glutamic acid (E) residue, or a similar residue such as an aspartic acid (D) residue, at position 428 (or a position corresponding thereto); and
(iii) a tryptophan (W) residue, or a similar residue such as a tyrosine (Y) residue or a phenylalanine (F) residue, at position 434 (or a position corresponding thereto).

These positions are defined using the standard EU numbering for IgG regions in which the ASTK of the CH1 domain starts at position 118, see e.g. http://www.imgt.org/IMGTScientificChart/Numbering/Hu_IGHGnber.html #refs. Thus, the positions (or corresponding positions) of these residues in the various constructs of the invention can readily be determined. Indeed, these modified IgG Fc regions are also referred to herein as REW mutations and some exemplary sequences are shown in Table 1. The wild-type residues at positions 311, 428 and 434 are Q, M and N respectively for IgG1 and IgG2.

In preferred embodiments of the invention, two or more of the above modifications are present, most preferably all three of the above modifications. Such modifications are further described in WO2017/158426 and are examples of mutations or modifications that have the effect of increasing or enhancing the pH-dependent binding to FcRn. The inventors have also shown herein that such modifications can increase binding to C1q. An increased induction of CDC has also been shown. In some embodiments such modifications are preferred for use with IgG2 Fc regions. In some embodiments such modifications are preferred for use with IgG1 Fc regions. In some embodiments such modifications are preferred for use with IgG3 Fc regions, optionally together with the R435H mutation described below. In some embodiments such modifications are preferred for use with IgG4 Fc regions. In some embodiments REW substitutions are preferred.

Another preferred and possible additional modification which can be used in the IgG Fc regions in the constructs of the invention is the R435H mutation as described in Stapleton et al., 2011, Nat. Comm. 20 (2): 599. Such modifications also have the effect of increasing binding to FcRn and are preferred for use when the construct contains an IgG3 Fc region. Again this position is defined using the EU numbering system as described above.

In preferred embodiments, the constructs of the invention further comprise, e.g. are further connected to, a targeting domain (or targeting unit). Such targeting domains can be any entity that is capable of binding, e.g. specifically binding, to a desired target molecule or target entity. Thus, such targeting domains can be receptors, preferably receptor domains (e.g. receptors or receptor domains for ligands) or ligands (e.g. ligands for receptors or receptor ligands) or antigen binding domains. Preferred targeting domains and target molecules are proteins or polypeptides. Thus, preferred targeting domains are binding proteins.

In some embodiments, the targeting domain of the protein construct of the invention is or comprises an antigen binding domain.

Preferred antigen binding domains for use in the protein constructs of the invention are antibodies or antibody fragments, e.g. antigen binding fragments of antibodies.

Thus, in preferred embodiments of the present invention, the protein construct comprises an antibody (or immunoglobulin), or an antigen binding fragment thereof.

As the N-terminal region of the protein constructs of the invention comprises an IgA Fc region, particularly preferred antibodies are IgA antibodies, or antigen binding fragments thereof, in which case the Fc region is already provided in the construct. In some embodiments, the IgA antibody is an IgA1 antibody. In some embodiments, the IgA antibody is an IgA2 antibody. In other embodiments IgG antibodies, or antigen binding fragments thereof, are used as antigen binding domains. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody The subunit structures and three-dimensional configurations of different classes of antibodies are well known.

The terms “antibody” and “immunoglobulin”, as used herein, refer broadly to any immunological binding agent that comprises an antigen binding domain. This term includes antibody fragments that comprise an antigen binding domain.

As will be understood by those in the art, the immunological binding reagents encompassed by the term “antibody” includes or extends to all antibodies and antigen binding fragments thereof, including whole antibodies (or full-length antibodies), dimeric, trimeric and multimeric antibodies; bispecific antibodies; chimeric antibodies; recombinant and engineered antibodies, and fragments thereof.

Techniques for preparing and using various antibody-based constructs and fragments are well known in the art. Monoclonal antibodies are particularly preferred.

Preferably, the antibody or antibody fragment comprises an antibody light chain variable region (V_{L}) that comprises three CDR domains and an antibody heavy chain variable region (V_{H}) that comprises three CDR domains. Collectively, the six CDRs generally confer antigen-binding specificity to the antibody, although single domain antibodies with only 3 CDRs can equally be used, and it is also possible for antigen binding to be mediated by even one or two CDR regions, especially if only a low or moderate affinity binding is desired. The heavy and light chain variable regions also have four framework regions (FR1, FR2, FR3 and FR4 from the amino terminus to carboxy terminus). These framework regions separate the CDRs. Said VL and VH generally form the antigen binding site, although single domain antibodies can also be used which generally comprise only three CDR domains, e.g. comprise a VL or a VH region that can bind to antigen.

The CDRs of the antibodies used in the constructs of the invention are preferably separated by appropriate framework regions such as those found in naturally occurring antibodies and/or effective engineered antibodies. Thus, the CDR sequences are preferably provided within or incorporated into an appropriate framework or scaffold to enable antigen binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used.

As well as appropriate variable domains comprising CDR sequences and FR sequences which make up the antigen binding site, antibodies or antibody fragments for use in the constructs of the invention may also comprise antibody constant regions, e.g. heavy chain and/or light chain constant regions. The constant region of a heavy chain comprises three constant domains CH1, CH2 and CH3, and the constant region of a light chain comprises a CL constant domain. In some embodiments of the invention the antibody (or fragment) will further comprise a CH1 domain and/or a CL domain. In preferred embodiments these domains will be derived from an IgA antibody. Thus, in preferred constructs of the invention, as well as the IgA Fc region, and the VL and/or VH regions, CH1 and CL regions are provided, preferably CH1 and CL regions corresponding to or derived from an IgA antibody. In other preferred embodiments these domains will be derived from an IgG antibody. Thus, in preferred constructs of the invention, as well as the IgA Fc region, and the VL and/or VH regions, CH1 and CL regions are provided, preferably CH1 and CL regions corresponding to or derived from an IgG antibody.

Appropriate sequences for such constant regions are well known and documented in the art.

When a full complement of constant regions from the heavy and light chains is included in the constructs of the invention, such constructs are typically referred to as comprising “full-length” antibodies or “whole” antibodies. In preferred embodiments, the protein construct comprises a whole (or full-length) antibody, preferably a whole (or full-length) IgA antibody. “Whole” or “full-length” antibodies comprising two heavy chains and two light chains are preferred in some embodiments. Where a full-length IgA antibody is included then the IgA Fc region of the full-length IgA antibody can provide the IgA Fc region of the claimed construct.

However, in some embodiments, the antigen binding domain of the protein construct does not comprise a whole or full-length antibody. In some embodiments, the protein construct comprises an antigen binding fragment of an antibody, preferably a fragment of an IgA antibody. In other preferred embodiments a fragment of an IgG antibody can be used. Such an antigen binding fragment may comprise three or six CDRs, for example be a sdAb or an scFv or Fv antibody, and may comprise a CH1 and/or a CL region, for example be a Fab fragment. Appropriate antigen binding fragments of antibodies and formats are known in the art and any of these may be used. Conveniently the protein constructs of the invention comprise two antigen binding domains (or other targeting domains), one attached or connected to each chain of the IgA Fc region/fragment, for example the antigen binding domain can take the form of a Fab2 fragment. Indeed, the use of an IgG, for example an IgG1, Fab2 fragment is preferred in some embodiments, for example in the extended constructs of the invention as described herein, for example when also combined with the presence of a CH1 domain (or an alternative structural protein sequence, unit or domain), or an extended linker sequence, as an intermediate entity to connect the IgA Fc region and the IgG Fc region of the constructs of the invention as described elsewhere herein. These two antigen binding domains (or other targeting domains) can be the same or different. Equally only one antigen binding domain (or other targeting domain) can be used, e.g. connected to only one of the chains of the IgA Fc region/fragment.

In some embodiments where the protein constructs comprise an antigen binding fragment or targeting domain which is made up of a single polypeptide chain, e.g. an sdAb (e.g. a nanobody or V_{H}H antibody, or VH antibody or VL antibody), or an scFv fragment, then the protein constructs of the invention can comprise (or consist of) 2 polypeptide chains.

In some embodiments where the protein constructs comprise an antigen binding fragment or targeting domain which is made up of two polypeptide chains, e.g. a Fab fragment or for example is provided by a full-length antibody, then the protein constructs of the invention can comprise (or consist of) 4 polypeptide chains.

Thus, in accordance with the present invention, the protein construct comprises (or consists of) two polypeptide chains which make up (or form) the IgA Fc and IgG Fc regions, e.g. the tandem, adjacent or fused Fc regions present in the constructs of the invention.

In some embodiments, the antibody (or antigen binding fragment) for use in the constructs of the invention has been reformatted (e.g. from an IgG antibody or other class of antibody) into the IgA format (e.g. the IgA1 or IgA2 format). Thus, in some embodiments, the antibody (or antigen binding fragment) is an IgA antibody comprising an antigen binding domain of a non-IgA antibody (e.g. from or derived from or based upon an antigen binding domain of a non-IgA antibody, e.g. of an IgG antibody). Methods of reformatting antibodies into the IgA format are well-known in the art and the skilled person will be familiar with such methods.

In some embodiments, the IgA antibody (or antigen binding fragment thereof) for use in the constructs of the invention is modified (or mutated) in order to keep it in monomeric form (e.g. to prevent it dimerizing). Appropriate modifications in this regard include the IgA antibody having a modified (or mutated or inactivated or truncated) tailpiece, or not comprising a tailpiece (or where the tailpiece has been removed) as described elsewhere herein.

In preferred embodiments, the sequences making up the targeting domains, e.g. the receptors, receptor domains, ligands, receptor ligands, antigen binding domains, antibodies (or antigen binding fragments thereof) for use in the constructs of the invention are human sequences. In this regard, human sequences, e.g. human antibodies, generally have potential advantages for use in human therapy, for example the human immune system should not recognize the antibody as foreign.

In other preferred embodiments, and as indicated elsewhere herein, preferred IgA Fc regions and/or IgG Fc regions are human Fc regions.

The term “human” as used herein in connection with antibody molecules refers to antibodies having variable regions (e.g., VH, VL, CDR or FR regions) and, preferably, constant antibody regions, isolated or derived from a human repertoire or derived from or corresponding to sequences found in humans or a human repertoire, e.g., in the human germline or somatic cells or bodily fluids, or in a human antibody library such as a phage display library. Similarly, the term “human” as used herein in connection with targeting domains (or binding proteins) or Fc regions refers to protein sequences isolated or derived from a human or corresponding to sequences found in humans, e.g., in the human germline or somatic cells. Thus, such human sequences can obtained from human samples, e.g. from human bodily fluids or human cells. Humanized sequences, e.g. humanized targeting domains, antigen binding domains, antibodies or antibody fragments, or humanized Fc regions can also be used in the constructs.

Non-antibody or non-immunoglobulin based targeting domains or binding proteins can also be used in the constructs of the invention and can be selected for the ability to specifically bind to a particular target molecule or target antigen in their own right. Such molecules are also referred to as antibody mimics (or antibody mimetics). Examples of appropriate non-immunoglobulin based targeting domains or binding proteins are known and described in the art and include fibronectins (or fibronectin-based molecules), for example based on the tenth module of the fibronectin type III domain, such as Adnectins (e.g. from Compound Therapeutics, Inc., Waltham, MA); affimers (e.g. from Avacta); ankyrin repeat proteins (e.g. from Molecular Partners AG, Zurich, Switzerland); lipocalins, e.g. anticalins (e.g. from Pieris Proteolab AG, Freising, Germany); human A-domains (e.g. Avimers); staphylococcal Protein A (e.g. from Affibody AG, Sweden); thioredoxins; and gamma-B-crystallin or ubiquitin based molecules, e.g. affilins (e.g. from SciI Proteins GmbH, Halle, Germany). As mentioned above, such molecules can also be used as scaffolds onto which appropriate CDRs which mediate target antigen binding can be grafted. For example, the CDRs of an appropriate immunoglobulin based targeting domain or binding protein can be grafted onto an appropriate non-immunoglobulin scaffold.

Generally said targeting domains are located at the N-terminal end or in the N-terminal region of the constructs of the invention. Thus, in preferred embodiments, said targeting domains are connected to the N-terminal side of the IgA Fc region of the construct, for example are connected to the N-terminus or N-terminal end of the IgA Fc region. Such connection can be direct or indirect, e.g. via a linker, preferably an antibody hinge region, as described elsewhere herein. It is of course important that the ability of the targeting domain to bind to its target is retained (or not significantly affected) when incorporated into the constructs of the invention.

Preferred target molecules to be bound by the targeting domains are therapeutically (or clinically) relevant target proteins (or target antigens). Thus, in some embodiments, the protein constructs of the present invention bind to a disease-associated target protein (or target antigen) by virtue of the targeting domains. A disease-associated target protein (or target antigen) may be a target protein (or antigen) whose expression (e.g. unwanted expression or aberrant expression or overexpression) is associated with a disease. In some embodiments, the disease is cancer (or a tumour), for example a solid tumour such as a breast cancer, or a haematological cancer. Alternatively said disease is caused by a pathogen, e.g. an infectious pathogen such as bacteria.

Thus, in some embodiments, said targeting domain binds to a given cancer (or tumour) protein or antigen (e.g. a cancer specific protein or antigen, or a cancer associated protein or antigen, or a tumour specific protein or antigen, or a tumour associated protein or antigen). In some embodiments the cancer is breast cancer. In other embodiments said targeting domain binds to a protein or antigen associated with an infectious agent, e.g. a bacteria.

Thus, in some embodiments the targeting domain, e.g. the antigen binding domain, binds to a target molecule on a cancer cell or on an infectious pathogen. Preferably said cancer cell is from a solid tumor or a haematological cancer, or said infectious pathogen is a bacteria. In some embodiments the cancer or solid tumour is breast cancer.

An exemplary cancer associated target is a member of the human epidermal growth factor receptor. Thus, in some embodiments, constructs of the present invention, e.g. constructs comprising a targeting domain, e.g. an antigen binding domain, bind to HER2. Over-expression of the protein HER2 can play an important role in the development and progression of certain breast cancers.

In some embodiments, constructs of the present invention comprise the antigen binding domain of an anti-HER2 antibody, e.g. comprise the antibody Trastuzumab.

A further exemplary cancer associated target, e.g. associated with cancerous B cell lines and therefore appropriate for treatment of haematological cancers is CD20. Thus, in some embodiments, constructs of the present invention, e.g. constructs comprising a targeting domain, e.g. an antigen binding domain, bind to CD20. Preferred constructs thus comprise anti-CD20 antibodies or antigen binding fragments thereof. Over-expression of the CD20 protein can play an important role in the development and progression of certain haematological cancers, in particular cancers involving B cells.

In another aspect, the present invention provides a method of extending the in vivo half-life (e.g. plasma or serum half-life) of a protein (preferably an IgA antibody or fragment thereof, or an alternative targeting domain as described herein), said method comprising incorporating said protein, etc., into a protein construct of the invention (preferably at the N-terminal end of said construct, e.g. connected to the N-terminals of the IgA Fc region), or attaching a protein construct of the invention (or attaching a polypeptide unit as described above) to said protein, etc., (preferably to the C-terminal end of said protein). Discussion of various features of the proteins and protein constructs of other aspects of the invention and preferred embodiments apply mutatis mutandis to this aspect of the invention.

The Fc regions for use in the constructs of the present invention can be obtained from or be derived from or can correspond to Fc regions from any source or species, or can be a fragment or variant thereof providing that the ability to bind Fc receptors or to induce effector function is retained. Preferred sources or species are mammalian, and any appropriate mammalian source or species may be used, for example humans or any livestock, domestic or laboratory animal. Specific examples include mice, rats, pigs, cats, dogs, sheep, rabbits, horses, cows and non-human primates (e.g. cynomolgus monkey). Preferably, however, the mammal is a human. Sequences of Fc regions from various species are known in the art and thus appropriate Fc regions for use in the invention can be readily generated or produced by standard techniques, e.g. recombinant techniques.

Although the above discussion is focussed on describing the protein constructs of the invention, conveniently said protein constructs will be prepared or produced using appropriate nucleic acid molecules encoding all or part of such protein constructs.

Thus, it can be seen that nucleic acid molecules, e.g. one or more nucleic acid molecules (e.g. a set of nucleic acid molecules), comprising nucleotide sequences that encode the protein constructs of the present invention, e.g. the recombinant protein constructs as defined herein, or parts (for example single chains or the first or second chains of the protein constructs) form yet further aspects of the invention. Expression vectors comprising such nucleic acid molecules, e.g. one or more nucleic acid molecules, and host cells comprising said expression vectors or nucleic acid molecules or protein constructs form yet further aspects.

Typically, the one or more nucleic acid fragments encoding the protein constructs of the invention are incorporated into one or more appropriate expression vectors in order to facilitate production of the protein constructs, e.g. the recombinant protein constructs, of the invention.

The invention therefore contemplates an expression vector, e.g. one or more expression vectors, e.g. one or more recombinant expression vectors, containing or comprising one or more (or a set of) nucleic acid molecules of the invention, and the necessary regulatory sequences for the transcription and translation of the protein sequence encoded by the nucleic acid molecules of the invention. The vectors may also contain sequences to enable antibiotic resistance and replication of the vector. Suitable vectors and regulatory sequences would be well known to a person skilled in the art.

Expression vectors, e.g. recombinant expression vectors, of the invention, or nucleic acid molecules of the invention, can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells. For example, the molecules of the invention may be expressed in yeast cells, or mammalian cells, or prokaryotic cells such as Escherichia coli or Pichia pastoris.

The protein constructs and nucleic acid molecules of the invention are generally “isolated” or “purified”. The term “isolated” or “purified” typically refers to a protein or nucleic acid that is substantially free of cellular material or other proteins (or other nucleic acids) from the source from which it is derived or produced.

A person skilled in the art will appreciate that the protein constructs of the invention may be prepared in any of several ways well known and described in the art, but are most preferably prepared using recombinant methods. For example, the various components of the constructs can be encoded on a single polypeptide chain or multiple polypeptide chains, as appropriate, after which the various components are then joined or linked together, or the multiple polypeptide chains otherwise associate with each other to form the protein constructs of the invention.

Thus, a yet further aspect of the invention provides a method of producing the protein constructs of the invention, comprising a step of culturing the host cells of the invention. Preferred methods comprise the steps of (i) culturing a host cell comprising one or more of the expression vectors or one or more of the nucleic acid sequences of the invention under conditions suitable for the expression of the encoded protein construct; and optionally (ii) isolating or obtaining the expressed protein construct from the host cell or from the growth medium/supernatant. Such methods of production may also comprise a step of purification of the protein product and/or formulating the protein product into a composition including at least one additional component, such as a pharmaceutically acceptable carrier or excipient.

As the preferred protein constructs of the invention generally comprise two identical polypeptide chains (e.g. where a targeting domain is not present or is a single polypeptide chain such as a single chain antibody) or a pair of identical polypeptide chains (e.g. where the targeting domain is made up of two polypeptide chains such as an antibody or antibody fragment with a separate heavy and light chain), then, in such embodiments, a single or two polypeptide chains, as appropriate, are expressed in the host cell, so that the complete protein constructs of the invention can assemble in the host cell and be isolated or purified therefrom. The protein constructs of the invention can be produced, purified or isolated by standard methods which would be well known to a person skilled in the art.

The invention also provides a range of conjugated proteins and fragments thereof in which the protein construct of the invention is operatively attached to at least one other agent (e.g. a therapeutic agent) that is distinct from any targeting domain that is present in the constructs of the invention. The term “immunoconjugate” is broadly used to define the operative association of the protein construct with another effective agent (e.g. a therapeutic agent). Recombinant fusion proteins are particularly contemplated. So long as the targeting domain is able to bind to the target and the other agent is sufficiently functional upon delivery, the mode of attachment will be suitable.

In some embodiments, protein constructs of the invention are used (e.g. used therapeutically) in their “naked” non-immunoconjugated form, e.g. in a form comprising a targeting domain together with the tandem IgA Fc region and IgG Fc region.

Compositions comprising a protein construct of the invention (or one or more nucleic acid molecules or expression vectors of the invention or immunoconjugates of the invention) constitute yet further aspects of the present invention. Formulations (compositions) comprising one or more protein constructs of the invention (or one or more nucleic acids or expression vectors of the invention or immunoconjugates of the invention) in a mixture with a suitable diluent, carrier or excipient constitute a preferred embodiment of the present invention. Such formulations may be for pharmaceutical use (are pharmaceutical compositions) and thus compositions of the invention are preferably pharmaceutically acceptable compositions. Suitable diluents, excipients and carriers are known to the skilled man.

The compositions according to the invention may be presented, for example, in a form suitable for oral, nasal, parenteral, intravenal, topical or rectal administration. Unless otherwise stated, administration is typically by a parenteral route, preferably by injection subcutaneously, intramuscularly, intracapsularly, intrathecally, intraperitoneally, intratumouraly, transdermally or intravenously. In some embodiments subcutaneous administration is preferred.

The compositions according to the invention defined herein may be presented in the conventional pharmacological forms of administration, such as coated tablets, nasal or pulmonal sprays, solutions, liposomes, powders, capsules or sustained release forms. Conventional pharmaceutical excipients as well as the usual methods of production may be employed for the preparation of these forms.

Injection solutions may, for example, be produced in the conventional manner, such as by the addition of suitable preservation agents or stabilizers. The solutions are then filled into injection vials or ampoules.

Nasal sprays may be formulated similarly in aqueous solution and packed into spray containers, either with an aerosol propellant or provided with means for manual compression.

Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the molecule or protein construct in the form of a nasal or pulmonal spray. As a still further option, the molecules or protein constructs of the invention can also be administered transdermally, e.g. from a patch, optionally an iontophoretic patch, or transmucosally, e.g. bucally.

Suitable dosage units can be determined by a person skilled in the art.

The pharmaceutical compositions may additionally comprise further active ingredients (e.g. as described elsewhere herein) in the context of co-administration regimens or combined regimens.

The protein constructs of the invention as defined herein may be used as molecular tools for in vitro or in vivo applications and assays. Thus, yet further aspects of the invention provide a reagent that comprises a protein construct (or other molecule) of the invention as defined herein and the use of such protein constructs (or other molecules) as molecular tools, for example in in vitro or in vivo assays.

A further aspect of the present invention provides protein constructs of the invention (e.g. constructs comprising targeting domains, e.g. constructs comprising IgA antibodies or fragments thereof) for use in therapy. Therapy includes treatment (e.g. of pre-existing disease) or prevention (prophylaxis). Active treatment of pre-existing disease is preferred in some embodiments.

In some embodiments, the present invention provides protein constructs of the invention that comprise a targeting domain, e.g. a receptor, receptor domain, ligand, receptor ligand, or an antigen binding domain (e.g. constructs comprising IgA or IgG antibodies or fragments thereof) that binds to a given target or antigen, for use in the treatment of a disease that is characterized by (or associated with) the expression of said target or antigen (e.g. unwanted or aberrant expression of said target or antigen), for example on the cell surface.

For example, in some embodiments, the invention provides protein constructs of the invention that comprise a targeting domain, e.g. a receptor, ligand, or an antigen binding domain (e.g. constructs comprising IgA or IgG antibodies or fragments thereof) that binds to a given cancer (or tumour) antigen (e.g. cancer specific antigen or cancer associated antigen or tumour specific antigen or tumour associated antigen) for use in the treatment of cancer (or a tumour). In some embodiments, the cancer or tumour antigen is CD20 and the targeting domain used is one that binds to CD20.

For example, in some embodiments, the invention provides protein constructs of the invention that comprise a targeting domain, e.g. a receptor, receptor domain, ligand, receptor ligand, or an antigen binding domain (e.g. constructs comprising IgA or IgG antibodies or fragments thereof) that binds to a given molecule or antigen expressed by a pathogen (e.g. a pathogen specific antigen or pathogen associated antigen) for use in the treatment of an infectious disease caused by or associated with said pathogen. Preferred infectious pathogens are bacteria.

In some embodiments, the present invention provides protein constructs of the invention that comprise a targeting domain, e.g. a receptor, receptor domain, ligand, receptor ligand, or an antigen binding domain (e.g. constructs comprising antibodies, e.g. IgA or IgG antibodies or fragments thereof) that bind to the protein Her2 for use in the treatment of a Her2 positive cancer, e.g. a Her2 positive breast cancer. In some such embodiments, the antigen binding domain (the VL and VH domains) is, or is based on, the antibody Trastuzumab.

In other embodiments, the present invention provides protein constructs of the invention for use in transmucosal delivery of said protein construct, for example the therapeutic uses as described herein are carried out by transmucosal delivery of a protein construct of the invention by an appropriate administration route, e.g. by intranasal or pulmonary administration.

In another aspect, the present invention provides immunoconjugates of the invention for use in therapy, e.g. therapies as discussed elsewhere herein.

The present invention further provides the use of a protein construct, preferably a recombinant protein construct, of the invention in the manufacture of a medicament or composition for use in therapy or for use in the treatment or prevention of any of the above mentioned diseases or conditions.

The present invention further provides a method of treatment or prevention of any of the above mentioned diseases or conditions wherein said method comprises the step of administering to a subject in need thereof a therapeutically effective amount of a protein construct, preferably a recombinant protein construct, of the invention.

Alternative and preferred embodiments and features of the invention as described elsewhere herein, in particular with regard to therapeutic uses, apply equally to these methods of treatment and uses of the invention

Nucleic acid molecules or expression vectors of the invention can equally be used in the therapeutic methods or uses as described herein.

Treatment of diseases or conditions in accordance with the present invention (for example treatment of pre-existing disease) includes cure of said disease or conditions, or any reduction or alleviation of disease (e.g. reduction in disease severity) or symptoms of disease.

As will be clear from the disclosure elsewhere herein, the methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

The protein constructs and compositions and methods and uses of the present invention may be used in combination with other therapeutics or therapeutic agents.

The “combined” embodiments of the invention thus include, for example, where a protein construct of the invention is used in combination with an agent or therapeutic agent that is not operatively attached thereto. In other “combined” embodiments of the invention, a protein of the invention is an immunoconjugate wherein the protein construct of the invention is itself operatively associated or combined with the agent or therapeutic agent that is used in combination. The operative attachment includes all forms of direct and indirect attachment as described herein and known in the art.

The invention therefore provides compositions, pharmaceutical compositions, therapeutic kits and medicinal cocktails comprising, optionally in at least a first composition or container, a biologically effective amount of at least a first protein construct of the invention and a biologically effective amount of at least a second biological agent. The “at least a second biological agent” will often be a therapeutic agent, but it need not be.

Where therapeutic agents are included as the at least a second biological agent, such therapeutics will typically be those for use in connection with the treatment of one or more of the disorders defined above.

Thus, in certain embodiments “at least a second therapeutic agent” will be included in the therapeutic kit or cocktail. The term is chosen in reference to the protein construct of the invention being the first therapeutic agent.

In certain embodiments of the present invention, the second therapeutic agent may be a radiotherapeutic agent, chemotherapeutic agent, anti-angiogenic agent, apoptosis-inducing agent, anti-tubulin drug, anti-cellular or cytotoxic agent, steroid, cytokine antagonist, cytokine expression inhibitor, chemokine antagonist, chemokine expression inhibitor, ATPase inhibitor, anti-inflammatory agent, signalling pathway inhibitor, checkpoint inhibitor, anti-cancer agent, other antibodies or coagulant.

Speaking generally, the at least a second therapeutic agent may be administered to the subject substantially simultaneously with the protein construct of the invention; such as from a single pharmaceutical composition or from two pharmaceutical compositions administered closely together.

Alternatively, the at least a second therapeutic agent may be administered to the subject at a time sequential to the administration of the protein of the invention. “At a time sequential”, as used herein, means “staggered”, such that the at least a second therapeutic agent is administered to the subject at a time distinct to the administration of the protein construct of the invention. Generally, the two agents are administered at times effectively spaced apart to allow the two agents to exert their respective therapeutic effects, i.e., they are administered at “biologically effective time intervals”. The at least a second therapeutic agent may be administered to the subject at a biologically effective time prior to the protein of the invention, or at a biologically effective time subsequent to the administration of the protein of the invention.

The in vivo methods and uses as described herein (e.g. the therapeutic uses) are generally carried out in a mammal. Any mammal may be treated, for example humans and any livestock, domestic or laboratory animal. Specific examples include mice, rats, pigs, cats, dogs, sheep, rabbits, horses, cows and monkey (e.g. cynomolgus monkey). Preferably, however, the mammal is a human. Thus, the term “patient” or “subject” as used herein includes any such mammal. Thus, subjects or patients treated in accordance with the present invention will preferably be humans.

A therapeutically effective amount will be determined based on the clinical assessment and can be readily monitored.

The compositions and methods and uses of the present invention may be used in combination with other therapeutics and diagnostics.

The invention further includes kits comprising one or more of the protein constructs (or immunoconjugates) or compositions of the invention or one or more of the nucleic acid molecules encoding the protein constructs of the invention, or one or more expression vectors, e.g. recombinant expression vectors, comprising the nucleic acid molecules of the invention, or one or more host cells comprising the expression vectors, e.g. recombinant expression vectors, or nucleic acid molecules of the invention. Preferably said kits are for use in the methods and uses as described herein, e.g. in the therapeutic methods as described herein, or are for use in the in vitro assays or methods as described herein. Preferably said kits comprise instructions for use of the kit components. Preferably said kits are for treating or preventing diseases as described elsewhere herein, and optionally comprise instructions for use of the kit components to treat or prevent such diseases.

As used throughout the application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms. Methods comprising certain steps also include, where appropriate, methods consisting of these steps.

The term “increase” or “enhance” or “improve” (or equivalent terms) as described herein includes any measurable increase or elevation when compared with an appropriate control. Appropriate controls would readily be identified by a person skilled in the art and appropriate examples are described herein. Preferably the increase will be significant, for example clinically or statistically significant, for example with a probability value of <0.05 or ≤0.05, when compared to an appropriate control level or value.

The term “decrease” or “reduce” (or equivalent terms) as described herein includes any measurable decrease or reduction when compared with an appropriate control. Appropriate controls would readily be identified by a person skilled in the art and appropriate examples are described herein. Preferably the decrease will be significant, for example clinically or statistically significant, for example with a probability value of <0.05 or ≤0.05, when compared to an appropriate control level or value.

Methods of determining the statistical significance of differences in levels or values of a particular parameter are well known and documented in the art. For example herein a decrease or increase is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of <0.05 or ≤0.05.

### LIST OF SOME OF THE AMINO ACID SEQUENCES DISCLOSED HEREIN AND THEIR SEQUENCE IDENTIFIERS (SEQ ID NOs)

TABLE 1

| | | |
|---|---|---|
| SEQ | | |
| ID | | |
| NO: | Description | Sequence |

Heavy chain sequences

| | | |
|---|---|---|
| 1 | IgG1 heavy chain | ASTKGPSVFPLAPSSKSTSGGTAALGCLVKDYFPEPVTVSWN |
| | (human) | SGALTSGVHTFPAVLQSSGLYSLSSVVTVPSSSLGTQTYICN |
| | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPG |
| 2 | IgG2 heavy chain | ASTKGPSVFPLAPCSRSTSESTAALGCLVKDYFPEPVTVSWN |
| | (human) | SGALTSGVHTFPAVLQSSGLYSLSSVVTVPSSNFGTQTYTCN |
| | | VDHKPSNTKVDKTVERKCCVECPPCPAPPVAGPSVFLFPPKP |
| | | KDTLMISRTPEVTCVVVDVSHEDPEVQFNWYVDGVEVHNAKT |
| | | KPREEQFNSTFRVVSVLTVVHQDWLNGKEYKCKVSNKGLPAP |
| | | IEKTISKTKGQPREPQVYTLPPSREEMTKNQVSLTCLVKGFY |
| | | PSDIAVEWESNGQPENNYKTTPPMLDSDGSFFLYSKLTVDKS |
| | | RWQQGNVFSCSVMHEALHNHYTQKSLSLSPG |
| 3 | IgG3 heavy chain | ASTKGPSVFPLAPCSRSTSGGTAALGCLVKDYFPEPVTVSWN |
| | (human) | SGALTSGVHTFPAVLQSSGLYSLSSVVTVPSSSLGTQTYTCN |
| | | VNHKPSNTKVDKRVELKTPLGDTTHTCPRCPEPKSCDTPPPC |
| | | PRCPEPKSCDTPPPCPRCPEPKSCDTPPPCPRCPAPELLGGP |
| | | SVFLFPPKPKDTLMISRTPEVTCVVVDVSHEDPEVQFKWYVD |
| | | GVEVHNAKTKPREEQYNSTFRVVSVLTVLHQDWLNGKEYKCK |
| | | VSNKALPAPIEKTISKTKGQPREPQVYTLPPSREEMTKNQVS |
| | | LTCLVKGFYPSDIAVEWESSGQPENNYNTTPPMLDSDGSFFL |
| | | YSKLTVDKSRWQQGNIFSCSVMHEALHNRYTQKSLSLSPG |
| 4 | IgG4 heavy chain | ASTKGPSVFPLAPCSRSTSESTAALGCLVKDYFPEPVTVSWN |
| | (human) | SGALTSGVHTFPAVLQSSGLYSLSSVVTVPSSSLGTKTYTCN |
| | | VDHKPSNTKVDKRVESKYGPPCPPCSAPEFLGGPSVFLFPPK |
| | | PKDTLMISRTPEVTCVVVDVSQEDPEVQFNWYVDGVEVHNA |
| | | KTKPREEQFNSTYRVVSVLTVLHQDWLNGKEYKCKVSNKGL |
| | | PSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLG |
| 5 | IgA1 heavy chain | ASPTSPKVFPLSLCSTQPDGNVVIACLVQGFFPQEPLSVTWS |
| | (human), no | ESGQGVTARNFPPSQDASGDLYTTSSQLTLPATQCLAGKSV |
| | tailpiece | TCHVKHYTNPSQDVTVPCPVPSTPPTPSPSTPPTPSPSCCHP |
| | | RLSLHRPALEDLLLGSEANLTCTLTGLRDASGVTFTWTPSSG |
| | | KSAVQGPPERDLCGCYSVSSVLPGCAEPWNHGKTFTCTAAYP |
| | | ESKTPLTATLSKSGNTFRPEVHLLPPPSEELALNELVTLTCL |
| | | ARGFSPKDVLVRWLQGSQELPREKYLTWASRQEPSQGTTTF |
| | | AVTSILRVAAEDWKKGDTFSCMVGHEALPLAFTQKTIDRLA |
| 6 | IgA2 heavy chain | ASPTSPKVFPLSLDSTPQDGNVVVACLVQGFFPQEPLSVTWS |
| | (human), no | ESGQNVTARNFPPSQDASGDLYTTSSQLTLPATQCPDGKSV |
| | tailpiece | TCHVKHYTNPSQDVTVPCRVPPPPPCCHPRLSLHRPALEDLL |
| | | LGSEANLTCTLTGLRDASGATFTWTPSSGKSAVQGPPERDL |
| | | CGCYSVSSVLPGCAQPWNHGETFTCTAAHPELKTPLTANITK |
| | | SGNTFRPEVHLLPPPSEELALNELVTLTCLARGFSPKDVLVR |
| | | WLQGSQELPREKYLTWASRQEPSQGTTTFAVTSILRVAAED |
| | | WKKGDTFSCMVGHEALPLAFTQKTIDRLA |
| 7 | IgG1 heavy chain | ASTKGPSVFPLAPSSKSTSGGTAALGCLVKDYFPEPVTVSWNS |
| | (human)-REW | GALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHRDWLNGKEYKCKVSNKA |
| | | LPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVEHEALHWHYTQKSLSLSPG |
| 8 | IgG2 heavy chain | ASTKGPSVFPLAPCSRSTSESTAALGCLVKDYFPEPVTVSWN |
| | (human)-REW | SGALTSGVHTFPAVLQSSGLYSLSSVVTVPSSNFGTQTYTCN |
| | | VDHKPSNTKVDKTVERKCCVECPPCPAPPVAGPSVFLFPPKP |
| | | KDTLMISRTPEVTCVVVDVSHEDPEVQFNWYVDGVEVHNAK |
| | | TKPREEQFNSTFRVVSVLTVVHRDWLNGKEYKCKVSNKGLPA |
| | | PIEKTISKTKGQPREPQVYTLPPSREEMTKNQVSLTCLVKGF |
| | | YPSDIAVEWESNGQPENNYKTTPPMLDSDGSFFLYSKLTVDK |
| | | SRWQQGNVFSCSVEHEALHWHYTQKSLSLSPG |
| 9 | IgG3 heavy chain | ASTKGPSVFPLAPCSRSTSGGTAALGCLVKDYFPEPVTVSW |
| | (human)-REW | NSGALTSGVHTFPAVLQSSGLYSLSSVVTVPSSSLGTQTYTC |
| | | NVNHKPSNTKVDKRVELKTPLGDTTHTCPRCPEPKSCDTPPP |
| | | CPRCPEPKSCDTPPPCPRCPEPKSCDTPPPCPRCPAPELLG |
| | | GPSVFLFPPKPKDTLMISRTPEVTCVVVDVSHEDPEVQFKWY |
| | | VDGVEVHNAKTKPREEQYNSTFRVVSVLTVLHRDWLNGKEY |
| | | KCKVSNKALPAPIEKTISKTKGQPREPQVYTLPPSREEMTKN |
| | | QVSLTCLVKGFYPSDIAVEWESSGQPENNYNTTPPMLDSDG |
| | | SFFLYSKLTVDKSRWQQGNIFSCSVEHEALHWRYTQKSLSLS |
| | | PG |
| 10 | IgG3 heavy chain | ASTKGPSVFPLAPCSRSTSGGTAALGCLVKDYFPEPVTVSW |
| | (human)-REWH | NSGALTSGVHTFPAVLQSSGLYSLSSVVTVPSSSLGTQTYTC |
| | | NVNHKPSNTKVDKRVELKTPLGDTTHTCPRCPEPKSCDTPPP |
| | | CPRCPEPKSCDTPPPCPRCPEPKSCDTPPPCPRCPAPELLG |
| | | GPSVFLFPPKPKDTLMISRTPEVTCVVVDVSHEDPEVQFKWY |
| | | VDGVEVHNAKTKPREEQYNSTFRVVSVLTVLHRDWLNGKEY |
| | | KCKVSNKALPAPIEKTISKTKGQPREPQVYTLPPSREEMTKN |
| | | QVSLTCLVKGFYPSDIAVEWESSGQPENNYNTTPPMLDSDG |
| | | SFFLYSKLTVDKSRWQQGNIFSCSVEHEALHWHYTQKSLSLS |
| | | PG |
| 11 | IgG4 heavy chain | ASTKGPSVFPLAPCSRSTSESTAALGCLVKDYFPEPVTVSWN |
| | (human)-REW | SGALTSGVHTFPAVLQSSGLYSLSSVVTVPSSSLGTKTYTCN |
| | | VDHKPSNTKVDKRVESKYGPPCPPCSAPEFLGGPSVFLFPPK |
| | | PKDTLMISRTPEVTCVVVDVSQEDPEVQFNWYVDGVEVHNA |
| | | KTKPREEQFNSTYRVVSVLTVLHRDWLNGKEYKCKVSNKGL |
| | | PSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVEHEALHWHYTQKSLSLSLG |

Fc regions (CH2 and CH3)

| | | |
|---|---|---|
| 12 | IgG1 human Fc | APELLGGPSVFLFPPKPKDTLMISRTPEVTCVVVDVSHEDPE |
| | region | VKFNWYVDGVEVHNAKTKPREEQYNSTYRVVSVLTVLHQDWL |
| | | NGKEYKCKVSNKALPAPIEKTISKAKGQPREPQVYTLPPSRD |
| | | ELTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPG |
| 13 | IgG2 human Fc | APPVAGPSVFLFPPKPKDTLMISRTPEVTCVVVDVSHEDPEV |
| | region | QFNWYVDGVEVHNAKTKPREEQFNSTFRVVSVLTVVHQDWL |
| | | NGKEYKCKVSNKGLPAPIEKTISKTKGQPREPQVYTLPPSRE |
| | | EMTKNQVSLTCLVKGFYPSDIAVEWESNGQPENNYKTTPPM |
| | | LDSDGSFFLYSKLTVDKSRWQQGNVFSCSVMHEALHNHYTQ |
| | | KSLSLSPG |
| 14 | IgG3 human Fc | APELLGGPSVFLFPPKPKDTLMISRTPEVTCVVVDVSHEDPE |
| | region | VQFKWYVDGVEVHNAKTKPREEQYNSTFRVVSVLTVLHQDWL |
| | | NGKEYKCKVSNKALPAPIEKTISKTKGQPREPQVYTLPPSRE |
| | | EMTKNQVSLTCLVKGFYPSDIAVEWESSGQPENNYNTTPPML |
| | | DSDGSFFLYSKLTVDKSRWQQGNIFSCSVMHEALHNRYTQK |
| | | SLSLSPG |
| 15 | IgG4 human Fc | APEFLGGPSVFLFPPKPKDTLMISRTPEVTCVVVDVSQEDPE |
| | region | VQFNWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKTTP |
| | | PVLDSDGSFFLYSRLTVDKSRWQEGNVFSCSVMHEALHNHY |
| | | TQKSLSLSLG |
| 16 | IgA1 human Fc | HPRLSLHRPALEDLLLGSEANLTCTLTGLRDASGVTFTWTPS |
| | region | SGKSAVQGPPERDLCGCYSVSSVLPGCAEPWNHGKTFTCTAA |
| | | YPESKTPLTATLSKSGNTFRPEVHLLPPPSEELALNELVTLT |
| | | CLARGFSPKDVLVRWLQGSQELPREKYLTWASRQEPSQGTTT |
| | | FAVTSILRVAAEDWKKGDTFSCMVGHEALPLAFTQKTIDRLA |
| 17 | IgA2 human Fc | HPRLSLHRPALEDLLLGSEANLTCTLTGLRDASGATFTWTPS |
| | region | SGKSAVQGPPERDLCGCYSVSSVLPGCAQPWNHGETFTCTAA |
| | | HPELKTPLTANITKSGNTFRPEVHLLPPPSEELALNELVTLT |
| | | CLARGFSPKDVLVRWLQGSQELPREKYLTWASRQEPSQGTTT |
| | | FAVTSILRVAAEDWKKGDTFSCMVGHEALPLAFTQKTIDRLA |

Hinge regions

| | | |
|---|---|---|
| 18 | IgG1 hinge region | EPKSCDKTHTCPPCP |
| 19 | IgG2 hinge region | ERKCCVECPPCP |
| 20 | IgG3 hinge region | ELKTPLGDTTHTCPRCPEPKSCDTPPPCPRCPEPKSCDTPPP |
| | | CPRCPEPKSCDTPPPCPRCP |
| 21 | IgG4 hinge region | ESKYGPPCPPCS |
| 22 | IgA1 hinge region | PVPSTPPTPSPSTPPTPSPSCC |
| 23 | IgA2 hinge region | RVPPPPPCC |

CH1 regions

| | | |
|---|---|---|
| 24 | IgG1 human CH1 | ASTKGPSVFPLAPSSKSTSGGTAALGCLVKDYFPEPVTVSWN |
| | | SGALTSGVHTFPAVLQSSGLYSLSSVVTVPSSSLGTQTYICN |
| | | VNHKPSNTKVDKKV |
| 25 | IgG2 human CH1 | ASTKGPSVFPLAPCSRSTSESTAALGCLVKDYFPEPVTVSWN |
| | | SGALTSGVHTFPAVLQSSGLYSLSSVVTVPSSNFGTQTYTCN |
| | | VDHKPSNTKVDKTV |
| 26 | IgG3 human CH1 | ASTKGPSVFPLAPCSRSTSGGTAALGCLVKDYFPEPVTVSWN |
| | | SGALTSGVHTFPAVLQSSGLYSLSSVVTVPSSSLGTQTYTCN |
| | | VNHKPSNTKVDKRV |
| 27 | IgG4 human CH1 | ASTKGPSVFPLAPCSRSTSESTAALGCLVKDYFPEPVTVSWN |
| | | SGALTSGVHTFPAVLQSSGLYSLSSVVTVPSSSLGTKTYTCN |
| | | VDHKPSNTKVDKRV |
| 28 | IgA1 human CH1 | ASPTSPKVFPLSLCSTQPDGNVVIACLVQGFFPQEPLSVTWS |
| | | ESGQGVTARNFPPSQDASGDLYTTSSQLTLPATQCLAGKSVT |
| | | CHVKHYTNPSQDVTVPC |
| 29 | IgA2 human CH1 | ASPTSPKVFPLSLDSTPQDGNVVVACLVQGFFPQEPLSVTWS |
| | | ESGQNVTARNFPPSQDASGDLYTTSSQLTLPATQCPDGKSVT |
| | | CHVKHYTNPSQDVTVPC |
| 30 | IgA Tailpiece | PTHVNVSVVMAEVDGTCY |

Cysteines engaging in disulphide bonds are shown in bold underline
REW = Q311R, M428E and N434W substitutions, shown in bold
REWH = Q311R, M428E, N434W and R435H substitutions, shown in bold

Exemplary sequences of some IgA Fc-IgG Fc fusions for use in the constructs of the invention are shown below. These all take the format IgA CH1, IgA hinge, IgA CH2, IgA CH3, IgG hinge, IgG CH2, IgG CH3.

>IgA1-IgG1-WT
(SEQ ID NO: 31)
ASPTSPKVFPLSLCSTQPDGNVVIACLVQGFFPQEPLSVTWSESG
QGVTARNFPPSQDASGDLYTTSSQLTLPATQCLAGKSVTCHVKHY
TNPSQDVTVPCPVPSTPPTPSPSTPPTPSPSCCHPRLSLHRPALE
DLLLGSEANLTCTLTGLRDASGVTFTWTPSSGKSAVQGPPERDLC
GCYSVSSVLPGCAEPWNHGKTFTCTAAYPESKTPLTATLSKSGNT
FRPEVHLLPPPSEELALNELVTLTCLARGFSPKDVLVRWLQGSQE
LPREKYLTWASRQEPSQGTTTFAVTSILRVAAEDWKKGDTFSCMV
GHEALPLAFTQKTIDRLAEPKSCDKTHTCPPCPAPELLGGPSVFL
FPPKPKDTLMISRTPEVTCVVVDVSHEDPEVKFNWYVDGVEVHNA
KTKPREEQYNSTYRVVSVLTVLHQDWLNGKEYKCKVSNKALPAPI
EKTISKAKGQPREPQVYTLPPSREEMTKNQVSLTCLVKGFYPSDI
AVEWESNGQPENNYKTTPPVLDSDGSFFLYSKLTVDKSRWQQGNV
FSCSVMHEALHNHYTQKSLSLSPGK-
>IgA1-IgG1-REW
(SEQ ID NO: 32)
ASPTSPKVFPLSLCSTQPDGNVVIACLVQGFFPQEPLSVTWSESG
QGVTARNFPPSQDASGDLYTTSSQLTLPATQCLAGKSVTCHVKHY
TNPSQDVTVPCPVPSTPPTPSPSTPPTPSPSCCHPRLSLHRPALE
DLLLGSEANLTCTLTGLRDASGVTFTWTPSSGKSAVQGPPERDLC
GCYSVSSVLPGCAEPWNHGKTFTCTAAYPESKTPLTATLSKSGNT
FRPEVHLLPPPSEELALNELVTLTCLARGFSPKDVLVRWLQGSQE
LPREKYLTWASRQEPSQGTTTFAVTSILRVAAEDWKKGDTFSCMV
GHEALPLAFTQKTIDRLAEPKSCDKTHTCPPCPAPELLGGPSVFL
FPPKPKDTLMISRTPEVTCVVVDVSHEDPEVKFNWYVDGVEVHNA
KTKPREEQYNSTYRVVSVLTVLHRDWLNGKEYKCKVSNKALPAPI
EKTISKAKGQPREPQVYTLPPSREEMTKNQVSLTCLVKGFYPSDI
AVEWESNGQPENNYKTTPPVLDSDGSFFLYSKLTVDKSRWQQGNV
FSCSVEHEALHWHYTQKSLSLSPGK-
>IgA2-IgG1-WT
(SEQ ID NO: 33)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK-
>IgA2-IgG1-REW
(SEQ ID NO: 34)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHRDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVEHEALHWH
YTQKSLSLSPGK-
>IgA2.0-IgG1-REW
(SEQ ID NO: 35)
ASPTSPKVFPLSLDSTPQDGNVVVACLVQGFFPQEPLSVTWSESG
QGVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGSA
QPWNHGETFTCTAAHPELKTPLTATLSKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHRDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVEHEALHWH
YTQKSLSLSPGK-
>IgA2-IgG2-WT
(SEQ ID NO: 36)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAERKCCVECPPCPAPPVAGPSVFLFPPKPKDTLMISRTPEV
TCVVVDVSHEDPEVQFNWYVDGVEVHNAKTKPREEQFNSTFRVVS
VLTVVHQDWLNGKEYKCKVSNKGLPAPIEKTISKTKGQPREPQVY
TLPPSREEMTKNQVSLTCLVKGFYPSDIAVEWESNGQPENNYKTT
PPMLDSDGSFFLYSKLTVDKSRWQQGNVFSCSVMHEALHNHYTQK
SLSLSPGK-
>IgA2-IgG2-REW
(SEQ ID NO: 37)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAERKCCVECPPCPAPPVAGPSVFLFPPKPKDTLMISRTPEV
TCVVVDVSHEDPEVQFNWYVDGVEVHNAKTKPREEQFNSTFRVVS
VLTVVHRDWLNGKEYKCKVSNKGLPAPIEKTISKTKGQPREPQVY
TLPPSREEMTKNQVSLTCLVKGFYPSDIAVEWESNGQPENNYKTT
PPMLDSDGSFFLYSKLTVDKSRWQQGNVFSCSVEHEALHWHYTQK
SLSLSPGK-
>IgA2-IgG4-WT
(SEQ ID NO: 38)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAESKYGPPCPPCSAPEFLGGPSVFLFPPKPKDTLMISRTPE
VTCVVVDVSQEDPEVQFNWYVDGVEVHNAKTKPREEQFNSTYRVV
SVLTVLHQDWLNGKEYKCKVSNKGLPSSIEKTISKAKGQPREPQV
YTLPPSQEEMTKNQVSLTCLVKGFYPSDIAVEWESNGQPENNYKT
TPPVLDSDGSFFLYSRLTVDKSRWQEGNVFSCSVMHEALHNHYTQ
KSLSLSLGK-
>IgA2-IgG4-REW
(SEQ ID NO: 39)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAESKYGPPCPPCSAPEFLGGPSVFLFPPKPKDTLMISRTPE
VTCVVVDVSQEDPEVQFNWYVDGVEVHNAKTKPREEQFNSTYRVV
SVLTVLHRDWLNGKEYKCKVSNKGLPSSIEKTISKAKGQPREPQV
YTLPPSQEEMTKNQVSLTCLVKGFYPSDIAVEWESNGQPENNYKT
TPPVLDSDGSFFLYSRLTVDKSRWQEGNVFSCSVEHEALHWHYTQ
KSLSLSLGK-
>IgA2-IgG3-WT
(SEQ ID NO: 40)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAELKTPLGDTTHTCPRCPEPKSCDTPPPCPRCPEPKSCDTP
PPCPRCPEPKSCDTPPPCPRCPAPELLGGPSVFLFPPKPKDTLMI
SRTPEVTCVVVDVSHEDPEVQFKWYVDGVEVHNAKTKPREEQYNS
TFRVVSVLTVLHQDWLNGKEYKCKVSNKALPAPIEKTISKTKGQP
REPQVYTLPPSREEMTKNQVSLTCLVKGFYPSDIAVEWESSGQPE
NNYNTTPPMLDSDGSFFLYSKLTVDKSRWQQGNIFSCSVMHEALH
NRYTQKSLSLSPGK-
>IgA2-IgG3-REWH
(SEQ ID NO: 41)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCRVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRLAELKTPLGDTTHTCPRCPEPKSCDTPPPCPRCPEPKSCDTP
PPCPRCPEPKSCDTPPPCPRCPAPELLGGPSVFLFPPKPKDTLMI
SRTPEVTCVVVDVSHEDPEVQFKWYVDGVEVHNAKTKPREEQYNS
TFRVVSVLTVLHRDWLNGKEYKCKVSNKALPAPIEKTISKTKGQP
REPQVYTLPPSREEMTKNQVSLTCLVKGFYPSDIAVEWESSGQPE
NNYNTTPPMLDSDGSFFLYSKLTVDKSRWQQGNIFSCSVEHEALH
WHYTQKSLSLSPGK-

Exemplary sequences of some IgA Fc-IgG Fc fusions for use in the extended constructs of the invention are shown below. These all take the format IgG CH1, IgG hinge, IgA CH2, IgA CH3, IgG hinge, IgG CH1, IgG hinge, IgG CH2, IgG CH3.

>IgG1Fab2-IgA2 Fc-H1-CH1-H1-IgG1Fc-WT
(SEQ ID NO: 42)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPHPRLSLHRPALEDLLLGSEANL
TCTLTGLRDASGATFTWTPSSGKSAVQGPPERDLCGCYSVSSVLP
GCAQPWNHGETFTCTAAHPELKTPLTANITKSGNTFRPEVHLLPP
PSEELALNELVTLTCLARGFSPKDVLVRWLQGSQELPREKYLTWA
SRQEPSQGTTTFAVTSILRVAAEDWKKGDTFSCMVGHEALPLAFT
QKTIDRLAEPKSCDKTHTCPPCPASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPELLGGPSVFLFPPKPKDTLMISRTPEVTCVVVDVSHEDPEVK
FNWYVDGVEVHNAKTKPREEQYNSTYRVVSVLTVLHQDWLNGKEY
KCKVSNKALPAPIEKTISKAKGQPREPQVYTLPPSREEMTKNQVS
LTCLVKGFYPSDIAVEWESNGQPENNYKTTPPVLDSDGSFFLYSK
LTVDKSRWQQGNVFSCSVMHEALHNHYTQKSLSLSPGK
> IgG1Fab2-IgA2 Fc-H1-CH1-H1-IgG1Fc-REW
(SEQ ID NO: 43)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPHPRLSLHRPALEDLLLGSEANL
TCTLTGLRDASGATFTWTPSSGKSAVQGPPERDLCGCYSVSSVLP
GCAQPWNHGETFTCTAAHPELKTPLTANITKSGNTFRPEVHLLPP
PSEELALNELVTLTCLARGFSPKDVLVRWLQGSQELPREKYLTWA
SRQEPSQGTTTFAVTSILRVAAEDWKKGDTFSCMVGHEALPLAFT
QKTIDRLAEPKSCDKTHTCPPCPASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPELLGGPSVFLFPPKPKDTLMISRTPEVTCVVVDVSHEDPEVK
FNWYVDGVEVHNAKTKPREEQYNSTYRVVSVLTVLHRDWLNGKEY
KCKVSNKALPAPIEKTISKAKGQPREPQVYTLPPSREEMTKNQVS
LTCLVKGFYPSDIAVEWESNGQPENNYKTTPPVLDSDGSFFLYSK
LTVDKSRWQQGNVFSCSVEHEALHWHYTQKSLSLSPGK

The invention will now be further described in the following non-limiting Example with reference to the following figures.

FIG. 1: Design of anti-Her2 antibody formats. Four antibody variants were produced based on IgG1 and IgA2. a) IgA2 without the tailpiece harbors the Fc binding site for the FcαRI, b) IgG1 with the overlapping binding sites for the FcγRs and C1q in the lower hinge and CH2 domain and FcRn at the CH2-CH3 elbow region. An Fc-engineered IgG1 variant was included with the PGLALA substitutions that abolish binding to low affinity FcγRs and C1q but not FcRn. c) The tandem IgG1-IgA2 variant consists of full-length IgG1 fused to the IgA2-Fc, without the tailpiece, including its hinge region. d) The tandem IgA2-IgG1 variant consists of full-length IgA2, without the tailpiece, fused to IgG1-Fc including its hinge region. The figure was made in BioRender.

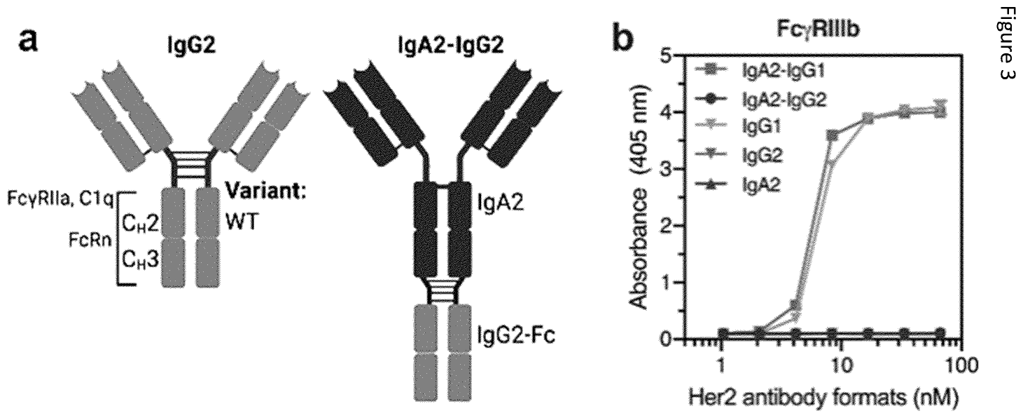

FIG. 2: pH-dependent human FcRn binding and extended plasma half-life. a) ELISA set-up performed at pH 5.5 and pH 7.4, where wells were coated with titrated amounts of the antibody variants, followed by adding biotinylated human FcRn pre-incubated with ALP-conjugated streptavidin (STV). Only one tandem orientation is illustrated for simplicity. The figure was made in BioRender. b) ELISA results showing binding of human FcRn to titrated amounts of the antibody variants at pH 5.5 and pH 7.4. Shown as mean±SD of duplicates from a representative experiment. c) HERA results showing the amounts of antibody variants detected in the medium by ELISA after the recycling step. Each of the antibodies was added to the HMEC-1 cells for 3 hours, followed by extensive washing and additional 3 hours of incubation before the medium was collected. Shown as mean±SD of triplicates from a representative experiment. d) Plasma half-life study in human FcRn transgenic (Tg) mice, where the antibody formats were given intravenously followed by blood samples collected from day 1 until day 23. The figure was made in BioRender. e) Plasma half-life (%) of the antibody formats (n=5), shown as mean±SD. ns>0.05, ***=p<0.001, by two-tailed unpaired T-test.

FIG. 3: The IgA2-IgG2 format and binding of FcγRIIIb. a) IgG2 harbors distinct binding sites for the FcγRs, C1q and FcRn. IgA2-IgG2 consists of full-length IgA2, without the tailpiece, fused to the IgG2-Fc including its hinge region. b) ELISA results showing binding of FcγRIIIb to titrated amounts of the antibody variants directly coated in wells, performed at pH 7.4. Shown as mean±SD of duplicates from a representative experiment.

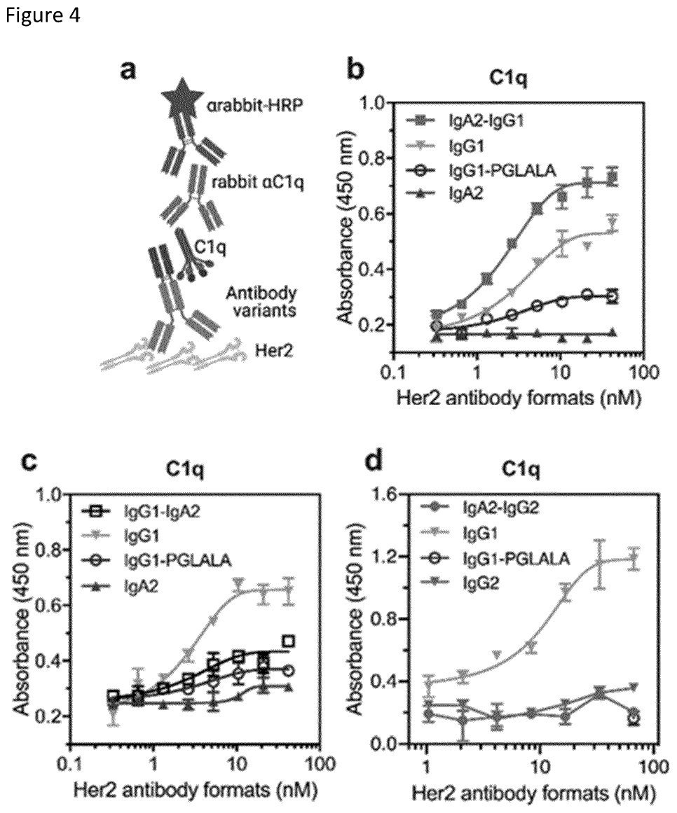

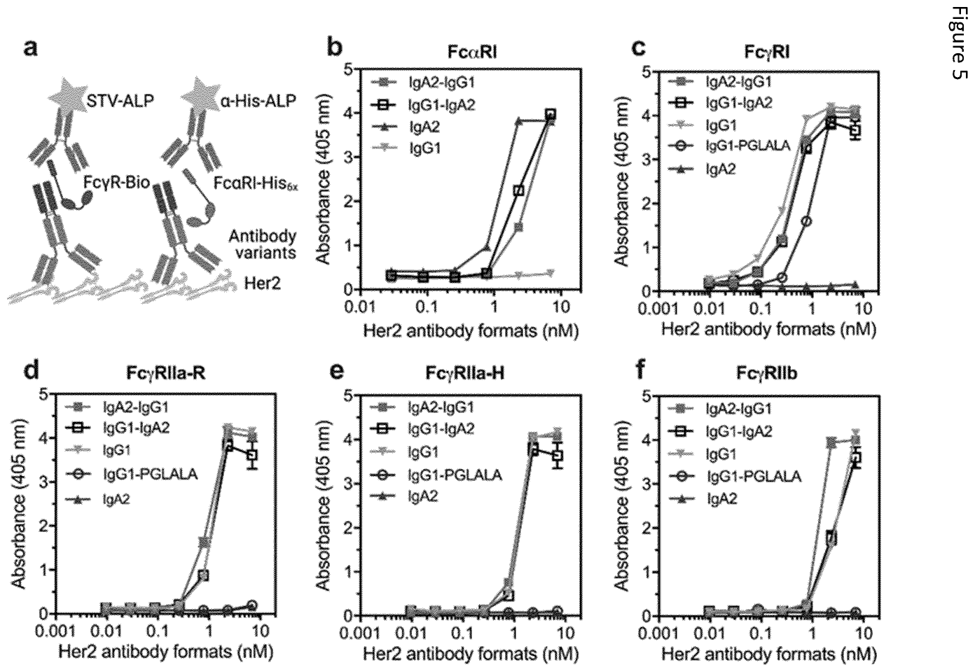

FIG. 4: Enhanced on-target C1q binding of IgA2-IgG1. a) ELISA set-up used to measure binding of C1q to Her2 captured antibody variants. Bound C1q was detected by a primary rabbit anti-C1q antibody and a secondary anti-rabbit HRP-conjugated antibody. b-d) ELISA results showing C1q binding to titrated amounts of the anti-Her2 IgG-based antibody variants. Shown as mean±SD of duplicates from a representative experiment.

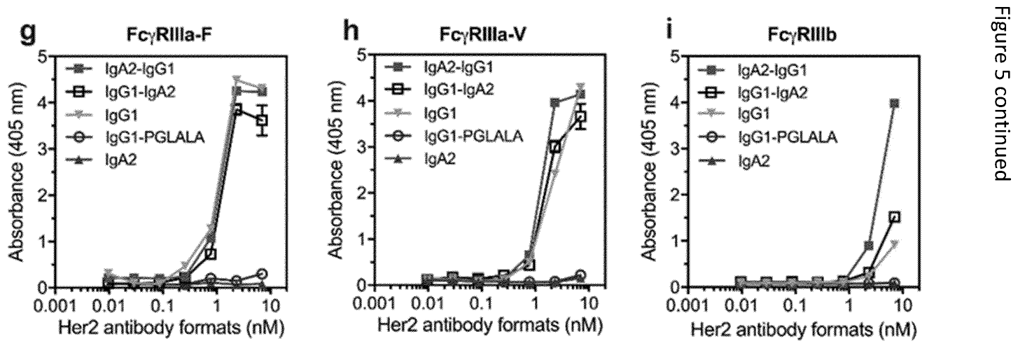

FIG. 5: Binding of Her2 antibody variants to the FcγRs and FcαRI. a) ELISA set-ups performed with recombinant Her2 coated in wells, followed by adding titrated amounts of the antibody formats. Either Hisex-tagged human FcαRI or site-specific biotinylated human FcγRs were added and detected with anti-His₆ₓ-ALP or ALP-conjugated streptavidin (STV), respectively. Only one tandem orientation is shown for simplicity. The figure was made in BioRender. ELISA results for b) FcαRI, c) FcγRI, d) FcγRIIa-R131, e) FcγRIIa-H131, f) FcγRIIb, g) FcγRIIIa-F158, h) FcγRIIIa-V158 and i) FcγRIIIb at pH 7.4. Shown as mean±SD of duplicates from representative experiments.

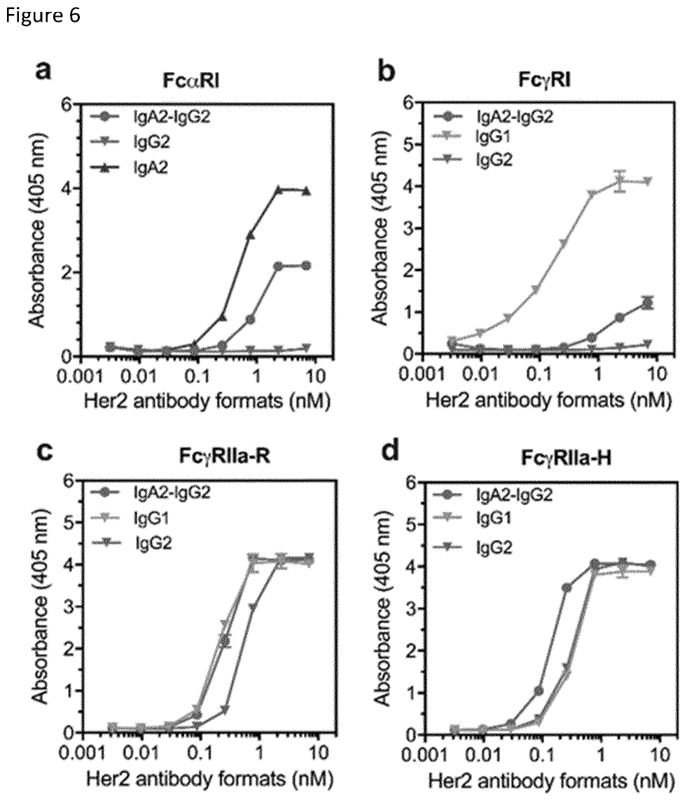

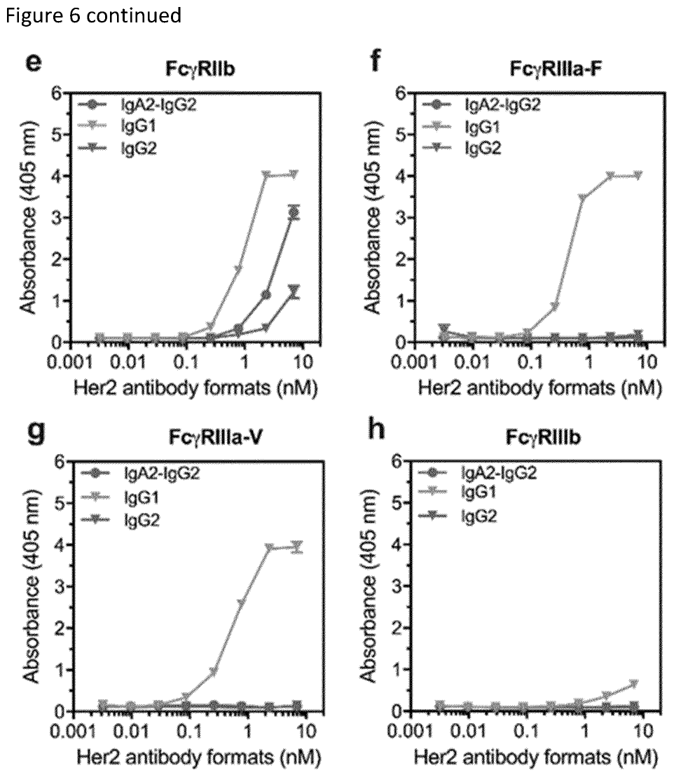

FIG. 6: Fc receptor binding capacity of anti-Her2 IgG2-based antibody formats. ELISA results showing binding of the receptors to titrated amounts of the antibody variants. a) His₆ₓ-tagged human FcαRI, site-specific biotinylated b) FcγRI, c) FcγRIIa-R131, d) FcγRIIa-H131, e) FcγRIIb, f) FcγRIIIa-F158, g) FcγRIIIa-V158 and h) FcγRIIIb at pH 7.4. Shown as mean±SD of duplicates from representative experiments.

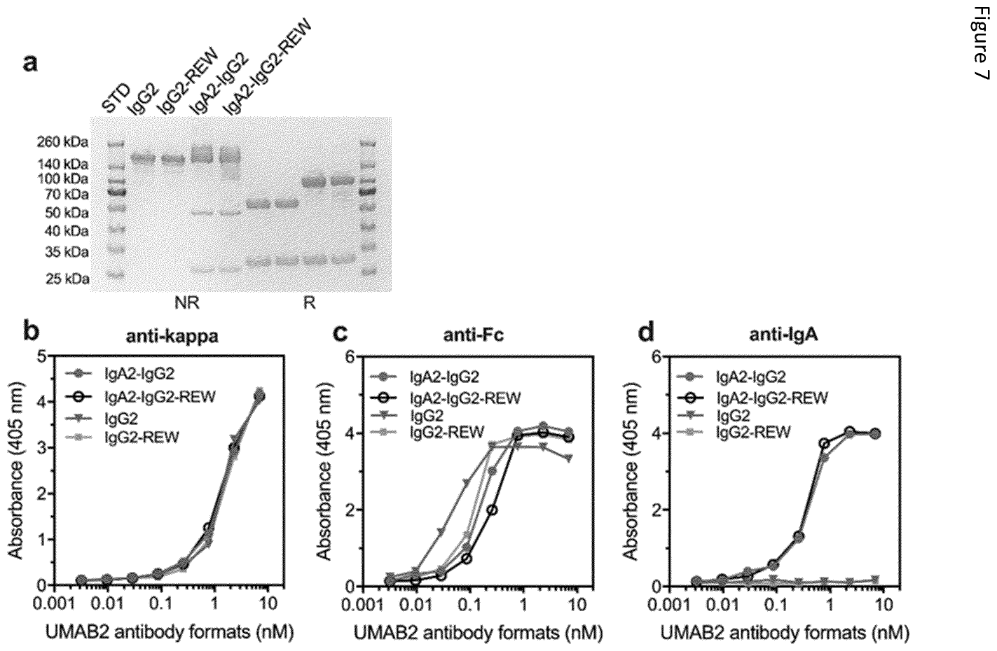

FIG. 7: Protein integrity and binding properties of UMAB2 IgG2-based antibody formats. a) Non-reducing (NR) and reducing (R) SDS-PAGE analysis of affinity and size-exclusion purified antibodies. b-d) Binding of the antibodies to anti-kappa, anti-Fc and anti-IgA antibodies in ELISA. Shown as mean±SD of duplicates from representative experiments.

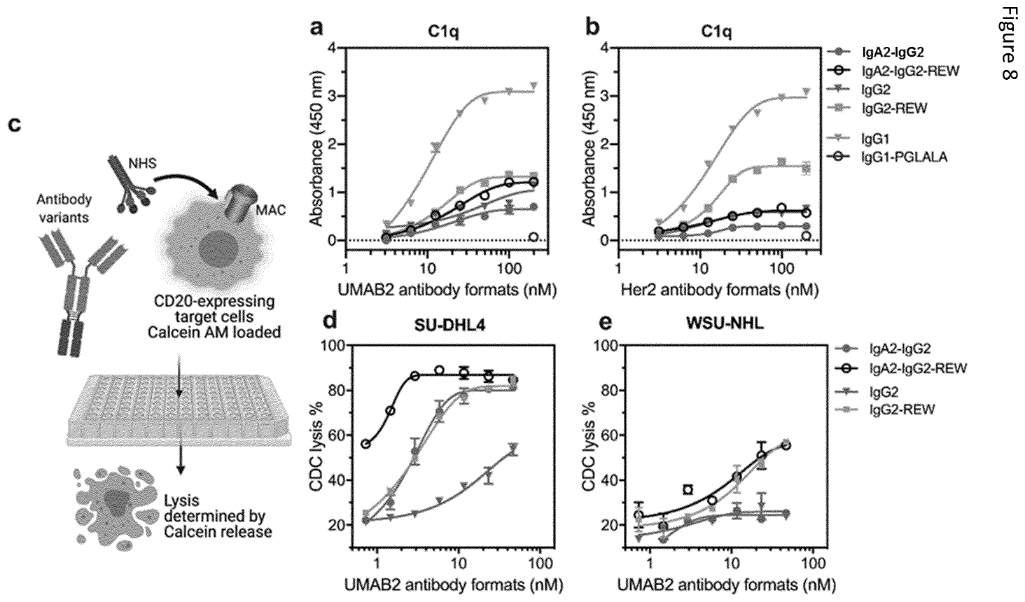

FIG. 8: C1q binding and CDC activity in a Calcein AM-based assay. ELISA results showing C1q binding to titrated amounts of a) anti-CD20 and b) anti-Her2 antibody variants. c) Schematic illustration of the CDC assay, where CD20-expressing target cells are loaded with Calcein AM and mixed with NHS and the antibody variants, and lysis of cells determined by Calcein release. Only one tandem orientation is illustrated for simplicity. The figure was made in BioRender. d-e) Specific lysis (%) in the presence of antibody variants and NHS of the Calcein loaded CD20-expressing cell lines, with WSU-NHL and SU-DHL4 as target cells. Shown as mean±SD of duplicates from representative experiments.

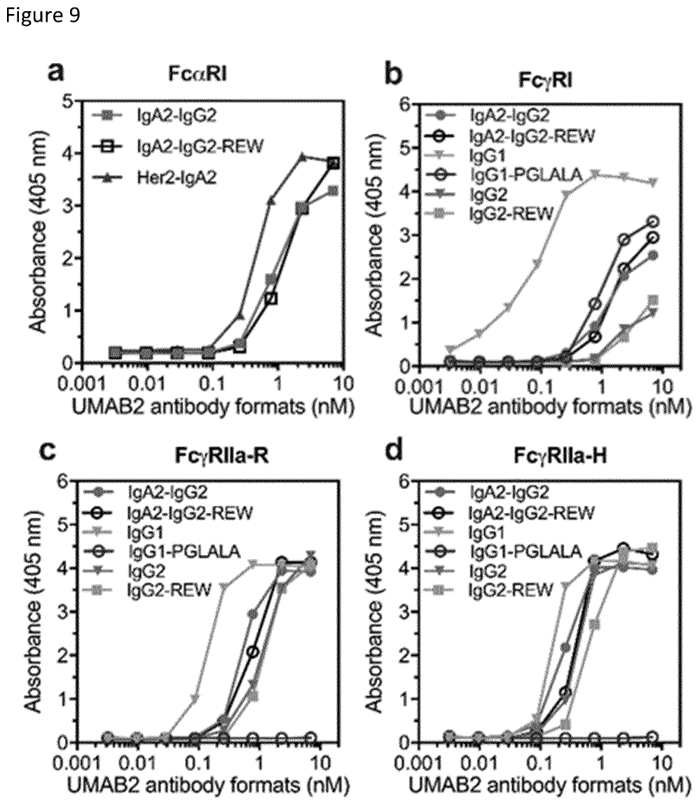

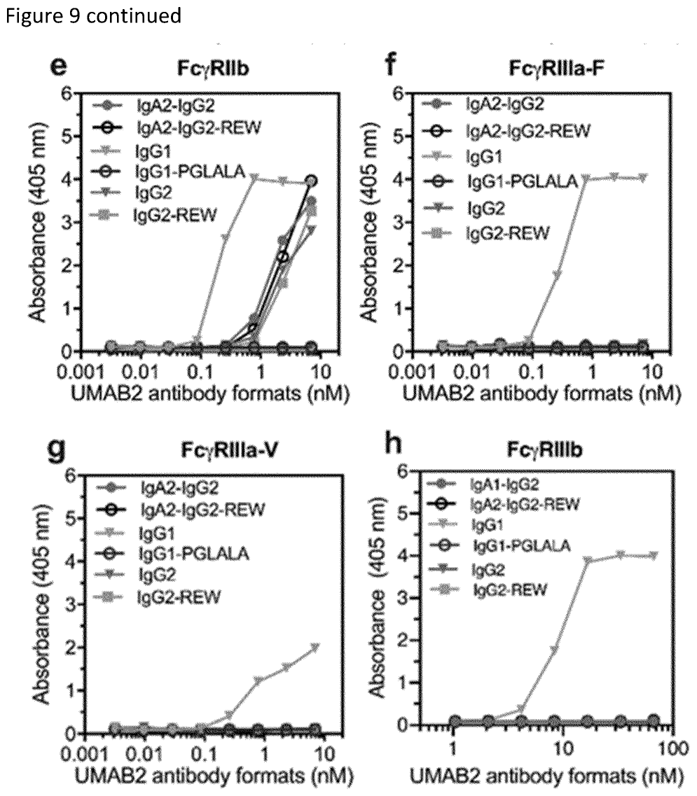

FIG. 9: Fc receptor binding capacity of anti-CD20 antibody formats. ELISA results showing binding of the receptors to titrated amounts of the antibody variants. a) Hisex-tagged human FcαRI, site-specific biotinylated b) FcγRI, c) FcγRIIa-R131, d) FcγRIIa-H131, e) FcγRIIb, f) FcγRIIIa-F158, g) FcγRIIIa-V158 and h) FcγRIIIb at pH 7.4. Shown as mean±SD of duplicates from representative experiments.

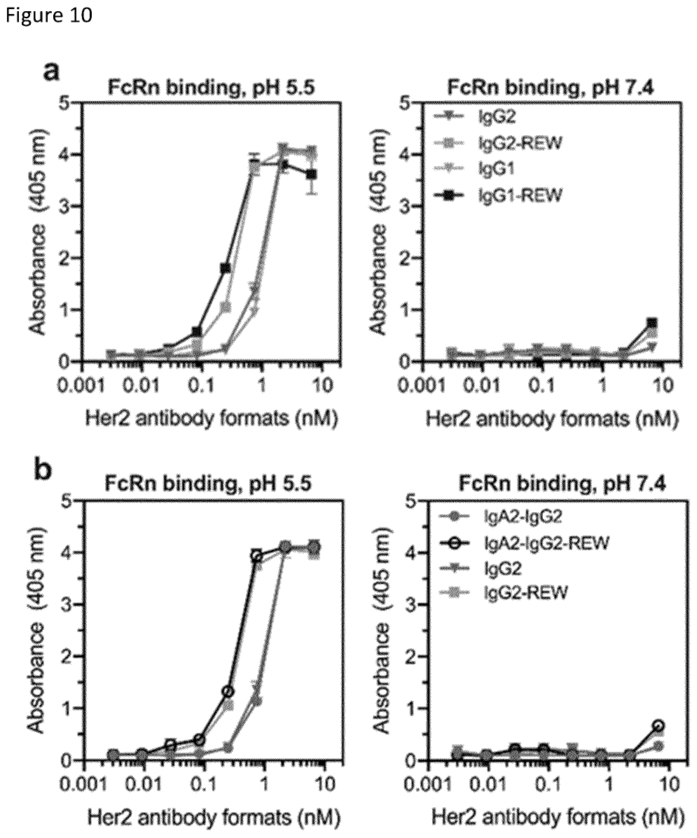

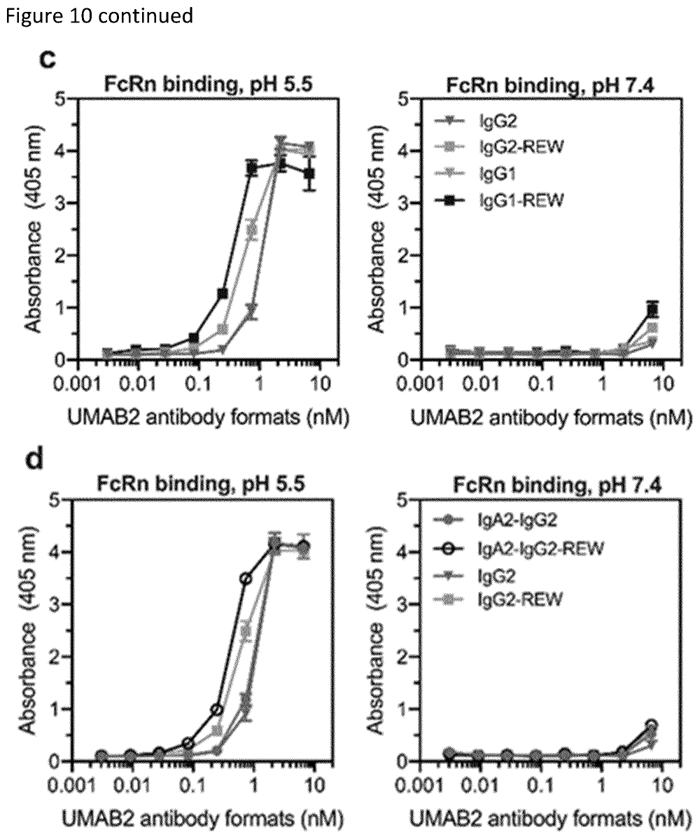

FIG. 10: pH-dependent human FcRn binding of antibody variants. a-b) ELISA results showing binding of human FcRn to titrated amounts of anti-Her2 antibody variants at pH 5.5 and pH 7.4. c-d) ELISA results showing binding of human FcRn to titrated amounts of anti-CD20 antibody variants at pH 5.5 and pH 7.4. Shown as mean±SD of duplicates from representative experiments.

FIG. 11: CDC activity against Raji target cells in a Calecin-AM based assay. a-b) Specific lysis (%) of CD20 expressing Raji target cells in presence of UMAB2 antibody variants and normal human serum (NHS). Shown as mean±s.e.m of duplicates from three independent experiments.

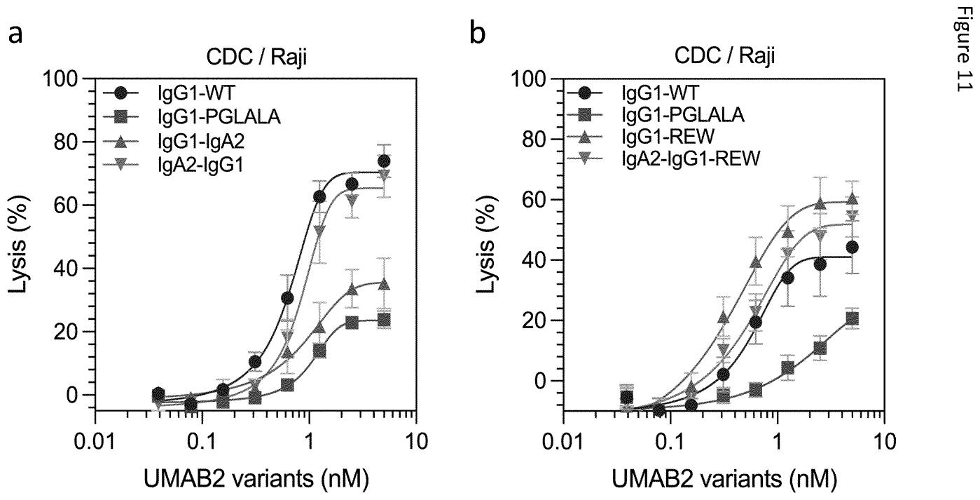

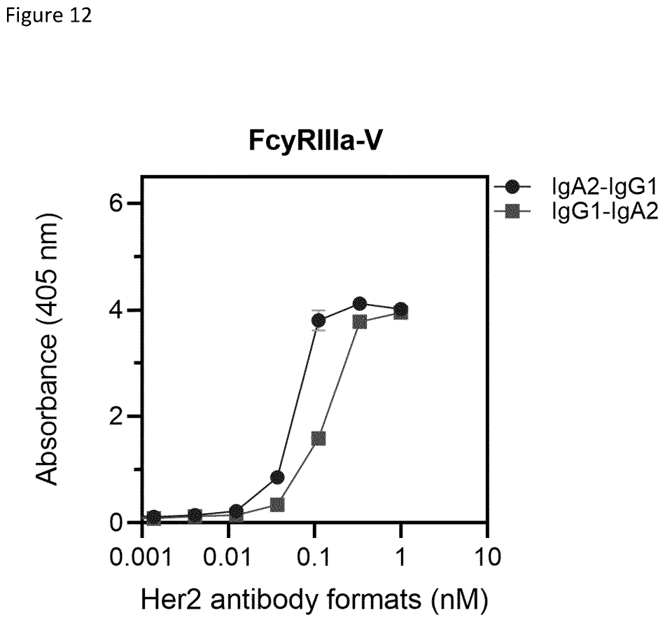

FIG. 12: FcγRIIIa binding capacity of anti-Her2 antibody formats. ELISA results showing binding of FcγRIIIa (V158 allotype) to Her2 specific IgA2-IgG1 and IgG1-IgA2 tandem formats. Shown as mean±S.D of duplicates from a representative experiment.

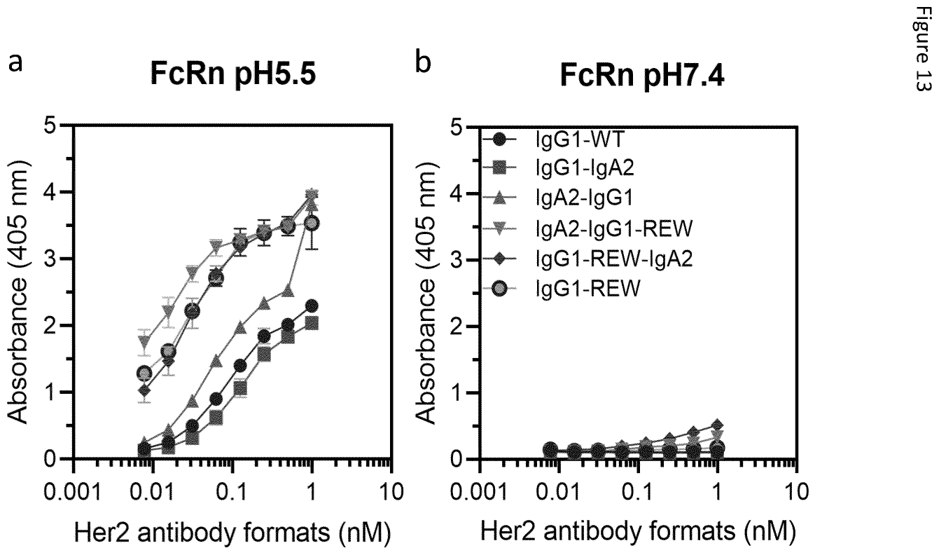

FIG. 13: pH-dependent FcRn binding of anti-Her2 antibody variants. a-b) ELISA results showing binding of human FcRn to titrated amounts of anti-Her2 antibody variants at pH 5.5 and pH 7.4. Shown as mean±SD of duplicates from a representative experiment.

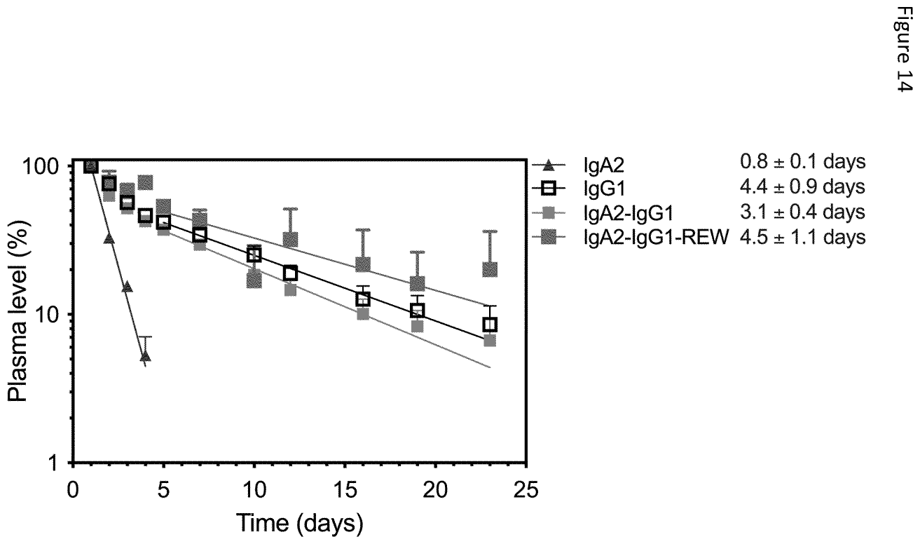

FIG. 14: Plasma half-life of antibody formats in human FcRn expressing mice in presence of competition. Plasma half-life of antibody variants in human FcRn transgenic mice pre-loaded with 500 mg/kg of IVIg 2 days prior to administration of test antibodies. The antibody variants were administered intravenously on day 0 followed by blood sample collection from day 1 until day 23. Shown as mean±s.e.m (n=5).

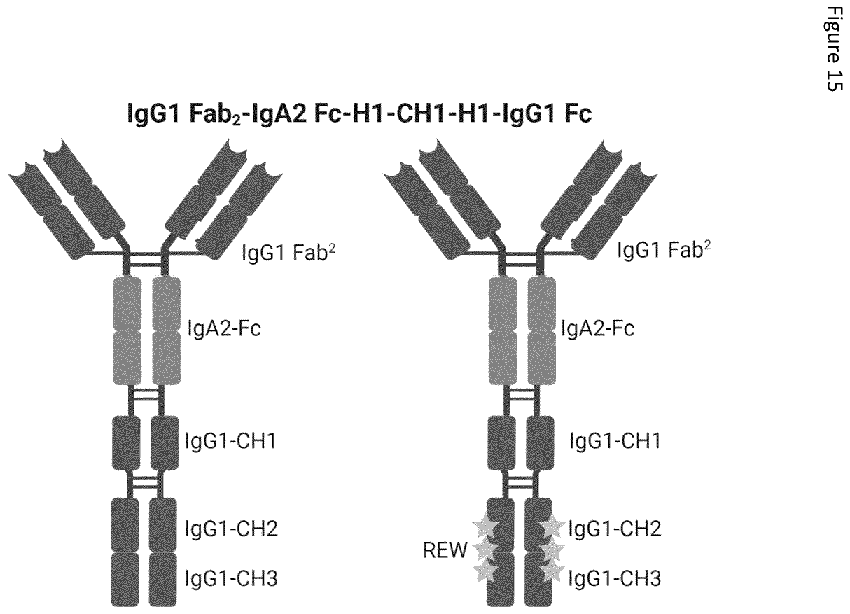

FIG. 15: Design of novel IgG1/IgA2 antibody formats. Two additional antibody variants were designed based on IgG1 and IgA2 specific for CD20 (UMAB2). The formats contain a IgG1 Fab₂ fused to an IgA2 Fc (Cα2 and Cα3) followed by an IgG1 hinge, an IgG1 C_{H}1, a third IgG1 hinge and a C-terminal IgG1 Fc (C_{H}2 and C_{H}3). The C-terminal IgG1 Fc is either WT or modified with the REW technology amino acid substitutions.

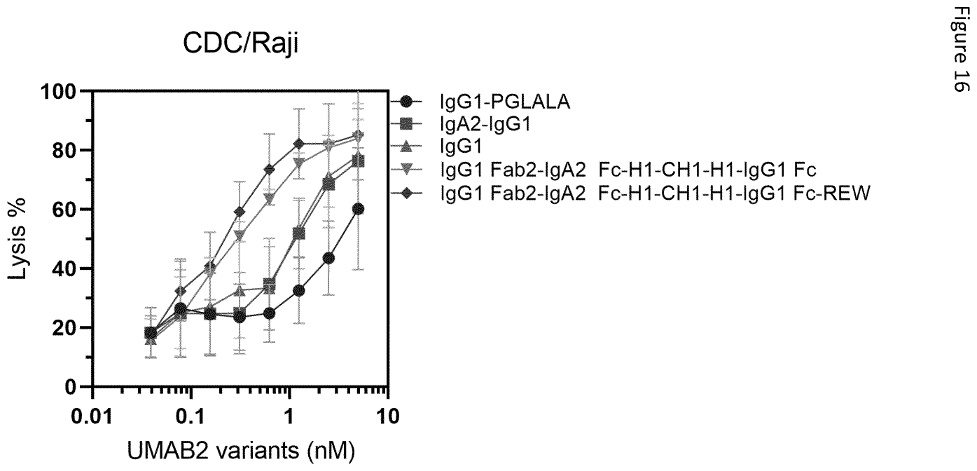

FIG. 16: CDC activity of novel IgG1/IgA2 tandem formats against Raji target cells in a Calecin-AM based assay. Specific lysis (%) of CD20 expressing Raji target cells in presence of novel UMAB2 IgG1/IgA2 tandem formats and NHS. Shown as mean±s.e.m of duplicates from a representative experiment.

### EXAMPLE 1: PREPARATION OF TANDEM IGA FC-IGG FC FUSION PROTEINS

Here, we report on a panel of antibody designs that combine structural elements from IgA and IgG, specifically an IgA Fc and an IgG Fc, allowing for efficient engagement of both the FcγRs and FcαRI. At the same time, we identified an IgA-IgG tandem format that was efficiently rescued from intracellular degradation by FcRn, which translated into long plasma half-life in human FcRn transgenic mice. This format combines full-length IgA2 with the IgG1-Fc and is shown to give rise to good induction of CDC-induced killing of CD20-expressing Raji cells and good pH dependent binding to FcRn. In addition, when anti-CD20 IgA2-IgG2 was combined with an Fc-engineering strategy for enhanced on-target hexamer formation, the tandem format gave rise to potent induction of CDC-induced killing of CD20-expressing cancer B cell lines.

### Materials and Methods

### Antibody Production and Purification

DNA segments encoding the variable sequences from anti-HER2 trastuzumab or anti-CD20 UMAB2 (Meyer, S. et al. British journal of haematology 180, 808-820 (2018)) were sub-cloned in frame with the IgA2-HC and kappa-LC encoding sequences in the previously described vectors pEE14.4-kappaLC, pEE14.4-IgA1 and pEE14.4-IgA2 (m1). For the tandem variants, cDNA encoding the full-length IgG1-HC was sub-cloned in frame with the sequence encoding the hinge and IgA2-C_{H}2-C_{H}3 domains. The DNA segment encoding the tailpiece of IgA2 was deleted. For the IgA2-IgG1/2-Fc tandem variants, cDNA encoding IgG1-Fc, IgG2-Fc and IgG2-Fc-REW with their corresponding hinge regions included were sub-cloned in frame with the DNA corresponding to the IgA2-HC. For IgG1 Fab₂-IgA2 Fc-H1-CH1-H1-IgG1 Fc extended tandem variants as used in Example 2, cDNA encoding the entire constant region was synthesized and then subcloned in frame with trastuzumab or UMAB2 variable regions.

The adherent HEK293E and Expi293 suspension cell lines were transiently co-transfected with the HC and LC encoding vectors using Lipofectamine2000 or Expifectamine (ThermoFisher), respectively. Harvested supernatant was applied on a CaptureSelect IgG-C_{H}1 pre-packed column (ThermoFisher) or a CaptureSelect IgA affinity matrix (Thermofisher) packed in a column (Atoll), as described by the manufacturer. The eluted purified proteins were buffer-exchanged to phosphate buffered saline (PBS) (Merck), concentrated using Amicon Ultra-15 ml 100K spin columns (Millipore), and then purified by size-exclusion chromatography using a Superdex 200 increase 10/300GL column (GE Healthcare) coupled to an ÄKTA Avant instrument (Cytiva Lifesciences). Eluted monomeric proteins were concentrated using Amicon Ultra-5 ml 100K columns (Millipore) and analyzed using SDS-PAGE (ThermoFisher).

### ELISA

ELISAs were performed by coating antigen or antibody variants, diluted in PBS (100 μl) into EIA/RIA 96-well plates (CorningCostar), incubated overnight (ON) at 4° C. The next day, plates were blocked with 250 μl PBS (Merck) containing 4% skimmed milk(S) (ITW reagents) for 1 hour at room temperature (RT). Between all subsequent layers, the plates were washed 4 times using 250 μl PBS containing 0.05% Tween 20 (T) (Merck). All samples were added in a total volume of 100 μl diluted in PBS/S/T, if not stated otherwise, and incubated for 1-2 hours at RT.

To study antigen binding, recombinant human Her2 (1.0 μg/ml; Sino Biological) was coated in wells, followed by adding titrated amounts of the antibodies (7.0-0.0032 nM) the following day. ALP-conjugated anti-human kappa LC (Southern Biotech), anti-human IgG-Fc (Merck) or anti-IgA-Fc antibody (Merck) was used as a second layer, where binding was visualized by adding the ALP substrate (Merck) solved in diethanolamine buffer (pH 9.8). Absorbance was measured at 405 nm with a Sunrise spectrophotometer (TECAN). The same set-up was used to measure binding of Hisex-tagged human FcαRI (2 μg/ml; Sino Biological) which was detected with an ALP-conjugated anti-Hisex-tag antibody (Abcam) (1:5000). In addition, binding to site-specific biotinylated FcγRI, FcγRIIa-H131, FcγRIIa-R131, FcγRIIb, FcγRIIIa-V156, FcγRIIIa-F156 and FcγRIIIb (0.25 μg/ml; Sino Biological) was done by pre-incubation with ALP-conjugated streptavidin (Roche Diagnostic) (1:1 molar ratio) for 20 minutes at RT, before addition to the plates. Bound proteins were detected as above. The set-up was also performed by directly coating the antibody variants (66.87-1.04 nM), followed by addition of biotinylated FcγRIIIb (0.5 μg/ml) pre-incubated with ALP-conjugated streptavidin (1:1 molar ratio).

To study human FcRn binding, titrated amounts of the antibody formats (7.0-0.0032 nM) were coated in wells. 1:1 pre-formed complex of site-specific biotinylated human soluble FcRn (0.25 μg/ml; Immunitrack) and ALP-conjugated streptavidin (Roche Diagnostics) were incubated for 20 minutes at RT before added to the plates. Bound protein was visualized as above. This set-up was performed using PBS/T and PBS/S/T with pH 5.5 or 7.4.

To study human C1q binding, recombinant human Her2 (5.0 or 10.0 μg/ml: Sino Biological) was coated, and titrated amounts of the antibody formats were added (42.0-0.32 nM or 66.87-1.04 nM), respectively. C1q binding was also performed by directly coating of the antibody variants (200.0-3.12 nM). Human C1q (0.336 or 0.5 μg/ml; Complement Technology), diluted in Veronal Buffer (Complement Technology), was added and incubated at 37° C. for 30 minutes, before anti-C1q from rabbit (1:5000 or 1:10000; Dako) was added. Lastly, anti-rabbit-HRP (1:5000; GE Healthcare) was added to the plates, and visualized by addition of 3,3′,5,5′-tetramethybenzidine (TMB) substrate solution (Merck Millipore). Absorbance was measured at 620 nm, or the reaction was stopped by adding 50 μl 1M HCl, and then measured at 450 nm with a Sunrise spectrophotometer (TECAN).

### HERA

HERA was performed as before (Grevys et al., 2018, Nat. Commun., 9 (1): 621) with minor modifications. 7.5×10⁴ HMEC-1 cells, stably expressing HA-hFcRn-EGFP (>100-fold), were seeded into 48-well plates per well (CorningCostar) and cultured for one day in growth medium (Gibco MCDB 131 medium (ThermoFisher), 10% heat-inactivated FCS (Merck), 2 mM L-glutamine (ThermoFisher), 1% Pencillin-Streptomycin (Merck), 10 ng/ml mouse epidermal growth factor (ThermoFisher), 1 μg/ml hydrocortisone (Merck). Stable FcRn expression of the cells was ensured by addition of 5 μg/ml blasticidine (ThermoFisher) and 100 μg/ml G418 (ThermoFisher). The cells were washed and starved for 1 hour in Hank's balanced salt solution (HBSS) (ThermoFisher) before 800 nM of the antibody formats, diluted in 125 μl HBSS (pH 7.4), were added to the cells followed by incubation for 3 hours. Cells were then washed with ice cold HBSS (pH 7.4) before growth medium without FCS, but supplemented with MEM non-essential amino acids (ThermoFisher), were added to the cells, and incubated for additional 3 hours. Medium samples were then collected, and the amounts of antibody present quantified by ELISA. This was done by coating of Her2 as above followed by blocking (PBS/S) and washing (PBS/T) before the HERA samples were added. Binding of the antibodies was detected using an anti-Fc-ALP antibody (Merck) diluted in PBS/S/T (1:5000), and measured as above.

### CDC

A Calcein-AM release CDC assay was performed. 1×10⁷ WSU-NHL, SU-DHL4 cells or Raji cells were stained with 1 μl Calcein AM (Merck) for 30 minutes. Then, the cells were washed with HBSS (Thermofisher) and resuspended in RPMI (Merck) to a cell density of 1×10⁶. Then, 5×10⁴ cells were added to V-shaped 96-well plate together with titrated amounts of anti-CD20 antibodies (140-0.729 nM) and NHS (Complement Technology) (25% final concentration). RIPA buffer (Thermofisher), instead of antibody and NHS, was used to determine maximal lysis of the target cells by 1:1 dilution. Basal release was measured in the absence of antibodies and/or NHS. The plates were incubated at 37° C. for 1 hour and centrifuged at 2000 rpm for 10 minutes. Supernatant was transferred to black clear bottom optical 96-well Viewplates (Perkin Elmer) and fluorescent intensity was measured at 485 nm excitation/510 nm emission using an Envision plate reader (Perkin Elmer) before percent lysis activity of each antibody format was calculated.

### In Vivo Half-Life Studies

The experiments were approved by the Animal Care and Use Committee at The Jackson Laboratory and performed in accordance with the approved guidelines and regulations and carried out at the Jackson Laboratory (JAX Services, Bar Harbor, ME). Briefly, hemizygote Tg32 mice (B6.Cg-Fcgrt^{m1Dcr} Tg (FCGRT)32Dcr/DcrJ; The Jackson Laboratory), expressing human FcRn, but not the mouse counterpart, were used to determine the plasma half-life of the antibody formats. When stated, mice were pre-loaded with 500 mg/kg of intraveneous immunoglobulin (IVIg) (privigen; CSL Behring) 48 hours prior to administration of the Her2 specific antibody formats. Each test article was studied in groups consisting of 5 mice, where only male mice were used (age: 8-9 weeks, weight: 22-27 g). Mice were given equimolar amounts of the test articles (1.0 mg/kg of IgA2 and IgG1, 1.3 mg/kg of the tandem variants) by intravenous injections, and blood samples were taken from the retro-orbital sinus at day 1, 2, 3, 4, 5, 7, 10, 12, 16, 19 and 23 after injection. Immediately after collection, the blood samples were processed, plasma isolated and diluted in 50% glycerol/PBS for storage at −20° C. When quantified in ELISA, plasma samples were diluted 1:50 to 1:400 (or 1:25, 1:50, 1:100 or 1:400) in PBS/S/T. The half-life data are presented as %, calculated by remaining protein at given time points after injection compared to day 1. Prism 8 was used to perform nonlinear regression analysis, and the half-life was calculated using the formula:

$${t}_{1 / 2} = \frac{log 0.5}{( log Ae / A 0 )} \times t ,$$

where t_{1/2} is the half-life of the given antibody, Aₑ is the amount of the antibody remaining, A₀ is the amount of antibody on day 1 and t is the elapsed time.

### Statistical Analysis

Generation of figures and statistical analysis were performed using GraphPad Prism 8 for Mac (Version 8.1.2; GraphPad Software Inc.), Microsoft Excel for Mac (Version 16.35) and BioRender.com.

### Results

### Design and Production of Tandem Antibodies

To design the tandem antibody formats, IgG1 and IgA2 heavy chain (HC) encoding expression vectors, with the Her2 specificity derived from trastuzumab (Carter, P. et al. Proceedings of the National Academy of Sciences of the United States of America 89, 4285-4289 (1992)), were combined with sequences encoding the IgA2-Fc (without the tailpiece) or IgG1-Fc, respectively, where their corresponding hinge sequences were included. In addition to wild-type IgG1, an engineered version with abolished ability to engage the low affinity FcγRs and C1q was included (P329G, L234A and L235A; PGLALA) (Schlothauer, T. et al. Protein engineering, design & selection: PEDS 29, 457-466 (2016)). To produce the antibodies, the constructed vectors were combined with a vector encoding an anti-Her2 human kappa light chain (LC) (Meyer, S. et al. mAbs 8, 87-98 (2016), and Mester, S. et al. mAbs 13, 1893888 (2021)). Schematic illustrations of the antibody variants are shown in FIG. 1a-d.

The purified proteins were analyzed by sodium dodecyl sulphate-polyacrylamide gel electrophoresis (SDS-PAGE), which revealed that the tandem variants migrated with molecular masses about 50 kDa larger than that of the parental antibodies. Under reducing conditions, the variants migrated as two distinct bands corresponding to their HCs (50-75 kDa) and LCs (25 kDa), respectively (data not shown). The tandem variants were produced in similar amounts as that of their parental counterparts (1-2 g/L), and bound recombinant human Her2 in an enzyme-linked immunosorbent assay (ELISA) (data not shown).

The Tandem Designs Bind FcRn and have Extended Plasma Half-Life

To compare the ability of the designed antibodies to bind human FcRn, ELISA was performed (FIG. 2a). Since FcRn binds to the C_{H}2-C_{H}3 elbow region of IgG1 in a strict pH-dependent manner (FIG. 1b), with binding at acidic pH, and no binding or release at neutral pH, we performed the assay at pH 5.5 and pH 7.4. The results demonstrated that both tandem variants bound the receptor in a pH-dependent manner, similar to that of IgG1, while IgA2 did not bind (FIG. 2b).

Next, we used a human endothelial cell-based recycling assay (HERA) (Grevys, A. et al. Nature communications 9, 621 (2018)) to study the ability of the antibodies to be rescued from intracellular degradation by FcRn, using adherent human microvascular endothelial cells (HMEC-1) that stably overexpress human FcRn. Briefly, the antibodies were added to the cells followed by an incubation step, before medium was removed and replaced. After additional incubation, the medium was collected to quantify the amounts of antibody present, as a measurement of rescue from intracellular degradation. The HERA results showed that IgG1, but not IgA2, was rescued from intracellular degradation, as expected. In addition, IgA2-IgG1 was more efficiently rescued than the tandem format with opposite orientation, IgG1-IgA2, and even somewhat more than IgG1 (FIG. 2c).

To address how this translated into in vivo plasma half-lives, we administrated the antibody formats intravenously to human FcRn transgenic mice, followed by collection of blood samples over time before quantification by ELISA (FIG. 2d). While IgA2 was measured with a plasma half-life of only 0.9 day, IgA2-IgG1 showed the greatest increase in half-life (6.8 days), while the half-life of IgG1-IgA2 was only moderate (2.7 days) compared with IgA2. As such, we measured a distinct 2.5-fold difference between the tandem variants, where IgA2-IgG1 showed a half-life closest to that of IgG1 (9.2 days) (FIG. 2e).

### Tandem Variants Display Fc Effector Binding

While the FcαRI binds the CH2-CH3 elbow region of IgA2 (Herr, A. B. et al. Nature 423, 614-620 (2003)), IgG1 engages a range of FcγRs at the lower hinge and upper part of the CH2 domains (Sondermann, P. et al. Nature 406, 267-273 (2000), and Radaev, S et al. The Journal of biological chemistry 276, 16469-16477 (2001)) (FIG. 1b). To investigate the Fc effector molecule binding properties of the tandem variants when captured on the cognate Her2 antigen, we performed a set of ELISAs (FIG. 5a). The results revealed that FcαRI bound both the tandem variants, although slightly weaker than IgA2, while no binding was detected for IgG1 (FIG. 5b). Studying binding to the FcγRs, confirmed that both the tandem variants showed similar binding responses as that of IgG1 (FIG. 5c-i), except for IgA2-IgG1 that bound slightly stronger to FcγRIIb and FcγRIIIa-V, and in particular to the decoy receptor FcγRIIIb. None of the FcγRs, except for FcγRI, bound the PGLALA-containing IgG1 variant, in line with published data (Schlothauer, T. et al. Protein engineering, design & selection: PEDS 29, 457-466 (2016)). IgA2 did not bind any of the FcγRs, as expected.

IgA2 Combined with the Fc from IgG1 or IgG2 Show Fc Receptor Binding

Neutrophils express the FcγRIIIb, which has been shown to act as a decoy receptor that hampers IgG1-mediated ADCC (Brandsma, A. M. et al. Frontiers in immunology 10, 704 (2019), and Treffers, L. W. et al. Frontiers in Immunology 9, 3124 (2018)). However, the IgG2 subclass does not engage this particular receptor (Williams, T. E. et al. Biophys J 79, 1858-1866 (2000), and Bruhns, P. et al. Blood 113, 3716-3725 (2009)). Therefore, we re-designed the preferred tandem orientation by replacing the IgG1-Fc with that of IgG2 (FIG. 3a). The generated anti-Her2 IgG2-based variants were well produced and bound recombinant Her2 (data not shown). Regarding binding to the Fc receptors, IgA2-IgG2 was shown to harbor the capacity to engage FcαRI, but again not to the same extent as IgA2, while both IgG2 and IgA2-IgG2 did show binding to FcγRIIa, but not to FcγRIIIb (FIG. 6). To confirm lack of FcγRIIIb engagement of the IgG2-based variants, high amounts of the antibodies were directly coated in ELISA wells followed by addition of the receptor. The results revealed strict discrimination where IgA2-IgG1 bound equally well as IgG1, while IgG2, IgA2-IgG2 and IgA2 did not bind at all (FIG. 3b).

### IgA2-IgG1 Shows Enhanced C1q Binding

Complement factor C1q binds IgG via the lower hinge and upper part of the CH2 domain, a binding site that partly overlaps with that of the FcγRs (FIG. 1b). However, the capacity of C1q to initiate the complement cascade and induce CDC depends on formation of IgG hexamers via Fc: Fc contacts upon binding to cell-surface antigens (Diebolder, C. A. et al. Science (New York, N.Y.) 343, 1260-1263 (2014)). To measure C1q binding of the designed antibody formats, they were captured on recombinant Her2 coated in ELISA (FIG. 4a). Strikingly, C1q bound more strongly to IgA2-IgG1 than IgG1 followed by the IgG1-IgA2 tandem variant (FIG. 4b-c). No or only weak C1q binding was measured for IgG1-PGLALA (FIG. 4b-d), while IgA2 did not bind (FIG. 4b-c). Thus, C1q binding was enhanced when IgG1-Fc was fused to IgA2, which strongly supports that this tandem design favors Fc: Fc interactions and hexamer formation upon binding to Her2. In addition, as IgG2 only binds weakly to C1q⁶⁹ (FIG. 4d), the tandem IgA2-IgG2 variant did not engage C1q (FIG. 4d).

### Fc-Engineering of IgA2-IgG2 Enhances CDC Activity

To explore the effect of enhanced on-target C1q binding activity of the tandem format in a relevant system, we took advantage of a recently developed chimeric anti-CD20 specific antibody (UMAB2) (Evers, M. et al. mAbs 12, 1795505 (2020), and Meyer, S. et al. British journal of haematology 180, 808-820 (2018)), that can be used in the context of CDC-induced killing of malignant CD20-expressing B cell lines. The variable sequences of this antibody were introduced into vectors encoding IgA2, IgG2 and the IgA2-IgG2 tandem variant. The rationale for this, despite that IgG2 only binds C1q weakly (FIG. 4d), was to introduce three amino acid substitutions (Q311R, M428E and N434W: REW) in the IgG2-based variants to assess the effect. The produced anti-CD20 antibodies migrated with expected molecular masses by SDS-PAGE and showed intact binding integrity in ELISA (FIG. 7).

As soluble recombinant CD20 does not exist, C1q binding was performed by coating the antibody variants directly in ELISA, which showed strongest binding to IgG1 while both IgG2 and IgA2-IgG2 only bound weakly (FIG. 8a). This is in line with data on the anti-Her2 variants when captured on the antigen (FIG. 4d), as well as when coated directly (FIG. 8b). However, when the REW substitutions were introduced, moderate enhanced C1q binding was measured for IgG2 with both specificities (FIG. 8a-b). Similarly, IgA2-IgG2 bound weakly while stronger binding was measured when the REW substitutions were introduced (FIG. 8a-b). Yet, the REW-containing IgA2-IgG2 tandem variants did not bind C1q as strongly as IgG1 (FIG. 8a-b).

In addition, we performed a Calcein AM-based CDC assay that relies on labeling of the target cells with the fluorescent dye Calcein, which is released following antibody-mediated lysis in the presence of NHS (FIG. 8c). Here, we used the high and low CD20-expressing cell lines SU-DHL4 and WSU-NHL, respectively, which confirmed the potent ability of IgA2-IgG2-REW to induce CDC followed by IgA2-IgG2 at high CD20-expression (FIG. 8d-e). IgG2-REW induced CDC beyond that of IgG2, but the activity was lost at lower antibody concentrations, while the CDC activity of the tandem variant was maintained (FIG. 8d). At low CD20-expression, only the REW-containing variants induced CDC-mediated lysis (FIG. 8e).

Taken together, we demonstrate that the REW substitutions enhance the ability of IgG2 to induce CDC, and that the effect is even stronger when the REW-containing IgG2-Fc is combined in tandem with full-length IgA2. This was observed both at high and low levels of CD20-expression.

It was also confirmed that neither IgA2 nor IgA2-IgG2, also in the context of the REW-substitutions, bound the decoy receptor FcγRIIIb (FIG. 9h), while still maintaining the capacity to engage the FcαRI (FIG. 9a). No or only minor differences were measured between the wild-type and REW-containing variants, while the IgG2-based variants bound more selectively to the FcγRs than IgG1 (FIG. 9a-h), in line with expected IgG2 binding properties (Williams, T. E. et al. Biophys J 79, 1858-1866 (2000), and Vidarsson, G. et al. Frontiers in Immunology 5 (2014)).

It was also shown that the REW-containing IgA2-IgG2 bound human FcRn pH-dependently with increased binding at acidic pH (FIG. 10). This strongly suggests that the REW substitutions will translate into extended half-life of the IgA2-IgG2 format.

### Discussion

As shown above we have combined structural features of IgA and IgG to make antibody formats with multiple effector functions. We have identified a format with favorable ability to engage human FcRn. This was achieved with a tandem variant where IgA2 was fused to the IgG1-Fc, yielding efficient rescue from degradation in HERA and 7-fold longer half-life in human FcRn expressing mice compared with IgA2. This gave a 2.5-fold longer half-life than that of the opposite tandem orientation, IgG1-IgA2. Hence, we measured a distinct difference in plasma half-life of the two tandem variants, where adding the IgG1-Fc at the C-terminal end of the IgA2-HCs was shown to be the most optimal orientation for efficient human FcRn-mediated rescue from degradation, both in vitro and in vivo.

Moreover, we studied the capacity of the formats to engage C1q and induce CDC-mediated cell killing. In particular, and advantageously we found that the IgA2-IgG1 orientation gained improved C1q binding compared with IgG1 upon capturing on Her2 in ELISA.

We also explored the IgA2-IgG2 format combined with the REW substitutions. Advantageously, the REW substitutions were shown to improve binding to C1q in ELISA for both IgG2 and IgA2-IgG2, when coated directly (anti-Her2 and anti-CD20).

In line with the effect observed in the C1q binding assay, IgG2-REW gained the ability to mediate CDC, which was most pronounced at higher target concentrations. REW-containing IgA2-IgG2 performed extremely well in the cellular CDC assay when targeting CD20-expressing cancer B cells. This suggests that IgA2-IgG2-REW has a profound ability to form Fc: Fc hexamer structures upon binding to CD20 on the target cells, which allows the complement cascade to be induced far more efficiently than for IgG2-REW. The results also strongly suggest that the REW substitutions will translate into extended half-life of the IgAFc-IgGFc formats. In line with this, the REW-containing IgA2-IgG2 bound human FcRn pH-dependently with increased binding at acidic pH. Thus the REW substitutions have been shown to further enhance the properties of the constructs, e.g. for C1q binding and CDC, and also for FcRn binding.

### Further Results

Potent CDC Activity with the IgG1-Fc at the C-Terminal End

To study CDC activity of the tandem orientations, we performed a Calcein AM release CDC assay that relies on labeling of the target cells with the fluorescent dye Calcein-AM, which is released following antibody-mediated lysis in the presence of NHS (FIG. 8c). Here, we used Raji cells expressing low levels of CD20 relative to the levels of complement inhibitory receptors and is as such considered “hard-to-kill”. These results (FIG. 11a) confirmed the potent ability of the IgA2-IgG1 constructs of the invention to induce CDC and is consistent with efficient C1q binding, similar to that of IgG1-WT, in ELISA, while the opposite tandem orientation IgG1-IgA2 format showed poor binding like that of IgG1-PGLALA (mutant with abolished C1q binding).

### Fc-Engineering Enhances CDC Activity

By introducing the REW mutations into the tandem formats, we showed that the CDC activity could be further enhanced (FIG. 11b). Specifically, the IgA2-IgG1-REW orientation induced higher levels of CDC compared to the opposite tandem orientation. By introducing REW into the IgA2-IgG1 construct we achieved somewhat higher levels of CDC activity than that of IgG1-WT.

Taken together, we demonstrate that the IgA2-IgG1 orientation is superior to the IgG1-IgA2 orientation in engaging C1q and mediating CDC when bound to their target antigen. In other experiments we have also shown that none of the tandem formats could trigger complement activation in human serum in the absence of target binding (data not shown). This is important for the safety aspects of the constructs when used therapeutically.

### Strong Binding of IgA2-IgG1 to FcγRIIIa

NK cells express FcγRIIIa and are thought to be the main cell population to mediate ADCC upon cross-linking of IgG antibodies. Binding to FcγRIIIa of the tandem orientations was therefore studied when captured on the cognate Her2 antigen. The results (see FIG. 12) revealed that both orientations/formats bound the receptor, while the IgA2-IgG1 orientation bound more strongly than the opposite tandem orientation.

Taken together, this indicate that the IgA2-IgG1 orientation may have improved ability to bind to FcγRIIIa on NK cells and as such efficiently mediate ADCC, compared to the opposite orientation.

### The Tandem Formats Bind FcRn

To measure the ability of the tandem formats to bind human FcRn, ELISA was performed (FIG. 2a). Since FcRn binds to the C_{H}2-C_{H}3 elbow region of IgG1 in a strict pH-dependent manner (FIG. 1b), with binding at acidic pH, and no binding or release at neutral pH, we performed the assay at pH 5.5 and pH 7.4. The results (FIG. 13) demonstrated that both tandem variants bound the receptor in a pH-dependent manner, where the IgA2-IgG1 orientation bound stronger than the opposite orientation, and also stronger than IgG1 WT.

When introducing REW into the formats, enhanced binding at pH 5.5 was measured, while low binding was maintained at pH 7.4.

The Tandem Formats have Extended Half-Life in a Competition Model

To measure the in vivo plasma half-lives of the antibody formats in a physiological relevant situation, we administrated the antibodies intravenously to human FcRn transgenic mice that were pre-loaded with IVIg, followed by collection of blood samples over time before quantification by ELISA (FIG. 2d).

The mice have low levels of endogenous mouse IgG as a result of pathogen-free housing, combined with poor ability of mouse IgG to bind human FcRn in the transgenic mice. Therefore, we injected human IgG into the mice to mimic a physiological relevant situation, where high levels of IgG will compete for binding and recycling of FcRn.

While IgA2 had a plasma half-life of only 0.8 days (FIG. 14), IgA2-IgG1 showed increased half-life (3.1 days). However, IgG1 with the same specificity was measured with a slightly longer half-life (4.4 days). When introducing the REW mutations into the tandem format (IgA2-IgG1) a similar half-life to that of IgG1 was measured (4.5 days).

Taken together, the IgA2-IgG1 tandem format is, in contrast to IgA2 alone, handled and rescued by FcRn in the presence of high levels of human IgG that compete for FcRn engagement. The IgA2-IgG1 variant show a reduced half-life compared to that of the parental IgG1, but this is compensated for when introducing REW.

### Example 2: Design of an Alternative (Extended) IgA Fc-IgG Fc Tandem Format

Here, we report on an alternative tandem design that still combines structural elements from an IgA Fc and an IgG Fc, specifically an IgA2 Fc and an IgG1 Fc, allowing for efficient engagement of both the FcγRs and FcαRI, as in Example 1. This format combines an IgA Fc (here IgA2 Fc) and an IgG Fc (here IgG1 Fc) with increased separation (or an extended format) provided by the inclusion of an additional IgG1-CH1 domain. Specifically, an IgG1 Fab and hinge region is fused to the IgA2 Fc, followed by an IgG1 hinge, an IgG1 CH1 domain, a third IgG1 hinge and then a full IgG1 Fc (such a construct is denoted as IgG1 Fab-IgA2 Fc-H1-CH1-H1-IgG1 Fc, see FIG. 15). The design was also combined with the REW Fc-engineering strategy, in which the REW substitutions were introduced into the C-terminal IgG1 Fc domain. Anti-CD20 and Anti-HER2 specificity is provided by the antigen binding fragment in the form of the IgG1 Fab2 fragment.

### Materials and Methods

The antibody production and purification is described in Example 1. The CDC assay was performed as described in Example 1.

### Results

When anti-CD20 IgG1 Fab2-IgA2 Fc-H1-CH1-H1-IgG1 Fc (WT or REW) was investigated for their ability to mediate CDC against “hard-to-kill” target cells in the form of Raji cells which express low levels of CD20, the novel tandem format gave rise to potent killing activity which was even greater than that of the IgA2-IgG1 format as well as IgG1 (FIG. 16). Introduction of the REW substitutions into the C-terminal IgG1 Fc domain resulted in further enhanced CDC activity.

These new extended CH-1 containing formats were also shown to bind equally well to Fcγ receptors as the IgA2-IgG1 containing tandem constructs of the invention described in Example 1, and IgG1-WT, when tested on FcγRIIIa-V (data not shown).

1. A protein construct comprising a human IgA Fc region and a human IgG Fc region, wherein the C-terminals of the human IgA Fc region are connected to the N-terminals of the human IgG Fc region.

2. The protein construct of claim 1, wherein the IgA Fc region is an IgA1 Fc region or IgA2 Fc region, preferably an IgA2 Fc region.

3. The protein construct of claim 1 or claim 2, wherein the IgG Fc region is an IgG1 Fc region or IgG2 Fc region.

4. The protein construct of any one of claims 1 to 3, wherein said construct is capable of binding to Fc receptors or capable of Fc effector function.

5. The protein construct of claim 4, wherein said construct can bind FcαR, bind one or more FcγRs, bind FcRn, bind C1q, induce CDC, induce ADCC and/or induce ADCP.

6. The protein construct of any one of claims 1 to 5, wherein the C-terminals of the human IgA Fc region are connected to the N-terminals of the human IgG Fc region by linkers, and/or by a CH1 domain.

7. The protein construct of claim 6, wherein said linkers are peptide linkers, preferably comprising an antibody hinge region.

8. The protein construct of any one of claims 1 to 7, wherein the IgG Fc region or IgA Fc region comprises mutations or modifications which enhance effector function or increase plasma half-life.

9. The protein construct of claim 8, wherein said mutations or modifications increase binding to Fc receptors, preferably binding to FcRn, or increase C1q binding.

10. The protein construct of claim 8 or claim 9, wherein said Fc region is a modified IgG Fc region characterized by comprising:
(i) an arginine (R) residue, or a similar residue such as a lysine (K) residue, at position 311 (or a position corresponding thereto);
(ii) a glutamic acid (E) residue, or a similar residue such as an aspartic acid (D) residue, at position 428 (or a position corresponding thereto); and
(iii) a tryptophan (W) residue, or a similar residue such as a tyrosine (Y) residue or a phenylalanine (F) residue, at position 434 (or a position corresponding thereto).

11. The protein construct of any one of claims 1 to 10, further comprising a targeting domain, preferably a receptor domain or a receptor ligand, or an antigen binding domain.

12. The protein construct of claim 11, wherein said antigen binding domain is an antibody or an antigen binding fragment thereof.

13. The protein construct of claim 12, wherein said antibody is an IgA antibody or an antigen binding fragment thereof, or an IgG antibody or an antigen binding fragment thereof.

14. The protein construct of any one of claims 11 to 13, wherein said targeting domain binds to a target molecule on a cancer cell or on an infectious pathogen.

15. The protein construct of claim 14, wherein the cancer cell is from a solid tumour or a haematological cancer, or wherein the infectious pathogen is a bacteria.

16. One or more nucleic acid molecules comprising nucleotide sequences that encode the protein construct of any one of claims 1 to 15; or
one or more expression vectors comprising such nucleic acid molecules; or
one or more host cells comprising said expression vectors, nucleic acid molecules, or protein constructs of any one of claims 1 to 15.

17. A method of producing the protein construct of any one of claims 1 to 15, said method comprising the steps of (i) culturing a host cell comprising one or more of the expression vectors or one or more of the nucleic acid sequences as defined in claim 16 under conditions suitable for the expression of the encoded protein construct; and optionally (ii) isolating or obtaining the expressed protein construct from the host cell or from the growth medium/supernatant.

18. The method of claim 17, wherein said method further comprises a step of purification of the protein product and/or formulating the protein product into a composition including at least one additional component.

19. A composition, preferably a pharmaceutically acceptable composition, comprising a protein construct of any one of claims 1 to 15, or one or more nucleic acid molecules or expression vectors of claim 16.

20. The protein construct of any one of claims 1 to 15 for use in therapy.

21. The protein construct for use of claim 20, wherein said protein construct comprises a targeting domain, preferably a receptor domain or a receptor ligand, or an antigen binding domain, that binds to a target or antigen, and wherein said protein construct is for use in the treatment or prevention of a disease that is characterized by the expression of said target or antigen, preferably wherein said disease is cancer or an infectious disease caused by a pathogen expressing said target or antigen.

22. A method of treating or preventing a disease that is characterized by the expression of a target or antigen, preferably wherein said disease is cancer or an infectious disease caused by a pathogen expressing said target or antigen, wherein said method comprises the step of administering to a subject in need thereof a therapeutically effective amount of a protein construct of any one of claims 1 to 15, wherein said protein construct comprises a targeting domain, preferably a receptor domain or a receptor ligand, or an antigen binding domain, that binds to said target or antigen.

23. Use of a protein construct of any one of claims 1 to 15, wherein said protein construct comprises a targeting domain, preferably a receptor domain or a receptor ligand, or an antigen binding domain, that binds to a target or antigen, in the manufacture of a medicament for use in the treatment or prevention of a disease that is characterized by the expression of said target or antigen, preferably wherein said disease is cancer or an infectious disease caused by a pathogen expressing said target or antigen.


SEQUENCE LISTING (44 sequences)

SEQ ID NO: 1 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 3 - protein, 376 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRYTQKSLSLSPG

SEQ ID NO: 4 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCSAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - protein, 333 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 6 - protein, 320 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 7 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 8 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVEHEALHWHYTQKSLSLSPG

SEQ ID NO: 9 - protein, 376 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHRDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVEHEALHWRYTQKSLSLSPG

SEQ ID NO: 10 - protein, 376 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHRDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVEHEALHWHYTQKSLSLSPG

SEQ ID NO: 11 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCSAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVEHEALHWHYTQKSLSLSLG

SEQ ID NO: 12 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 215 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 14 - protein, 216 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRYTQKSLSLSPG

SEQ ID NO: 15 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 210 aa, Homo sapiens
HPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 17 - protein, 210 aa, Homo sapiens
HPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 18 - protein, 15 aa, Homo sapiens
EPKSCDKTHTCPPCP

SEQ ID NO: 19 - protein, 12 aa, Homo sapiens
ERKCCVECPPCP

SEQ ID NO: 20 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 21 - protein, 12 aa, Homo sapiens
ESKYGPPCPPCS

SEQ ID NO: 22 - protein, 22 aa, Homo sapiens
PVPSTPPTPSPSTPPTPSPSCC

SEQ ID NO: 23 - protein, 9 aa, Homo sapiens
RVPPPPPCC

SEQ ID NO: 24 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 25 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 26 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 27 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 28 - protein, 101 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPC

SEQ ID NO: 29 - protein, 101 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPC

SEQ ID NO: 30 - protein, 18 aa, Homo sapiens
PTHVNVSVVMAEVDGTCY

SEQ ID NO: 31 - protein, 565 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 565 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPGK

SEQ ID NO: 33 - protein, 552 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 552 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVEHEALHWHYTQKSLSLSPGK

SEQ ID NO: 35 - protein, 552 aa, synthetic construct
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVEHEALHWHYTQKSLSLSPGK

SEQ ID NO: 36 - protein, 548 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 548 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVEHEALHWHYTQKSLSLSPGK

SEQ ID NO: 38 - protein, 549 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAESKYGPPCPPCSAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 549 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAESKYGPPCPPCSAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVEHEALHWHYTQKSLSLSLGK

SEQ ID NO: 40 - protein, 599 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRYTQKSLSLSPGK

SEQ ID NO: 41 - protein, 599 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHRDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVEHEALHWHYTQKSLSLSPGK

SEQ ID NO: 42 - protein, 668 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 668 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAEPKSCDKTHTCPPCPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPGK

SEQ ID NO: 44 - protein, 5 aa, synthetic construct
GGGGS